(19) United States
(12) Patent Application Publication
(10) Pub. No.: US 2010/0322930 A1
(43) Pub. Date: Dec. 23, 2010

(54) FIBRONECTIN-BASED BINDING MOLECULES AND THEIR USE

(76) Inventors: Frank Kolbinger, Neuenburg (DE); Karen Jane Vincent, Leymen (FR); Barbara Brannetti, Basel (CH); Stefan Ewert, Allschwil (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/810,375

(22) PCT Filed: Dec. 22, 2008
(86) PCT No.: PCT/IB08/03962

§ 371 Date: Sep. 8, 2010

Related U.S. Application Data
(60) Provisional application No. 61/009,361, filed on Dec. 27, 2007.

(51) Int. Cl. A61K 39/395; C07K 14/78; C07K 16/18; C07K 14/765; C07H 21/00; C12N 15/63; C12N 5/10; C12P 21/02; A61K 38/39; G01N 33/53; C07K 14/79; A61P 35/00; A61P 3/00; A61P 25/28; A61P 11/00; A61P 25/18; A61P 9/00; A61P 27/02

(52) U.S. Cl. 424/134.1; 530/395; 530/387.3; 530/363; 536/23.5; 536/23.53; 435/320.1; 435/325; 435/366; 435/358; 435/69.1; 435/69.6; 514/9.3; 436/86; 530/381

(57) ABSTRACT

The invention provides fibronectin-based binding molecules and methods for introducing donor CDRs into a fibronectin-based binding scaffold, in particular, Fn3. The fibronectin-based binding molecules of the invention may be further conjugated to another moiety, for example, Fc, anti-FcRn, HSA, anti-HSA, and PEG, for improved half life and stability, particularly in mammalian cells. The invention also provides methods for screening such molecules for binding to a target antigen as well as the manufacture and purification of a candidate binder.

### RELATED INFORMATION

This application claims the benefit of priority to U.S. Provisional Appln. No. 61/009,361, filed on Dec. 27, 2007. The contents of any patents, patent applications, and references cited throughout this specification are hereby incorporated by reference in their entireties.

### BACKGROUND OF THE INVENTION

Molecules capable of specific binding to a desired target epitope are of enormous importance as both therapeutics and medical diagnostic tools. The exemplar of this class of molecules is the monoclonal antibody. Antibodies can be selected that bind specifically and with high affinity to almost any structural epitope. As a result, antibodies are used routinely as research tools and as FDA approved therapeutics such that the worldwide market for therapeutic and diagnostic monoclonal antibodies is currently worth approximately $30 billion.

However, monoclonal antibodies have a number of shortcomings. For example, classical antibodies are large and complex molecules. They have a heterotetrameric structure comprising two light chains and two heavy chains connected together by both inter and intra disulphide linkages. This structural complexity precludes easy expression of antibodies or multi-specific antibodies such as molecules containing binding specificity for two different molecular therapeutic targets. The large size of antibodies also limits their therapeutic effectiveness since they are often unable to efficiently penetrate certain tissue spaces. In addition, therapeutic antibodies, because they possess an Fc region, occasionally trigger undesired effector cell function and/or clotting cascades.

Accordingly there is a need in the art for alternative binding molecules capable of specific binding to a desired target with high affinity and specificity.

### SUMMARY OF THE INVENTION

The invention solves the foregoing problems by providing fibronectin-based binding molecules and methods for introducing donor CDRs into a fibronectin-based binding scaffold, in particular, Fn3. The fibronectin-based binding molecules of the invention may be further engineered or conjugated to another moiety, for example, PEG, Fc, HSA, anti-HSA for improved half life and stability. The invention also provides methods for screening such molecules for binding to a target antigen as well as the manufacture and purification of a candidate binder. In addition, the present invention demonstrates for the first time that Fn3-based binding molecules are successfully expressed in vivo, particularly in mammalian cells, e.g., rat, mouse, hamster, human cells or cell-lines derived therefrom. Furthermore, the present invention demonstrates that Fn3-based binding molecules engineered or conjugated to another moiety, such as PEG, Fc, HSA, anti-HSA, are also successfully expressed in mammalian cells and show the desired physiological effect of increasing half-life of the molecule.

Accordingly, the invention has several advantages which include, but are not limited to, the following:
providing fibronectin-based binding molecules, for example, modified fibronectin-based binding molecules suitable as therapeutics because of their small size and lack of immunogenicity;
providing fibronectin-based binding molecules having a half-life extension;
providing fibronectin-based binding molecules while also providing a site for linking a desirable functional moiety, such as a blocking moiety, detectable moiety, diagnostic moiety, or therapeutic moiety; and
methods for treating a subject in need of an fibronectin-based binding molecule for diagnosis or therapy.

In one aspect, the invention provides a fibronectin type III (Fn3)-based binding molecule comprising at least two Fn3 beta-strand domain sequences with a loop region sequence linked between each Fn3 beta-strand domain sequence, wherein the loop region sequence comprises a non-Fn3 binding sequence (i.e., an exogenous binding sequence) which binds to a specific target. Typically, the binding molecule further comprises at least one modified amino acid residue compared to the wild-type fibronectin type III (Fn3) molecule (SEQ ID NO: 1) for attaching a functional moiety.

In a particular embodiment, the non-Fn3 binding sequence within the Fn3-based binding molecule comprises all or a portion of a complementarity determining region (CDR), e.g., a CDR of an antibody, particularly a single chain antibody, a single domain antibody or a camelid nanobody. The CDR can be selected from a CDR1, CDR2, CDR3 region, and combinations thereof. Such non-Fn3 binding sequences can be selected to bind to a variety of targets, including but not limited to a cell receptor, a cell receptor ligand, a growth factor, an interleukin, a bacteria, or a virus.

The modified amino acid residue within the Fn3-based binding molecule can include, for example, the addition and/or substitution of at least one Fn3 amino acid residue by at least one cysteine residue or non-natural amino acid residue. In one embodiment, the cysteine or non-natural amino acid residue is located in a loop region, a beta-strand region, a beta-like strand, a C-terminal region, between the C-terminus and the most C-terminal beta strand or beta-like strand, an N-terminal region, and/or between the N-terminus and the most N-terminal beta strand or beta-like strand. In a particular embodiment, the modified amino acid residue includes substitution of one or more of the following residues: Ser 17, Ser 21, Ser 43, Ser 60, Ser 89, Val 11, Leu 19, Thr 58, and Thr 71. In another aspect, the invention provides conjugates which include a fibronectin type III (Fn3)-based binding molecule linked to a non-Fn3 polypeptide, wherein the Fn3-based binding molecule comprises at least two Fn3 beta-strand domain sequences with a loop region sequence linked between each Fn3 beta-strand domain sequence, wherein the loop region binds to a specific target. In another embodiment, the loop region comprises an exogenous binding sequence which binds to a specific target.

Generally, the non-Fn3 polypeptide is capable of binding to a second target and/or increasing the stability (i.e., half-life) of the Fn-3 based binding molecule when administered in vivo. Suitable non-Fn3 polypeptides include, but are not limited to, antibody Fc regions, Human Serum Albumin (HSA) (or portions thereof) and/or polypeptides which bind to HSA or other serum proteins with increased half-life, such as, e.g., transferrin.

The non-Fn3 moiety increases the half-life of the conjugate such that it is greater than that of the unconjugated Fn3-based binding molecule. The half life of the conjugate is at least 2-5 hours, 5-10 hours, 10-15 hours, 15-20 hours, 20-25 hours, 25-30 hours, 35-40 hours, 45-50 hours, 50-55 hours, 55-60 hours, 60-65 hours, 65-70 hours, 75-80 hours, 80-85 hours, 85-90 hours, 90-95 hours, 95-100 hours, 100-150 hours, 150-200 hours, 200-250 hours, 250-300 hours, 350-400 hours, 400-450 hours, 450-500 hours, 500-550 hours, 550-600 hours, 600-650 hours, 650-700 hours, 700-750 hours, 750-800 hours, 800-850 hours, 850-900 hours, 900-950 hours, 950-1000 hours, 1000-1050 hours, 1050-1100 hours, 1100-1150 hours, 1150-1200 hours, 1200-1250 hours, 1250-1300 hours, 1300-1350 hours, 1350-1400 hours, 1400-1450 hours, 1450-1500 hours greater than that of the unconjugated Fn3-based binding molecule. The half life of the conjugate is at least 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold greater than that of the unconjugated Fn3-based binding molecule.

In one embodiment, the non-Fn3 moiety is an antibody Fc region fused to the Fn3-based binding molecule. The half life of this conjugate is at least 5-30 fold greater than that of the unconjugated Fn3-based binding molecule and the in vivo half life of the conjugate is at least 9.4 hours. In another embodiment, the non-Fn3 moiety is serum albumin or transferrin, or a portion thereof, linked to the Fn3-based binding molecule. The half life of this conjugate is at least 25-50 fold greater than that of the unconjugated Fn3-based binding molecule and the in vivo half life of the conjugate is at least 19.6 hours. In another embodiment, the non-Fn3 moiety is an anti-serum albumin or anti-transferrin, or a portion thereof, linked to the Fn3-based binding molecule. The half life of this conjugate is at least 10-35 fold greater than that of the unconjugated Fn3-based binding molecule and the in vivo half life of the conjugate is at least 7.7 hours. In another embodiment, the non-Fn3 moiety is polyethylene glycol, (PEG) linked to the Fn3-based binding molecule. The half life of this conjugate is at least 5-25 fold greater than that of the unconjugated Fn3-based binding molecule and the in vivo half life of the conjugate is at least 3.6 hours.

In one embodiment, the non-Fn3 moiety comprises an antibody Fc region which is fused to the Fn3-based binding molecule at the N-terminal region or the C-terminal region. The antibody Fc region may also be fused to the Fn3-based binding molecule at a region selected from the group consisting of a loop region, a beta-strand region, a beta-like strand, a C-terminal region, between the C-terminus and the most C-terminal beta strand or beta-like strand, an N-terminal region, and between the N-terminus and the most N-terminal beta strand or beta-like strand. The half-life of the Fc conjugate is increased in vivo by at least about 9.4 hours.

In another embodiment, the non-Fn3 moiety comprises a Serum Albumin (SA) such as human serum albumin (HSA), or portion thereof, or a polypeptide which binds SA, such as anti-HSA. The half-life of the SA conjugate in vivo is at least about 19.6 hours, while the half-life of the anti-SA conjugate in vivo is at least about 7.7 hours

In yet another embodiment, the non-Fn3 moiety comprises polyethylene glycol (PEG). The PEG moiety is attached to a thiol group or an amine group. The PEG moiety is attached to the Fn3-based binding molecule by site directed pegylation, for example to a Cys residue, or to a non-natural amino acid residue. The PEG moiety is attached on a region in the Fn3-based binding molecule selected from the group consisting of a loop region, a beta-strand region, a beta-like strand, a C-terminal region, between the C-terminus and the most C-terminal beta strand or beta-like strand, an N-terminal region, and between the N-terminus and the most N-terminal beta strand or beta-like strand. The PEG moiety has a molecular weight of between about 2 kDa and about 100 kDa. The half life of the PEG conjugate is increased in vivo by at least about 3.6 hours.

In another embodiment, the invention pertains to a conjugate with improved pharmacokinetic properties, the conjugate comprising: a fibronectin type III (Fn3)-based binding molecule linked to a polypeptide that binds to an antibody Fc region, wherein the Fn3-based binding molecule comprises at least two Fn3 beta-strand domain sequences with a loop region sequence linked between each Fn3 beta-strand domain sequence, and wherein the conjugate binds to a specific target and has a serum half-life of at least 9.4 hours.

In another embodiment, the invention pertains to a conjugate with improved pharmacokinetic properties, the conjugate comprising: a fibronectin type III (Fn3)-based binding molecule linked to a Serum Albumin (SA) moiety, wherein the Fn3-based binding molecule comprises at least two Fn3 beta-strand domain sequences with a loop region sequence linked between each Fn3 beta-strand domain sequence, and wherein the conjugate binds to a specific target and has a serum half-life of at least 19.6 hours.

In another embodiment, the invention pertains to a conjugate with improved pharmacokinetic properties, the conjugate comprising: a fibronectin type III (Fn3)-based binding molecule linked to a polypeptide that binds to a Serum Albumin (SA) moiety, wherein the Fn3-based binding molecule comprises at least two Fn3 beta-strand domain sequences with a loop region sequence linked between each Fn3 beta-strand domain sequence, and wherein the conjugate binds to a specific target and has a serum half-life of at least 7.7 hours.

In another embodiment, the invention pertains to conjugate with improved pharmacokinetic properties, the conjugate comprising: a fibronectin type III (Fn3)-based binding molecule linked to a PEG moiety, wherein the Fn3-based binding molecule comprises at least two Fn3 beta-strand domain sequences with a loop region sequence linked between each Fn3 beta-strand domain sequence, and wherein the conjugate binds to a specific target and has a serum half-life of at least 3.6 hours.

In another embodiment, the invention pertains to conjugate with improved pharmacokinetic properties, the conjugate comprising: a fibronectin type III (Fn3)-based binding molecule linked to an anti-FcRn moiety, wherein the Fn3-based binding molecule comprises at least two Fn3 beta-strand domain sequences with a loop region sequence linked between each Fn3 beta-strand domain sequence, and wherein the conjugate binds to neonatal FcR receptor (FcRn) with a high affinity at an acidic pH and with a low affinity at a neutral pH. The acid pH can range from about 1 to about 7, and the neutral pH is about 7.0 to about 8.0. In one embodiment, the acidic pH is about pH 6.0 and the neutral pH is about pH 7.4.

The Fn-3 based binding molecules or conjugates can have the Fn3 domain derived from at least two same or different fibronectin modules from any one of the 1Fn-17Fn modules and can be combined in any combination e.g., ¹⁰Fn3-¹⁰Fn3; ¹⁰Fn3-⁹Fn3, ¹⁰Fn3-⁸Fn3, ⁹Fn3-⁸Fn3. Conjugates such as ¹⁰Fn3-¹⁰Fn3-HSA, or anti-HSA or Fc, or PEG; ¹⁰Fn3-⁹Fn3-HSA, or anti-HSA or Fc, or PEG, ¹⁰Fn3-⁸Fn3-HSA, or anti-HSA or Fc, or PEG, ⁹Fn3-⁸Fn3-HSA, or anti-HSA or Fc, or PEG, are also considered to be within the scope of the invention.

The Fn-3 based binding molecules or conjugates can have Fn3 domain derived from at least three or more of the same or different fibronectin modules. e.g., ¹⁰Fn3-¹⁰Fn3-¹⁰Fn3 (-¹⁰Fn3)n, wherein n is any number of 2-10 ¹⁰Fn3 domains; ¹⁰Fn3-⁹Fn3-⁸Fn3 (-Fn3)n, wherein n is any number of 2-10 Fn3 domains; ⁹Fn3-⁸Fn3-⁷Fn3(-Fn3)n, wherein n is any number of 2-10 Fn3 domains. Conjugates of these molecules are also within the scope of the invention.

The invention further pertains to nucleic acids comprising a sequence encoding a Fn-3 based binding molecule or conjugate, expression vector comprising the nucleic acids operably linked with a promoter, cells comprising the nucleic acids and methods of producing a Fn-3 based binding molecule or conjugate that binds to a target by expressing the nucleic acid comprising a sequence encoding the Fn-3 based binding molecule or conjugate in a cell, particularly in a cell in vivo. In a particular embodiment, the cells are mammalian cells, e.g., rat, mouse, hamster, human cells or cell-lines derived therefrom.

Fn3-based binding molecules of the invention can be based on the (e.g., human) wild-type Fn3 sequence, as well as modified version of this sequence, as discussed herein. For example, the Fn3-based binding molecule can be a chimera having Fn3 beta-strands that are derived from at least two different fibronectin modules. Examples of possible chimeras are shown in FIG. 6.

Also provided by the invention are compositions comprising the Fn-3 based binding molecules and conjugates of the invention, formulated with a suitable carrier.

The Fn-3 based binding molecules and conjugates of the invention can be used in a variety of therapeutic and diagnostic applications including, but not limited to, any application that antibodies can be used in. Such uses include, for example, treatment and diagnosis of a disease or disorder that includes, but is not limited to, an autoimmune disease, an inflammation, a cancer, an infectious disease, a cardiovascular disease, a gastrointestinal disease, a respiratory disease, a metabolic disease, a musculoskeletal disease, a neurodegenerative disease, a psychiatric disease, an opthalmic disease and transplant rejection

Other features and advantages of the invention will be apparent from the following detailed description and claims.

### DETAILED DESCRIPTION OF THE INVENTION

In order to provide a clear understanding of the specification and claims, the following definitions are conveniently provided below.

### DEFINITIONS

As used herein, the term “Fibronectin type III domain” or “Fn3 domain” refers to a wild-type Fn3 domain from any organism, as well as chimeric Fn3 domains constructed from beta strands from two or more different Fn3 domains. As is known in the art, naturally occurring Fn3 domains have a beta-sandwich structure composed of seven beta-strands, referred to as A, B, C, D, E, F, and G, linked by six loops, referred to as AB, BC, CD, DE, EF, and FG loops (See e.g., Bork and Doolittle, Proc. Natl. Acad. Sci. U.S.A 89:8990, 1992; Bork et al., Nature Biotech. 15:553, 1997; Meinke et al., J. Bacteriol. 175:1910, 1993; Watanabe et al., J. Biol. Chem. 265:15659, 1990; Main et al., 1992; Leahy et al., 1992; Dickinson et al., 1994; U.S. Pat. No. 6,673,901; Patent Cooperation Treaty publication WO/03104418; and, US patent application 2007/0082365, the entire teachings of which are incorporated herein by reference). Three loops are at the top of the domain (the BC, DE and FG loops) and three loops are at the bottom of the domain (the AB, CD and EF loops) (see FIG. 1). In a particular embodiment, of the invention, the Fn3 domain is from the tenth Fn3 domain of human Fibronectin (¹⁰Fn3) (SEQ. ID. NO: 1).

As used herein the term “Fn3-based binding molecule” or “fibronectin type III (Fn3)-based binding molecule” refers to an Fn3 domain that has been altered to contain one or more non-Fn3 binding sequences.

The term “non-Fn3 binding sequence” refers to an amino acid sequence which is not present in the naturally occurring (e.g., wild-type) Fn3 domain, and which binds to a specific target. Such non-Fn3 binding sequences are typically introduced by modifying (e.g., by substitution and/or addition) the wild-type Fn3 domain. This can be achieved by, for example, random or predetermined mutation of amino acid residues within the wild-type Fn3 domain. Additionally or alternatively, the non-Fn3 binding sequence can be partly or entirely exogenous, that is, derived from a different genetic or amino acid source. For example, the exogenous sequence can be derived from a hypervariable region of an antibody, such as one or more CDR regions having a known binding specificity for a known target antigen. Such CDRs can be derived from a single antibody chain (e.g. a variable region of a light or heavy chain) or a from combination of different antibody chains. The CDRs can also be derived form two different antibodies (e.g., having different specificities). In a particular embodiment, the CDR(s) are derived from a nanobody, for example, a Camelidae-like heavy chain.

The term “complementarity determining region (CDR)” refers to a hypervariable loop from an antibody variable domain or from a T-cell receptor. The position of CDRs within a antibody variable region have been precisely defined (see, Kabat, E. A., et al. Sequences of Proteins of Immunological Interest, Fifth Edition, U.S. Department of Health and Human Services, NIH Publication No. 91-3242, 1991, and Chothia, C. et al., J. Mol. Biol. 196:901-917, 1987, which are incorporated herein by reference).

The term “single domain antibodies” refers to any naturally-occurring single variable domain antibody or corresponding engineered binding fragment, including human domain antibodies as described by e.g. Domantis (Domantis/GSK (Cambridge, UK) (see, e.g., Ward et al., 1989, Nature 341(6242):484-5; WO04058820), or camelid nanobodies as defined hereafter.

The term “single chain antibody” refers to an antibody composed of an antigen binding portion of a light chain variable region and an antigen binding portion of a heavy chain variable region, joined, e.g., using recombinant methods, by a synthetic linker that enables the chains to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. U.S.A 85:5879-5883).

The term “camelid nanobody” refers to a region of camelid antibody which is the small single variable domain devoid of light chain and that can be obtained by genetic engineering to yield a small protein having high affinity for a target, resulting in a low molecular weight antibody-derived protein. See, e.g., WO07042289 and U.S. Pat. No. 5,759,808 issued Jun. 2, 1998; see also, e.g., Stijlemans, B. et al., 2004, J Biol. Chem. 279(2):1256-61. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. This further reduces the already the naturally low antigenicity of camelid antibodies when administered to humans.

The term “target” refers to an antigen or epitope recognized (i.e., bound by) Fn3-based binding molecule of the invention. Targets include, but are not limited to, epitopes present on proteins, peptides, carbohydrates, and/or lipids.

The term “conjugate” refers to an Fn3-based binding molecule chemically or genetically linked to one or more non-Fn3 moieties.

The term “non-Fn3 moiety” refers to a biological or chemical entity that imparts additional functionality to a molecule to which it is attached. In a particular embodiment, the non-Fn3 moiety is a polypeptide, e.g., a serum albumin such as human serum albumin (HSA) or a fragment or mutant thereof, an anti-HSA, or a fragment or mutant thereof, an antibody Fc, or a fragment or mutant thereof, or a chemical entity, e.g., polyethylene gycol (PEG) which increases the half-life of the Fn3-based binding molecule in vivo.

The term “non-natural amino acid residue” refers to an amino acid residue that is not present in the naturally occurring (wild-type) Fn3 domain and includes, e.g., chemically modified amino acids. Such non-natural amino acid residues can be introduced by substitution of naturally occurring amino acids, and/or by insertion of non-natural amino acids into the naturally occurring amino acid Fn3 sequence (see e.g. Sakamoto et al., 2002, Nucleic Acids Research, 30(21) 4692-4699). The non-natural amino acid residue also can be incorporated such that a desired functionality is imparted to the Fn3-based binding molecule, for example, the ability to link a functional moiety (e.g., PEG).

The term “polyethylene glycol” or “PEG” refers to a polyalkylene glycol compound or a derivative thereof, with or without coupling agents or derviatization with coupling or activating moieties.

The term “specific binding” or “specifically binds to” refers to the ability of an Fn3-based binding molecule to bind to a target with an affinity of at least 1×10⁻⁶ M, and/or bind to a target with an affinity that is at least two-fold, (preferably at least 10 fold), greater than its affinity for a nonspecific antigen at room temperature under standard physiological salt and pH conditions, as measured by surface plasmon resonance.

### BRIEF DESCRIPTION OF THE DRAWINGS

FIG. 1A shows the tenth type III module of the wildtype fibronectin molecule with a stick representation of the serine residues, and FIG. 1B shows the amino acid sequence of Fn3 in its secondary structure context. Residues in a beta strand are shown as white circles. Those residues whose side chain forms the hydrophobic core are enclosed with a thicker line. Loop residues are shown shaded. The arrows mark the position in the loops where Fn3 was separated to generate complementary fragments

FIG. 2 shows the tenth type III module of the wildtype fibronectin molecule with proposed serine residues available for modifications (Ser 17-Ser 21-Ser 43-Ser 60-Ser 89).

FIG. 3 shows the three-stranded sheet (strands A-B-E) of the tenth type III module of the wildtype fibronectin molecule. At the bottom of the sheet the candidate residues, Ser 17 and Ser 60, are located. The candidate residue, Ser 21, is located at the top. Ser 55 has been excluded because it is close to the binding surface. Other potential candidate residues are shown, i.e., Val 11, Leu 19, and Thr 58.

FIG. 4 shows the four-stranded sheet of the tenth type III module of the wildtype fibronectin molecule (the other side of the scaffold). Thr 71 is located close to Ser 89 and is also a potential candidate for modification.

FIG. 5 shows the FG and CD loops of the tenth type III module of the wildtype fibronectin molecule.

FIG. 6 A-B shows various combinations the beta-strands of modules 7, 8, 9, and 10 type III module of the wildtype fibronectin molecule to produce fibronectin-based binding molecule chimeras (beta-strand swapping).

FIG. 7 A-C provides information regarding exemplary targets.

FIG. 8 shows the results of the SDS PAGE analysis of Wild type 10Fn3 (RGD to RGA) and wild type 10Fn3 (RGD to RGA)_cys, without a reducing agent (FIG. 8A) and wild type 10Fn3 (RGD to RGA)_{—}30 kDa PEG with a reducing agent (FIG. 8B).

FIG. 9 shows the (Pharmacokinetics) PK in Lewis rat for wild type 10Fn3 (RGD to RGA) using an E. coli expression system.

FIG. 10 shows the PK in Lewis rat for wild type 10Fn3 (RGD to RGA)-PEG using an E. coli expression system.

FIG. 11 shows that calculated half life for wild type 10Fn3 (RGD to RGA) and wild type 10Fn3 (RGD to RGA)-PEG as analyzed by WinNonLin software.

FIG. 12 shows the results of SDS PAGE analysis of wild type 10Fn3 (RGD to RGA)-RSA with reducing agent (FIG. 12a) and wild type 10Fn3 (RGD to RGA)-HSA with reducing agent (FIG. 12b).

FIG. 13 shows the PK in Lewis rat for wild type 10Fn3 (RGD to RGA)-RSA; using a mammalian expression system.

FIG. 14 shows the PK in Lewis rat for wild type 10Fn3 (RGD to RGA)-HSA; using a mammalian expression system.

FIG. 15 shows the calculated half life for wild type 10Fn3 (RGD to RGA) and wild type 10Fn3 (RGD to RGA)-RSA and HSA, as analyzed by WinNonLin software.

FIG. 16 shows the results of the SDS PAGE analysis of VEGFR 10Fn3 binder-RSA with reducing agent (FIG. 16a) and VEGFR 10Fn3 binder-HSA with reducing agent (FIG. 16b).

FIG. 17 is a graph showing the results of an ELISA with VEGFR 10Fn3 binder-HSA and RSA.

FIG. 18 shows the PK in Lewis rat for VEGFR-binding Fn3-HSA using a mammalian expression system.

FIG. 19 shows the PK in Lewis rat for VEGFR-binding Fn3-RSA using a mammalian expression system.

FIG. 20 shows the calculated half life for VEGFR-binding Fn3-HSA and VEGFR-binding Fn3-RSA, as analyzed by WinNonLin software

FIG. 21 shows the results of SDS PAGE analysis of wild type 10Fn3 (RGD to RGA)-anti RSA with reducing agent.

FIG. 22 shows the PK in Lewis rat for wild type 10Fn3 (RGD to RGA)-antiRSA using an E. coli expression system.

FIG. 23 shows the calculated half life for wild type 10Fn3 (RGD to RGA) and wild type 10Fn3 (RGD to RGA)-anti-RSA, as analyzed by WinNonLin software.

FIG. 24 shows the SDS PAGE analysis of wild type 10Fn3 (RGD to RGA) Fc with reducing agent.

FIG. 25 shows the PK in Lewis rat for wild type 10Fn3 (RGD to RGA)-Fc; using a mammalian expression system.

FIG. 26 shows the calculated half life for wild type 10Fn3 (RGD to RGA) and wild type 10Fn3 (RGD to RGA)-Fc, as analyzed by WinNonLin software.

### OVERVIEW

The invention provides fibronectin-based binding molecules and methods for introducing donor CDRs into a fibronectin-based binding scaffold, in particular, Fn3. The invention, as further discussed below, also provides methods for introducing into a fibronectin-based binding molecule, or scaffold, an amino acid residue that is suitable for being conjugated to a moiety. This advantage allows for the fibronectin-based binding molecules of the invention to be further conjugated to other such molecules to build bi- and multi-specific binding molecules and/or allow for the linkage to a moiety such as PEG, for improved half-life and stability.

The invention also provides methods for screening such binding molecules for specific binding to a target, typically a protein antigen, as well as the manufacture of the molecules in, for example, prokaryotic or eukaryotic systems.

In addition, the invention provides methods for the purification of a candidate binding molecule and its formulation.

Still further, the invention provides methods for using such formulated binding molecules in a variety of diagnostic and therapeutic applications, in particular, for the diagnosis or treatment of human disease.

### Fibronectin-Based Binding Scaffolds and Modifications Thereof

In one aspect the invention provides improved scaffolds for making binding molecules. Scaffolds suitable for use in the invention include, but are not limited to, ankyrin repeat scaffolds or one or more members of the immunoglobulin superfamily, for example, antibodies or fibronectin domains.

In one embodiment, the Fibronectin type III domain (Fn3) serves as a scaffold molecule (U.S. Pat. No. 6,673,901, Patent Cooperation Treaty publication WO/03104418, and U.S. patent application 20070082365). This domain occurs more than 400 times in the protein sequence database and has been estimated to occur in 2% of the proteins sequenced to date, including fibronectins, tenascin, intracellular cytoskeletal proteins, and prokaryotic enzymes (Bork and Doolittle, Proc. Natl. Acad. Sci. U.S.A 89:8990, 1992; Bork et al., Nature Biotech. 15:553, 1997; Meinke et al., J. Bacteriol. 175:1910, 1993; Watanabe et al., J. Biol. Chem. 265:15659, 1990). The 3D structure of Fn3 has been determined by NMR (Main et al., 1992) and by X-ray crystallography (Leahy et al., 1992; Dickinson et al., 1994). The structure is described as a beta-sandwich similar to that of an antibody VH domain except that Fn3 has seven β-strands instead of nine. There are three loops on each end of each Fn3 domain; the positions of the BC, DE and FG loops approximately correspond to those of CDR1, 2 and 3 of the VH domain of an antibody, respectively (U.S. Pat. No. 6,673,901, Patent Cooperation Treaty publication WO/03104418). Any Fn3 domain from any species is suitable for use in the invention.

In another embodiment, the Fn3 scaffold is the tenth module of human Fn3 (¹⁰Fn3), which comprises 94 amino acid residues. The three loops of ¹⁰Fn3 corresponding to the antigen-binding loops of the IgG heavy chain run between amino acid residues 21-31 (BC), 51-56 (DE), and 76-88 (FG) (U.S. patent application number 20070082365). These BC, DE and FG loops can be directly substituted by CDR1, 2, and 3 loops from an antibody variable region, respectively, in particular from CDRs of a single domain antibody.

Although ¹⁰Fn3 represents one embodiment of the Fn3 scaffold for the generation of Fn3-based binding molecules, other molecules may be substituted for ¹⁰Fn3 in the molecules described herein. These include, without limitation, human fibronectin modules ¹Fn3-⁹Fn3 and ¹¹Fn3-¹⁷Fn3 as well as related Fn3 modules from non-human animals and prokaryotes. In addition, Fn3 modules from other proteins with sequence homology to ¹⁰Fn3, such as tenascins and undulins, may also be used. Modules from different organisms and parent proteins may be most appropriate for different applications; for example, in designing an antibody mimic, it may be most desirable to generate that protein from a fibronectin or fibronectin-like molecule native to the organism for which a therapeutic or diagnostic molecule is intended.

In another embodiment, the Fn3 is from a species other than human. Non-human Fn3 may cause a detrimental immune response if administered to human patients. To prevent this, the non-human Fn3 can be genetically engineered to remove antigenic amino acids or epitopes. Methods for identifying the antigenic regions of the non-human Fn3 include, but are not limited to, the methods described in U.S. Pat. No. 6,673,580.

In another embodiment, the Fn3 scaffold is a chimera constructed from portions of one or more Fn3, e.g., at least two different Fn3, such as ¹⁰Fn3 and ⁹Fn3. Using the known amino acid sequences and 3D structure of Fn3 domains, the skilled worker can easily identify the regions of different Fn3 molecules that could be combined to make a functional chimeric Fn3 molecule. Such chimeric Fn3 domains can be constructed in several ways including, but not limited to, PCR-based or enzyme-mediate genetic engineering, ab initio DNA or RNA synthesis or cassette mutagenesis.

The above mentioned fibronectin-based binding scaffolds can be constructed ab intio or informed by the use of in silico molecular modeling. In silico or computer aided modeling can include simple nucleic acid or amino acid sequence alignment or 3-D modeling using, for example, Ras-Mol. The modeling of the scaffolds allows for a rational approach as to which regions or loops of the scaffold can be selected for presenting a hypervariable region. Modeling also allows for how to best modify the scaffolds for optimal presentation of one or more hypervariable regions.

Methods for Grafting Hypervariable Regions/CDRs onto a Fibronectin-Based Binding Scaffold

In one aspect, the present invention features improved methods for grafting Hypervariable Regions from other Ig superfamily molecules into the fibronectin-based binding scaffolds of the invention.

In one embodiment, one or more CDRs from an antibody variable region, for example, a heavy chain variable region, light chain variable region, or both, are grafted into one or more loops of one of the above mentioned binding scaffolds. The CDR regions of any antibody variable region, or antigen binding fragments thereof, are suitable for grafting. The CDRs can be obtained from the antibody repertoire of any animal including, but not limited to, rodents, primates, camelids or sharks. In a particular embodiment, the CDRs are obtained from CDR1, CDR2 and CDR3 of a single domain antibody, for example a nanobody. In a more specific embodiment, CDR1, 2 and 3 of a single domain antibody, such as a nanobody, are grafted into BC, DE and FG loops of an Fn3 domain, thereby providing target binding specificity of the original nanobody to the Fibronectin-based binding molecule. Engineered libraries of camelid antibodies and antibody fragments are commercially available, for example, from Ablynx, Ghent, Belgium. The antibody repertoire can be from animals challenged with one or more antigens or from naïve animals that have not been challenged with antigen. Additionally or alternatively, CDRs can be obtained from antibodies, or antigen binding fragments thereof, produced by in vitro or in vivo library screening methods, including, but not limited to, in vitro polysome or ribosome display, phage display or yeast display techniques. This includes antibodies not originally generated by in vitro or in vivo library screening methods but which have subsequently undergone mutagenesis or one or more affinity maturation steps using in vitro or in vivo screening methods. Example of such in vitro or in vivo library screening methods or affinity maturation methods are described, for example, in U.S. Pat. Nos. 7,195,880; 6,951,725; 7,078,197; 7,022,479; 5,922,545; 5,830,721; 5,605,793, 5,830,650; 6,194,550; 6,699,658; 7,063,943; 5,866,344 and Patent Cooperation Treaty publications WO06023144.

Methods to identify antibody CDRs are well known in the art (see Kabat et al., U.S. Dept. of Health and Human Services, “Sequences of Proteins of Immunological Interest” (1983); Chothia et al., J. Mol. Biol. 196:901-917 (1987); MacCallum et al., J. Mol. Biol. 262:732-745 (1996)). The nucleic acid encoding a particular antibody can be isolated and sequenced, and the CDR sequences deduced by inspection of the encoded protein with regard to the established antibody sequence nomenclature. Methods for grafting hypervariable regions or CDRs into a fibronectin-based binding scaffold of the invention include, for example, genetic engineering, de novo nucleic acid synthesis or PCR-based gene assembly (see for example U.S. Pat. No. 5,225,539).

### Methods for Identifying Fibronectin-Based Binding Scaffold Residues Suitable for Modification for Improved CDR Presentation/Binding

The above techniques allow for the identification of a suitable scaffold loop for selection and presentation of a hypervariable region or CDR. However, additional metrics can be invoked to further improve the fit and presentation of the hypervariable region based on structural modeling of the Fn3 domain and the donor antibody.

In one aspect, specific amino acid residues in any of the beta-strands of an Fn3 scaffold are mutated to allow the CDR loops to adopt a conformation that retains or improves binding to antigen. This procedure can be performed in an analogous way to that CDR grafting into a heterologous antibody framework, using a combination of structural modeling and sequence comparison. In one embodiment, the Fn3 residues adjacent to a CDR are mutated in a similar manner to that performed by Queen et al. (see U.S. Pat. Nos. 6,180,370; 5,693,762; 5,693,761; 5,585,089; 7,022,500). In another embodiment, Fn3 residues within one Van der Waals radius of CDR residues are mutated in a similar manner to that performed by Winter et al. (see U.S. Pat. Nos. 6,548,640; 6,982,321). In another embodiment, Fn3 residues that are non-adjacent to CDR residues but are predicted, based upon structural modeling of the Fn3 domain and the donor antibody, to modify the conformation of CDR residues are mutated in a similar manner to that performed by Carter et al. or Adair et al (see U.S. Pat. Nos. 6,407,213; 6,639,055; 5,859,205; 6,632,927)

In another aspect, an Fn3 scaffold containing one or more grafted antibody CDRs is subject to one or more in vitro or in vivo affinity maturation steps. Any affinity maturation approach can be employed that results in amino acid changes in the Fn3 scaffold or the CDRs that improve the binding of the Fn3/CDR to the desired antigen. These amino acid changes can, for example, be achieved via random mutagenesis, “walk though mutagenesis, and “look through mutagenesis. Such mutagenesis of a monobody can be achieved by using, for example, error-prone PCR, “mutator” strains of yeast or bacteria, incorporation of random or defined nucleic acid changes during ab inito synthesis of all or part of a monobody. Methods for performing affinity maturation and/or mutagenesis are described, for example, in U.S. Pat. Nos. 7,195,880; 6,951,725; 7,078,197; 7,022,479; 5,922,545; 5,830,721; 5,605,793, 5,830,650; 6,194,550; 6,699,658; 7,063,943; 5,866,344 and Patent Cooperation Treaty publications WO06023144. New CDR sequences comprising minimal essential binding determinants can also be screened using Kalobios technology as described in US20050255552.

### Engineered and Modified Fibronectin-Based Binding Molecules

In another aspect, the present invention features fibronectin-based binding molecules which have been modified to have altered properties compared to the original fibronectin-based molecule. Modifications include conjugating or fusing the molecule to another molecule, as well as chemically modifying the molecule or altering the amino acid residues or nucleotides of the molecule structure.

### Fibronectin Fusions

The fibronectin-based binding molecules of the present invention can be fused or conjugated to another molecule. Such conjugates are referred to herein as “Fn fusions.” For example, Fn fusions include a fibronectin-based binding molecule fused to a molecule which increases the stability or half-life of the binding molecule (e.g., an Fc region, HSA, or an anti-HSA binding molecule).

For example, Fn fusions may be integrated with the human immune response by fusing the constant region of an IgG (Fc) with a ¹⁰Fn3 module, preferably through the C-terminus of ¹⁰Fn3. The Fc in such a ¹⁰Fn3-Fc fusion molecule activates the complement component of the immune response and increases the therapeutic value of the antibody mimic. Similarly, a fusion between ¹⁰Fn3 and a complement protein, such as C1q, may be used to target cells, and a fusion between ¹⁰Fn3 and a toxin may be used to specifically destroy cells that carry a particular antigen. In addition, ¹⁰Fn3 in any form may be fused with albumin to increase its half-life in the bloodstream and its tissue penetration. Any of these fusions may be generated by standard techniques, for example, by expression of the fusion protein from a recombinant fusion gene constructed using publically available gene sequences.

The Fn fusion may also be generated using the neonatal Fc receptor (FcRn), also termed “Brambell receptor”, which is involved in prolonging the life-span of albumin in circulation (see Chaudhury et al., (2003) J. Exp. Med., 3: 315-322; Vaccarao et al., (2005) Nature Biotech. 23: 1283-1288). The FcRn receptor is an integral membrane glycoprotein consisting of a soluble light chain consisting of β-2-microglobulin, noncovalently bound to a 43 kD α chain with three extracellular domains, a transmembrane region and a cytoplasmic tail of about 50 amino acids. The cytoplasmic tail contains a dinucleotide motif-based endocytosis signal implicated in the internalization of the receptor. The α chain is a member of the nonclassical MHC I family of proteins. The β 2m association with the cc chain is critical for correct folding of FcRn and exiting the endoplasmic reticulum for routing to endosomes and the cell surface.

The overall structure of FcRn is similar to that of class I molecules. The α-1 and α-2 regions resemble a platform composed of eight antiparallel β strands forming a single β-sheet topped by two antiparallel α-helices very closely resembling the peptide cleft in MHC I molecules. In nature, FcRn binds and transports IgG across the placental syncytiotrophoblast from maternal circulation to fetal circulation and protects IgG from degradation in adults. In addition to homeostasis, FcRn controls transcytosis of IgG in tissues. FcRn is localized in epithelial cells, endothelial cells and hepatocytes.

Studies have shown that albumin binds FcRn to form a tri-molecular complex with IgG. Both albumin and IgG bind noncooperatively to distinct sites on FcRn. Binding of human FcRn to Sepharose-HSA and Sepharose-hIgG is pH dependent, being maximal at pH 5.0 and nil at pH 7.0 through pH 8. The observation that FcRn binds albumin in the same pH dependent fashion as it binds IgG suggests that the mechanism by which albumin interacts with FcRn and thus is protected from degradation is identical to that of IgG, and mediated via a similarly pH-sensitive interaction with FcRn. FcRn and albumin interact via the D-III domain of albumin in a pH-dependent manner, on a site distinct from the IgG binding site.

The Fn fusions of the present invention also include Fn-FcRn fusion proteins or Fn-anti-FcRn fusion molecules. In one embodiment, the Fn fusion is an Fn-anti-FcRn fusion molecule in which an anti-FcRn fusion molecule can bind to the neonatal FcR receptor (FcRn) with high affinity at acidic pH (e.g. pH 6.0) and low affinity at neutral pH (e.g. pH 7.4) similar to IgG binding to FcRn. The half-life of an Fn-anti-FcRn fusion increased in vivo thereby providing improved therapeutic utility.

Methods for fusing molecules to an Fc domain, e.g., the Fc domain of IgG1, are known in the art (see, e.g., U.S. Pat. No. 5,428,130). Such fusions have increased circulating half-lives, due to the ability of Fc to bind to FcRn, which serves a critical function in IgG homeostasis, protecting molecules bound to it from catabolism. (See E.g., US 20070269422).

Other fusions include a fibronectin-based binding molecule fused to human serum albumin (HSA or HA). Human serum albumin, a protein of 585 amino acids in its mature form, is responsible for a significant proportion of the osmotic pressure of serum and also functions as a carrier of endogenous and exogenous ligands. The role of albumin as a carrier molecule and its inert nature are desirable properties for use as a carrier and transporter of polypeptides in vivo. The use of albumin as a component of an albumin fusion protein as a carrier for various proteins has been suggested in WO 93/15199, WO 93/15200, and EP 413 622. The use of N-terminal fragments of HSA for fusions to polypeptides has also been proposed (EP 399 666). Accordingly, by genetically or chemically fusing or conjugating the molecules of the present invention to albumin, or a fragment (portion) or variant of albumin or a molecule capable of binding HSA (an “anti-HSA binder”) that is sufficient to stabilize the protein and/or its activity, the molecule is stabilized to extend the shelf-life, and/or to retain the molecule's activity for extended periods of time in solution, in vitro and/or in vivo.

Fusion of albumin to another protein may be achieved by genetic manipulation, such that the DNA coding for HSA, or a fragment thereof, is joined to the DNA coding for the protein. A suitable host is then transformed or transfected with the fused nucleotide sequences, so arranged on a suitable plasmid as to express a fusion polypeptide. The expression may be effected in vitro from, for example, prokaryotic or eukaryotic cells, or in vivo e.g. from a transgenic organism. Additional methods pertaining to HSA fusions can be found, for example, in WO 2001077137 and WO 200306007, incorporated herein by reference. In a specific embodiment, the expression of the fusion protein is performed in mammalian cell lines. Examples of mammalian cells include, but are not limited to, Human Embryonic Kidney cells (e.g. HEK Freestyle, HEK293, HEK293T); Chinese Hamster Ovary cells (e.g. CHO); Hamster Kidney cells (e.g. BHK); Human embryonic retinal cells (e.g PERC6); Mouse myeloma (Sp/20); Hybrid of HEK293 and a human B cell line (e.g. HKB11); Cervical cancer cells (e.g HeLa); and Monkey kidney cells (e.g. COS). In one embodiment, the mammalian cells are CHO cells.

Other fusions of the present invention include linking a fibronectin-based binding molecule to another functional molecule, e.g., another peptide or protein (e.g., an antibody or ligand for a receptor) to generate a “bispecific molecule.” A bispecific molecule binds to at least two different binding sites or at least two different target molecules, e.g., the binding site targeted by the fibronectin molecule and an anti-HSA binder, said anti-HSA binder being either derived from a fibronectin-based molecule (as described above) or from other non-fibronectin scaffold, and for example, from a single domain antibody (see, e.g., WO2004041865 (Ablynx) and EP1517921 (Domantis)). The fibronectin-based binding molecule of the invention may also be derivatized or linked to more than one other functional molecule to generate multispecific molecules that bind to more than two different binding sites on the same target molecule, and/or two separate binding sites on two different target molecules and various permutations thereof. In one embodiment, a Fn3 based binding multispecific molecule can comprise for example, at least two Fn3 domains linked together and conjugated to a half-life extension moiety such as HSA, such that each of the Fn3 domains binds to different sites of the same therapeutic target, e.g., different sites on TNF. In another embodiment, a Fn3 based binding multispecific molecule can comprise for example, at least two Fn3 domains linked together and conjugated to a half-life extension moiety such as HSA, such that each of the Fn3 domains binds to different therapeutic targets, e.g., the first Fn3 domain bind to Her3 and the second Fn3 domain binds to Her2. In yet another embodiment, a Fn3 based binding multispecific molecule can comprise for example, at least two Fn3 domains linked together and conjugated to a half-life extension moiety such as HSA, such that each of the Fn3 domains binds to different sites on different therapeutic targets, e.g., the first Fn3 domain binds to site 1 of Her3, the second Fn3 domain binds to site 2 of Her 3, the third Fn3 domain binds to site 1 of Her2 and the fourth Fn3 domain binds to site 2 of Her2, and various permutations thereof. Such multispecific molecules are also intended to be encompassed by the term “bispecific molecule” as used herein.

The bispecific molecules of the present invention can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohexane-1-carboxylate (sulfo-SMCC) (see e.g., Karpovsky et al. (1984) J. Exp. Med. 160:1686; Liu, M A et al. (1985) Proc. Natl. Acad. Sci. U.S.A 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

If the binding specificities include more than one antibody (e.g., in a multispecific construct), conjugation can be achieved via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bispecific molecules to their specific targets can be confirmed by various assays, for example, the fusion can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ-counter or a scintillation counter or by autoradiography.

Other fusions of the present invention include linking a fibronectin-based binding molecule to a tag (e.g., biotin) or a chemical (e.g., an immunotoxin or chemotherapeutic agent). Such chemicals include cytotoxic agent which is any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Other examples of therapeutic cytotoxins that can be conjugated to fibronectin-based binding molecule of the invention include duocarmycins, calicheamicins, maytansines and auristatins, and derivatives thereof.

Cytoxins can be conjugated to the fibronectin-based binding molecules of the invention using linker technology available in the art. Examples of linker types that have been used to conjugate a cytotoxin include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents, see also Saito, G. et al. (2003)Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Fibronectin-based binding molecules of the present invention also can be conjugated to a radioactive isotope to generate cytotoxic radiopharmaceuticals, also referred to as radioimmunoconjugates. Examples of radioactive isotopes that can be conjugated to fibronectin-based binding molecules for use diagnostically or therapeutically include, but are not limited to, iodine¹³¹, indium¹¹¹, yttrium⁹⁰ and lutetium¹⁷⁷. Methods for preparing radioimmunoconjugates are established in the art. Examples of antibody-based radioimmunoconjugates are commercially available, including ibritumomab, tiuxetan, and tositumomab, and similar methods can be used to prepare radioimmunoconjugates using the molecules of the invention.

The Fn fusions of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety are well known and can be applied to the molecules of the present invention, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### Chemical Modifications

In another aspect, the invention provides fibronectin-based binding molecules that are modified by pegylation, for example, to increase the biological (e.g., serum) half life of the molecule. To pegylate a molecule, the molecule, or fragment thereof, typically is reacted with a polyethylene glycol (PEG) moiety, such as a reactive ester or aldehyde derivative of PEG, under conditions in which one or more PEG groups become attached to the molecule. The term “PEGylation moiety”, “polyethylene glycol moiety”, or “PEG moiety” includes a polyalkylene glycol compound or a derivative thereof, with or without coupling agents or derviatization with coupling or activating moieties (e.g., with thiol, triflate, tresylate, azirdine, oxirane, or preferably with a maleimide moiety, e.g., PEG-maleimide). Other appropriate polyalkylene glycol compounds include, but are not limited to, maleimido monomethoxy PEG, activated PEG polypropylene glycol, but also charged or neutral polymers of the following types: dextran, colominic acids, or other carbohydrate based polymers, polymers of amino acids, and biotin derivatives.

The choice of the suitable functional group for the PEG derivative is based on the type of available reactive group on the molecule or molecule that will be coupled to the PEG. For proteins, typical reactive amino acids include lysine, cysteine, histidine, arginine, aspartic acid, glutamic acid, serine, threonine, tyrosine. The N-terminal amino group and the C-terminal carboxylic acid can also be used.

Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. Methods for pegylating proteins are known in the art and can be applied to the present invention. See for example, WO 2005056764, U.S. Pat. No. 7,045,337, U.S. Pat. No. 7,083,970, U.S. Pat. No. 6,927,042, EP 0 154 316 by Nishimura et al. and EP 0 401 384 by Ishikawa et al. Fibronectin-based binding molecules can be engineered to include at least one cysteine amino acid or at least one non-natural amino acid to facilitate pegylation.

Fibronectin-based binding molecules of the present invention also can be modified by hesylation, which utilizes hydroxyethyl starch (“HES”) derivatives linked to drug substances in order to modify the drug characteristics. HES is a modified natural polymer derived from waxy maize starch which is metabolized by the body's enzymes. This modification enables the prolongation of the circulation half-life by increasing the stability of the molecule, as well as by reducing renal clearance, resulting in an increased biological activity. Furthermore, HESylation potentially alters the immunogenicity or allergenicity. By varying different parameters, such as the molecular weight of HES, a wide range of HES drug conjugates can be customized.

DE 196 28 705 and DE 101 29 369 describe possible methods for carrying out the coupling of hydroxyethyl starch in anhydrous dimethyl sulfoxide (DMSO) via the corresponding aldonolactone of hydroxyethyl starch with free amino groups of hemoglobin and amphotericin B, respectively. Since it is often not possible to use anhydrous, aprotic solvents specifically in the case of proteins, either for solubility reasons or else on the grounds of denaturation of the proteins, coupling methods with HES in an aqueous medium are also available. For example, coupling of hydroxyethyl starch which has been selectively oxidized at the reducing end of the chain to the aldonic acid is possible through the mediation of water-soluble carbodiimide EDC (1-ethyl-3-(3-dimethyl-aminopropyl)carbodiimide) (PCT/EP 02/02928). Additional hesylation methods which can be applied to the present invention are described, for example, in U.S. 20070134197, U.S. 20060258607, U.S. 20060217293, U.S. 20060100176, and U.S. 20060052342.

Fibronectin-based binding molecules of the invention also can be modified via sugar residues. Methods for modifying sugar residues of proteins or glycosylating proteins are known in the art (see, for example, Borman (2006) Chem. and Eng. News 84(36):13-22 and Borman (2007) Chem. and Eng. News 85:19-20) and can be applied to the molecules of the present invention. Such carbohydrate modifications can also be accomplished by; for example, altering one or more sites of glycosylation within the fibronectin-based binding molecule sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, a Fibronectin-based binding molecules of the invention can be made that has an altered type of glycosylation, such as a hypofucosylated pattern having reduced amounts of fucosyl residues or an fibronectin-based binding molecule having increased bisecting GlcNac structures. Such carbohydrate modifications can be accomplished by, for example, expressing the fibronectin-based binding molecule in a host cell with altered glycosylation machinery. Cells with altered glycosylation machinery have been described in the art and can be used as host cells in which to express recombinant Fibronectin-based binding molecules of the invention to thereby produce Fibronectin-based binding molecules of the invention with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180). Methods to produce polypeptides with human-like glycosylation patterns have also been described by EP1297172B1 and other patent families originating from Glycofi.

### Amino Acid/Nucleotide Modifications

Fibronectin-based binding molecules of the invention having one or more amino acid or nucleotide modifications (e.g., alterations) can be generated by a variety of known methods. Such modified molecules can, for example, be produced by recombinant methods. Moreover, because of the degeneracy of the genetic code, a variety of nucleic acid sequences can be used to encode each desired molecule.

Exemplary art recognized methods for making a nucleic acid molecule encoding an amino acid sequence variant of a starting molecule include, but are not limited to, preparation by site-directed (or oligonucleotide-mediated) mutagenesis, PCR mutagenesis, and cassette mutagenesis of an earlier prepared DNA encoding the molecule.

Site-directed mutagenesis is a preferred method for preparing substitution variants. This technique is well known in the art (see, e.g., Carter et al. Nucleic Acids Res. 13:4431-4443 (1985) and Kunkel et al., Proc. Natl. Acad. Sci. U.S.A 82:488 (1987)). Briefly, in carrying out site-directed mutagenesis of DNA, the parent DNA is altered by first hybridizing an oligonucleotide encoding the desired mutation to a single strand of such parent DNA. After hybridization, a DNA polymerase is used to synthesize an entire second strand, using the hybridized oligonucleotide as a primer, and using the single strand of the parent DNA as a template. Thus, the oligonucleotide encoding the desired mutation is incorporated in the resulting double-stranded DNA.

PCR mutagenesis is also suitable for making amino acid sequence variants of the starting molecule. See Higuchi, in PCR Protocols, pp. 177-183 (Academic Press, 1990); and Vallette et al., Nuc. Acids Res. 17:723-733 (1989). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., Gene 34:315-323 (1985). The starting material is the plasmid (or other vector) comprising the starting polypeptide DNA to be mutated. The codon(s) in the parent DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a polypeptide variant of the molecule can be determined, and a nucleic acid sequence encoding such amino acid sequence variant can be generated synthetically.

It will be understood by one of ordinary skill in the art that the fibronectin-based binding molecules of the invention may further be modified such that they vary in amino acid sequence (e.g., from wild-type), but not in desired activity. For example, additional nucleotide substitutions leading to amino acid substitutions at “non-essential” amino acid residues may be made to the protein For example, a nonessential amino acid residue in a molecule may be replaced with another amino acid residue from the same side chain family. In another embodiment, a string of amino acids can be replaced with a structurally similar string that differs in order and/or composition of side chain family members, i.e., a conservative substitutions, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Aside from amino acid substitutions, the present invention contemplates other modifications of the starting molecule amino acid sequence in order to generate functionally equivalent molecules. For example, one may delete one or more amino acid residues. Generally, no more than one to about ten residues will be deleted according to this embodiment of the invention. The fibronectin molecules herein comprising one or more amino acid deletions will preferably retain at least about 80%, and preferably at least about 90%, and most preferably at least about 95%, of the starting polypeptide molecule.

One may also make amino acid insertion variants, which retain the original fibronectin-molecule functionality. For example, one may introduce at least one amino acid residue (e.g. one to two amino acid residues and generally no more than ten residues) into the molecule. In another embodiment amino acid modifications may be combined within a single fibronectin molecule.

In one embodiment, amino acid substitutions are performed on fibronectin type 3 domain to include cysteine or other non-natural amino acid suitable for conjugating a moiety to the fibronectin-based binding molecule using well-known conjugating methods. In particular, the invention relates to specific amino acid variants of fibronectin-based binding molecule with Fn3 scaffold, wherein one or more serine amino acid residues are substituted by cysteine or a non-natural amino acid. Serine amino acid residues that can substituted include, but are not limited to Ser 17, Ser 21, Ser 43, Ser 60, and Ser 89. Other amino acid positions of the Fn3 scaffold that can be substituted include, but are not limited to, Val11, Leu19, Thr58 and Thr71. Non-naturally occurring amino acids can be substituted into the Fn3 scaffold using, for example, Ambrex technology (See e.g., U.S. Pat. Nos. 7,045,337; 7,083,970).

### Screening Assays for Identifying Improved Fibronectin-Based Binding Molecules

A variety of screening assays can be employed to identify improved fibronectin-based binding molecules of the invention. In one embodiment, fibronectin-based binding molecules are screened for improved binding affinity to a desired antigen. Any in vitro or in vivo screening method that selects for improved binding to the desired antigen is contemplated.

In another embodiment fibronectin-based binding molecules are displayed on the surface of a cell, virus or bacteriophage and subject to selection using immobilized antigen. Suitable methods of screening are described in U.S. Pat. Nos. 7,063,943; 6,699,658; 7,063,943 and 5,866,344. Such surface display may require the creation of fusion proteins of the fibronectin-based binding molecules with a suitable protein normally present on the outer surface of a cell, virus or bacteriophage. Suitable proteins from which to make such fusions are well know in the art.

In another embodiment fibronectin-based binding molecules are screened using an in vitro phenotype-genotype linked display such as ribosome or polysome display. Such methods of “molecular evolution” are well known in the art (see for example U.S. Pat. Nos. 6,194,550 and 7,195,880).

Screening methods employed in the invention may require that one or more amino acid mutations are introduced into the fibronectin-based binding molecules. Any art recognized methods of mutagenesis are contemplated. In one embodiment, a library of fibronectin-based binding molecules is created in which one or more amino acids in the Fn3 scaffold or the grafted CDRs are randomly mutated. In another embodiment, a library of fibronectin-based binding molecules is created in which one or more amino acids in the Fn3 scaffold or the grafted CDRs are mutated to one or more predetermined amino acid.

Screening methods employed in the invention may also require that the stringency of the antigen-binding screening assay is increased to select for fibronectin-based binding molecules with improved affinity for antigen. Art recognized methods for increasing the stringency of a protein-protein interaction assay can be used here. In one embodiment, one or more of the assay conditions are varied (for example, the salt concentration of the assay buffer) to reduce the affinity of the fibronectin-based binding molecules for the desired antigen. In another embodiment, the length of time permitted for the fibronectin-based binding molecules to bind to the desired antigen is reduced. In another embodiment, a competitive binding step is added to the protein-protein interaction assay. For example, the fibronectin-based binding molecules are first allowed to bind to a desired immobilized antigen. A specific concentration of non-immobilized antigen is then added which serves to compete for binding with the immobilized antigen such that the fibronectin-based binding molecules with the lowest affinity for antigen are eluted from the immobilized antigen resulting in selection of fibronectin-based binding molecules with improved antigen binding affinity. The stringency of the assay conditions can be further increased by increasing the concentration of non-immobilized antigen is added to the assay.

Screening methods of the invention may also require multiple rounds of selection to enrich for one or more fibronectin-based binding molecules with improved antigen binding. In one embodiment, at each round of selection further amino acid mutation are introduce into the fibronectin-based binding molecules. In another embodiment, at each round of selection the stringency of binding to the desired antigen is increased to select for fibronectin-based binding molecules with increased affinity for antigen.

### Methods of Manufacture

The fibronectin-based binding molecules of the invention are typically produced by recombinant expression. Nucleic acids encoding the molecules are inserted into expression vectors. The DNA segments encoding the molecules are operably linked to control sequences in the expression vector(s) that ensure their expression. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting fibronectin-based binding molecule.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362).

E. coli is one prokaryotic host particularly useful for cloning the polynucleotides (e.g., DNA sequences) of the present invention. Other microbial hosts suitable for use include bacilli, such as Bacillus subtilis, and other enterobacteriaceae, such as Salmonella, Serratia, and various Pseudomonas species.

Other microbes, such as yeast, are also useful for expression. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

In addition to microorganisms, mammalian tissue culture may also be used to express and produce the polypeptides of the present invention (e.g., polynucleotides encoding immunoglobulins or fragments thereof). See Winnacker, From Genes to Clones, VCH Publishers, N.Y., N.Y. (1987). Eukaryotic cells are actually preferred, because a number of suitable host cell lines capable of secreting heterologous proteins (e.g., intact immunoglobulins) have been developed in the art, and include CHO cell lines, various COS cell lines, HeLa cells, 293 cells, myeloma cell lines, transformed B-cells, and hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

Alternatively, coding sequences can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

The vectors containing the polynucleotide sequences of interest and expression control sequences can be transferred into the host cell by well-known methods, which vary depending on the type of cellular host. For example, chemically competent prokaryotic cells may be briefly heat-shocked, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed, the fibronectin-based binding molecules of the present invention can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure molecules of at least about 90 to 95% homogeneity are preferred, and 98 to 99% or more homogeneity most preferred, for pharmaceutical uses.

### Compositions

The fibronectin-based binding molecules (and variants, fusions, and conjugates thereof) of the present invention have in vitro and in vivo diagnostic and therapeutic utilities. Accordingly, the present invention also provides compositions, e.g., a pharmaceutical composition, containing one or a combination of fibronectin-based binding molecules (or variants, fusions, and conjugates thereof), formulated together with a pharmaceutically acceptable carrier. Pharmaceutical compositions of the invention also can be administered in combination therapy, i.e., combined with other agents. For example, the combination therapy can include a composition of the present invention with at least one or more additional therapeutic agents, such as anti-inflammatory agents, anti-cancer agents, and chemotherapeutic agents.

The pharmaceutical compositions of the invention can also be administered in conjunction with radiation therapy. Co-administration with other fibronectin-based molecules are also encompassed by the invention.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition of the present invention can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the Fibronectin-based binding molecule of the invention may be administered once or twice weekly by subcutaneous injection or once or twice monthly by subcutaneous injection. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.001 percent to about ninety percent of active ingredient, preferably from about 0.005 percent to about 70 percent, most preferably from about 0.01 percent to about 30 percent.

Formulations of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a compositions of the invention will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of therapeutic compositions may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. No. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the molecules of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the invention, the therapeutic compounds of the invention are formulated in liposomes; in a more preferred embodiment, the liposomes include a targeting moiety. In a most preferred embodiment, the therapeutic compounds in the liposomes are delivered by bolus injection to a site proximal to the tumor or infection. The composition must be fluid to the extent that easy syringability exists. It must be stable under the conditions of manufacture and storage and must be preserved against the contaminating action of microorganisms such as bacteria and fungi.

In a further embodiment, the molecules of the invention can be formulated to prevent or reduce the transport across the placenta. This can be done by methods known in the art, e.g., by PEGylation of the fibronectin-based binding molecule. Further references can be made to “Cunningham-Rundles C, Zhuo Z, Griffith B, Keenan J. (1992) Biological activities of polyethylene-glycol immunoglobulin conjugates. Resistance to enzymatic degradation. J Immunol Methods. 152:177-190; and to “Landor M. (1995) Maternal-fetal transfer of immunoglobulins, Ann Allergy Asthma Immunol 74:279-283. This is particularly relevant when the fibronectin-based binding molecule are used for treating or preventing recurrent spontaneous abortion.

The ability of a compound to inhibit cancer can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

The composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound may be orally administered, for example, with an inert diluent or an assimilable edible carrier.

### Therapeutic and Diagnostic Applications

The fibronectin-based binding molecules described herein may be constructed to bind any antigen of interest and may be modified to have increased stability and half-life, as well as additional functional moieties. Accordingly, these molecules may be employed in place of antibodies in all areas in which antibodies are used, including in the research, therapeutic, and diagnostic fields. In addition, because these molecules possess solubility and stability properties superior to antibodies, the antibody mimics described herein may also be used under conditions which would destroy or inactivate antibody molecules.

For example, these molecules can be administered to cells in culture, e.g. in vitro or ex vivo, or in a subject, e.g., in vivo, to treat, prevent or diagnose a variety of disorders. The term “subject” as used herein in intended to includes human and non-human animals. Non-human animals includes all vertebrates, e.g., mammals and non-mammals, such as non-human primates, sheep, dogs, cats, cows, horses, chickens, amphibians, and reptiles. When the fibronectin molecules are administered together with another agent, the two can be administered in either order or simultaneously.

In one embodiment, the fibronectin-based binding molecules (and variants, fusions, and conjugates thereof) of the invention can be used to detect levels of the target bound by the molecule and/or the targets bound by a bispecific/multispecific fibronectin-based binding molecule. This can be achieved, for example, by contacting a sample (such as an in vitro sample) and a control sample with the molecule under conditions that allow for the formation of a complex between the molecule and the target(s). Any complexes formed between the molecule and the target(s) are detected and compared in the sample and the control. For example, standard detection methods, well-known in the art, such as ELISA, FACS, and flow cytometric assays, can be performed using the compositions of the invention.

Also within the scope of the invention are kits comprising the compositions (e.g., fibronectin-based binding molecules, variants, fusions, and conjugates thereof) of the invention and instructions for use. The kit can further contain a least one additional reagent, or one or more additional fibronectin molecules of the invention (e.g., an antibody having a complementary activity which binds to an epitope on the target antigen distinct from the first molecule). Kits typically include a label indicating the intended use of the contents of the kit. The term label includes any writing, or recorded material supplied on or with the kit, or which otherwise accompanies the kit.

As described above, the molecules of the present invention may be employed in all areas of the research, therapeutic, and diagnostic fields. Exemplary diseases/disorders which can be treated using the fibronectin-based binding molecules of the present invention (and variants, fusions, and conjugates thereof) include, but are not limited to, autoimmune diseases, inflammation, cancer, infectious diseases, cardiovascular diseases, gastrointestinal diseases, respiratory diseases, metabolic diseases, musculoskeletal diseases, neurodegenerative diseases, psychiatric diseases, opthalmic diseases, hyperplasia, diabetic retinopathy, macular degeneration, inflammatory bowel disease, Crohn's disease, ulcerative colitis, rheumatoid arthritis, diabetes, sarcoidosis, asthma, edema, pulmonary hypertension, psoriasis, corneal graft rejection, neovascular glaucoma, Osler-Webber Syndrome, myocardial angiogenesis, plaque neovascularization, restenosis, neointima formation after vascular trauma, telangiectasia, hemophiliac joints, angiofibroma, fibrosis associated with chronic inflammation, lung fibrosis, amyloidosis, Alzheimer's disease, organ transplant rejection, deep venous thrombosis or wound granulation.

In one embodiment, the molecules of the invention can be used to treat autoimmune disease, such as acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, juvenile diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis (i.e., Graves' disease), scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis or fibrosing alveolitis.

In another embodiment, the molecules of the invention can be used to treat cancer. Exemplary types of tumors that may be targeted include acute lymphocytic leukemia, acute myelogenous leukemia, biliary cancer, breast cancer, cervical cancer, chronic lymphocytic leukemia, chronic myelogenous leukemia, colorectal cancer, endometrial cancer, esophageal cancer, gastric cancer, head and neck cancers, Hodgkin's lymphoma, lung cancer, medullary thyroid cancer, non-Hodgkin's lymphoma, multiple myeloma, renal cancer, ovarian cancer, pancreatic cancer, melanoma, liver cancer, prostate cancer, glial and other brain and spinal cord tumors, and urinary bladder cancer.

In another embodiment, the molecules of the invention can be used to treat infection with pathogenic organisms, such as bacteria, viruses, fungi, or unicellular parasites. Exemplary fungi that may be treated include Microsporum, Trichophyton, Epidermophyton, Sporothrix schenckii, Cryptococcus neoformans, Coccidioides immitis, Histoplasma capsulatum, Blastomyces dermatitidis or Candida albican. Exemplary viruses include human immunodeficiency virus (HIV), herpes virus, cytomegalovirus, rabies virus, influenza virus, human papilloma virus, hepatitis B virus, hepatitis C virus, Sendai virus, feline leukemia virus, Reo virus, polio virus, human serum parvo-like virus, simian virus 40, respiratory syncytial virus, mouse mammary tumor virus, Varicella-Zoster virus, Dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus or blue tongue virus. Exemplary bacteria include Bacillus anthracis, Streptococcus agalactiae, Legionella pneumophilia, Streptococcus pyogenes, Escherichia coli, Neisseria gonorrhoeae, Neisseria meningitidis, Pneumococcus spp., Hemophilis influenzae B, Treponema pallidum, Lyme disease spirochetes, Pseudomonas aeruginosa, Mycobacterium leprae, Brucella abortus, Mycobacterium tuberculosis or a Mycoplasma. Exemplary parasites include Giardia lamblia, Giardia spp., Pneumocystis carinii, Toxoplasma gondii, Cryptospordium spp., Acanthamoeba spp., Naegleria spp., Leishmania spp., Balantidium coli, Trypanosoma evansi, Trypanosoma spp., Dientamoeba fragilis, Trichomonas vaginalis, Trichmonas spp. Entamoeba spp. Dientamoeba spp. Babesia spp., Plasmodium falciparum, Isospora spp., Toxoplasma spp. Enterocytozoon spp., Pneumocystis spp. and Balantidium spp.

### Therapeutic and Diagnostic Applications

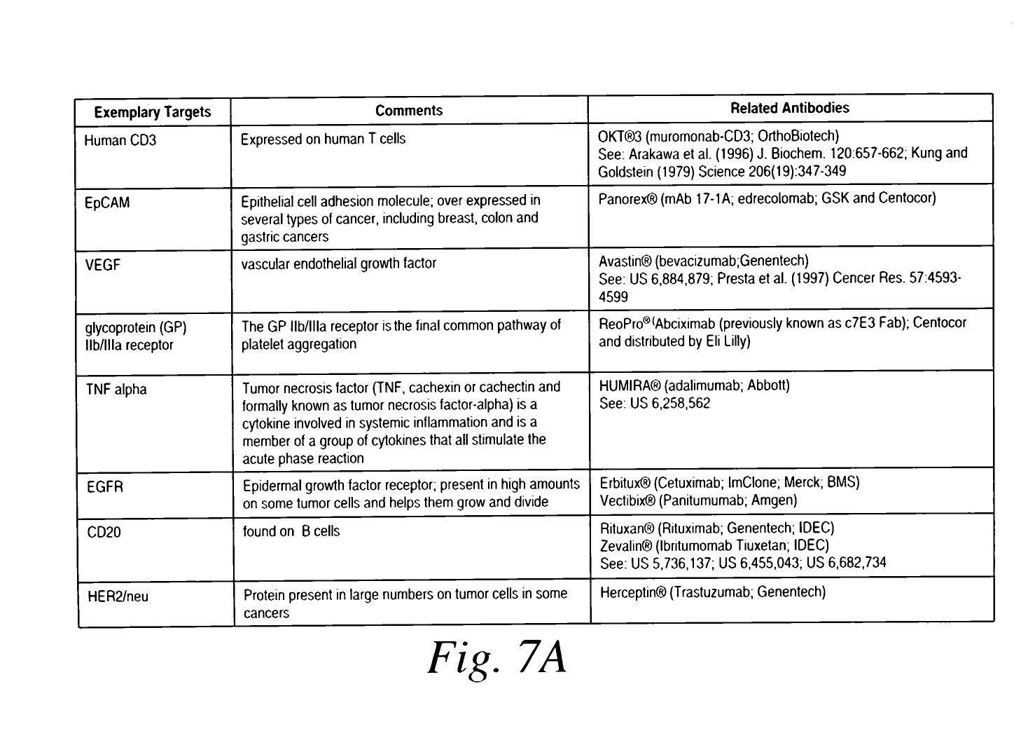

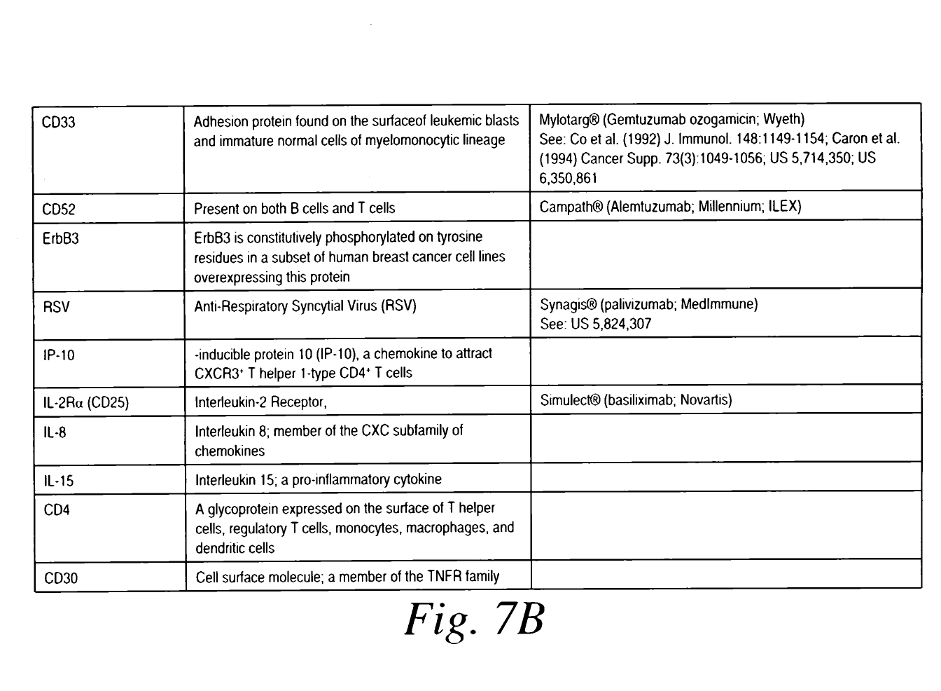

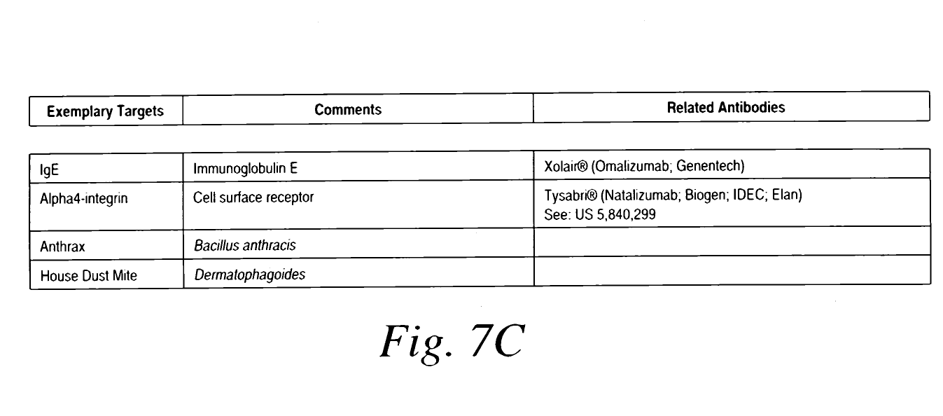

The fibronectin-based binding molecules described herein may be constructed to bind any antigen or target of interest. Such targets include, but are not limited to, cluster domains, cell receptors, cell receptor ligands, growth factors, interleukins, protein allergens, bacteria, or viruses (see, for example, FIG. 7A-C). The fibronectin-based binding molecules described herein may also be modified to have increased stability and half-life, as well as additional functional moieties. Accordingly, these molecules may be employed in place of antibodies in all areas in which antibodies are used, including in the research, therapeutic, and diagnostic fields. In addition, because these molecules possess solubility and stability properties superior to antibodies, the antibody mimics described herein may also be used under conditions which would destroy or inactivate antibody molecules.

The present invention is further illustrated by the following examples which should not be construed as further limiting. The contents of all figures and all references, patents and published patent applications cited throughout this application are expressly incorporated herein by reference.

### Exemplification

Throughout the examples, the following materials and methods were used unless otherwise stated.

### Materials and Methods

In general, the practice of the present invention employs, unless otherwise indicated, conventional techniques of chemistry, molecular biology, recombinant DNA technology, immunology (especially, e.g., antibody technology), and standard techniques in polypeptide preparation. See, e.g., Sambrook, Fritsch and Maniatis, Molecular Cloning Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); and Current Protocols in Molecular Biology, eds. Ausubel et al., John Wiley and Sons (1992). Other methods, techniques, and sequences suitable for use in carrying out the present invention are found in U.S. Pat. Nos. 7,153,661; 7,119,171; 7,078,490; 6,703,199; 6,673,901; and 6,462,189.

### Sequences

The following sequences were used throughout.

### Wildtype Fn3 Sequence

(SEQ ID NO: 1)
VSDVPRDLEVVAATPTSLLISWDAPAVTVRYYRITYGETGGNSPVQEFTV
PGSKSTATISGLKPGVDYTITVYAVTGRGDSPASSKPISINYRT

### Wildtype Fn3 Sequence (RGD to RGA)

(SEQ ID NO: 2)
VSDVPRDLEVVAATPTSLLISWDAPAVTVRYYRITYGETGGNSPVQEFTV
PGSKSTATISGLKPGVDYTITVYAVTGRGASPASSKPISINYRT

### TNF-Binding Fn3 Sequence

(SEQ ID NO: 3)
VSDVPRDLEVVAATPTSRLISWNRSGLQSRYYRITYGETGGNSPVQEFTV
PPWASIATISGLKPGVDYTITVYAVTDKSDTYKYDDPISINYRT

### TNF-Binding Fn3 (R18L and I56T)

(SEQ ID NO: 4)
VSDVPRDLEVVAATPTSLLISWNRSGLQSRYYRITYGETGGNSPVQEFTV
PPWASTATISGLKPGVDYTITVYAVTDKSDTYKYDDPISINYRT

### VEGFR-Binding Fn3

(SEQ ID NO: 76)
GEVVAATPTSLLISWRHPHFPTRYYRITYGETGGNSPVQEFTVPLQPPTA
TISGLKPGVDYTITVYAVTDGRNGRLLSIPISINYRT

dsbA Signal Sequence

| | |
|---|---|
| MKKIWLALAGLVLAFSASA | (SEQ ID NO: 5) |

### CD33 Signal Sequence+TNF-Binding Fn3 Sequence

(SEQ ID NO: 6)
MPLLLLLPLLWAGALAVSDVPRDLEVVAATPTSRLISWNRSGLQSRYYRI
TYGETGGNSPVQEFTVPPWASIATISGLKPGVDYTITVYAVTDKSDTYKY
DDPISINYRT

### CD33 Signal Sequence+TNF-Binding Fn3 (R18L and I56T)

(SEQ ID NO: 7)
MPLLLLLPLLWAGALAVSDVPRDLEVVAATPTSLLISWNRSGLQSRYYRI
TYGETGGNSPVQEFTVPPWASTATISGLKPGVDYTITVYAVTDKSDTYKY
DDPISINYRT

### CD33 Signal Sequence+Wildtype Fn3

(SEQ ID NO: 8)
MPLLLLLPLLWAGALAVSDVPRDLEVVAATPTSLLISWDAPAVTVRYYRI
TYGETGGNSPVQEFTVPGSKSTATISGLKPGVDYTITVYAVTGRGDSPAS
SKPISINYRT

### CD33 Signal Sequence+Wildtype Fn3 (RGD to RGA)

(SEQ ID NO: 9)
MPLLLLLPLLWAGALAVSDVPRDLEVVAATPTSLLISWDAPAVTVRYYRI
TYGETGGNSPVQEFTVPGSKSTATISGLKPGVDYTITVYAVTGRGASPAS
SKPISINYRT

### CD33 Signal Sequence+VEGFR-Binding Fn3

(SEQ ID NO: 77)
MPLLLLLPLLWAGALAGEVVAATPTSLLISWRHPHFPTRYYRITYGETGG
NSPVQEFTVPLQPPTATISGLKPGVDYTITVYAVTDGRNGRLLSIPISIN
YRT

### TNF-Binding Nanobody

(SEQ ID NO: 10)
QVQLVESGGGLVQPGGSLRLSCAASGFTFSDYWMYWVRQAPGKGLEWVSE
INTNGLITKYPDSVKGRFTISRDNAKNTLYLQMNSLKPEDTALYYCARSP
SGFNRGQGTQVTVSS

### TNF-Binding Single Domain Antibody

(SEQ ID NO: 11)
DIQMTQSPSSLSASVGDRVTITCRASQAIDSYLHWYQQKPGKAPKLLIYS
ASNLETGVPSRFSGSGSGTDFTLTISSLLPEDFATYYCQQVVWRPFTFGQ
GTKVEIKR

### Anti-HSA Binder

(SEQ ID NO: 12)
EVQLLESGGGLVQPGGSLRLSCAASGFTFDEYNMSWVRQAPGKGLEW
VSTILPHGDRTYYADSVKGRFTISRDNSKNTLYLQMNSLRAEDTAVY
YCAKQDPLYRFDYWGQGTLVTVSS_

### Anti-MSA Binder

(SEQ ID NO: 13)
DIQMTQSPSSLSASVGDRVTITCRASQSIIKHLKWYQQKPGKAPKLL
IYGASRLQSGVPSRFSGSGSGTDFTLTISSLQPEDFATYYCQQGARW
PQTFGQGTKVEIKR

### Anti-RSA Binder

(SEQ ID NO: 78)
DIQMTQSPSSLSASVGDRVTITCRASQSISSYLNWYQQKPGKAPKLL
IYRNSPLQSGVPSRFSGSGSGTDFTLTISSLQPEDFATYYCQQTYRV
PPTFGQGTKVEIKR

### Human Serum Albumin (HSA)

(SEQ ID NO: 14)
DAHKSEVAHRFKDLGEENFKALVLIAFAQYLQQCPFEDHVKLVNEVT
EFAKTCVADESAENCDKSLHTLFGDKLCTVATLRETYGEMADCCAKQ
EPERNECFLQHKDDNPNLPRLVRPEVDVMCTAFHDNEETFLKKYLYE
IARRHPYFYAPELLFFAKRYKAAFTECCQAADKAACLLPKLDELRDE
GKASSAKQRLKCASLQKFGERAFKAWAVARLSQRFPKAEFAEVSKLV
TDLTKVHTECCHGDLLECADDRADLAKYICENQDSISSKLKECCEKP
LLEKSHCIAEVENDEMPADLPSLAADFVESKDVCKNYAEAKDVFLGM
FLYEYARRHPDYSVVLLLRLAKTYETTLEKCCAAADPHECYAKVFDE
FKPLVEEPQNLIKQNCELFEQLGEYKFQNALLVRYTKKVPQVSTPTL
VEVSRNLGKVGSKCCKHPEAKRMPCAEDYLSVVLNQLCVLHEKTPVS
DRVTKCCTESLVNRRPCFSALEVDETYVPKEFNAETFTFHADICTLS
EKERQIKKQTALVELVKHKPKATKEQLKAVMDDFAAFVEKCCKADDK
ETCFAEEGKKLVAASQAALGL

### Rat Serum Albumin (RSA)

(SEQ ID NO: 79)
EAHKSEIAHRFKDLGEQHFKGLVLIAFSQYLQKCPYEEHIKLVQEVT
DFAKTCVADENAENCDKSIHTLFGDKLCAIPKLRDNYGELADCCAKQ
EPERNECFLQHKDDNPNLPPFQRPEAEAMCTSFQENPTSFLGHYLHE
VARRHPYFYAPELLYYAEKYNEVLTQCCTESDKAACLTPKLDAVKEK
ALVAAVRQRMKCSSMQRFGERAFKAWAVARMSQRFPNAEFAEITKLA
TDVTKINKECCHGDLLECADDRAELAKYMCENQATISSKLQACCDKP
VLQKSQCLAEIEHDNIPADLPSIAADFVEDKEVCKNYAEAKDVFLGT
FLYEYSRRHPDYSVSLLLRLAKKYEATLEKCCAEGDPPACYGTVLAE
FQPLVEEPKNLVKTNCELYEKLGEYGFQNAVLVRYTQKAPQVSTPTL
VEAARNLGRVGTKCCTLPEAQRLPCVEDYLSAILNRLCVLHEKTPVS
EKVTKCCSGSLVERRPCFSALTVDETYVPKEFKAETFTFHSDICTLP
DKEKQIKKQTALAELVKHKPKATEDQLKTVMGDFAQFVDKCCKAADK
DNCFATEGPNLVARSKEALA

hIgG1 Fc

(SEQ ID NO: 15)
KSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQ
DWLNGKEYKCKVSNKALPAPIEKTISKAKGQPREPQVYTLPPSRDEL
TKNQVSLTCLVKGFYPSDIAVEWESNGQPENNYKTTPPVLDSDGSFF
LYSKLTVDKSRWQQGNVFSCSVMHEALHNHYTQKSLSLSPGK

### Primers

(1)
(SEQ ID NO: 16)
5'gggcggaccgatgctcataaatctgaagtcgc3' (F)
(2)
(SEQ ID NO: 17)
5'gggtttaaactctagatcatcaatgatgatgatgatggtgcaaac
caagtgcggcctgactggccgc3' (R)
(3)
(SEQ ID NO: 18)
5'cagactagatctgtgagcgatgtgccgcgtgatc3' (F)
(4)
(SEQ ID NO: 19)
5'cagactggatccgccaccgccgctgccaccaccgccagaaccgcc
accaccggtgcgatagttaatgctgatcgg3' (R)
(5)
(SEQ ID NO: 20)
5'cagactggatccgccaccgccgctgccaccaccgccagaaccgcc
accaccggtgcgatagttaatgctaatcggtttg3' (R)
(6)
(SEQ ID NO: 21)
5'cagactcatatggtgagcgatgtgccgcgtgatc3' (F)
(7)
(SEQ ID NO: 22)
5'ctgactggatccttaatggtgatgatgatgatgtgccgcagcaca
agctgcagcggtgcgatagttaatgctgatc3' (R)
(8)
(SEQ ID NO: 23)
5'ctgactggatccttaatggtgatgatgatgatgtgccgcagcaca
agctgcagcggtgcgatagttaatgctaatc3' (R)
(9)
(SEQ ID NO: 24)
5'cagactggatccgtgagcgatgtgccgcgtgatc3' (F)
(10)
(SEQ ID NO: 25)
5'ctgactaagctttcattaatggtgatgatgatgatgtgccgcagc
acaagctgcagcggtgcgatagttaatgctgatc3' (R)
(11)
(SEQ ID NO: 26)
5'ctgactaagctttcattaatggtgatgatgatgatgtgccgcagc
acaagctgcagcggtgcgatagttaatgctaatc3' (R)
(12)
(SEQ ID NO: 27)
5'cagactcatatggtgagcgatgtgccgcgtgatc3' (F)
(13)
(SEQ ID NO: 28)
5'ctgactggatccttaatggtgatgatgatgatgtgccgcagccta
agctgcagcggtgcgatagttaatgctgatc3' (R)
(14)
(SEQ ID NO: 29)
5'ctgactggatccttaatggtgatgatgatgatgtgccgcagccta
agctgcagcggtgcgatagttaatgctaatc3' (R)
(15)
(SEQ ID NO: 30)
5'cagactggatccgtgagcgatgtgccgcgtgatc3' (F)
(16)
(SEQ ID NO: 31)
5'ctgactaagctttcattaatggtgatgatgatgatgtgccgcagc
ctaagctgcagcggtgcgatagttaatgctgatc3' (R)
(17)
(SEQ ID NO: 32)
5'ctgactaagctttcattaatggtgatgatgatgatgtgccgcagc
ctaagctgcagcggtgcgatagttaatgctaatc3' (R)
(18)
(SEQ ID NO: 33)
5'gggcggaccggcaaatcttgtgacaaaactcacacatgc3' (F)
(19)
(SEQ ID NO: 34)
5'gggtttaaactctagatcatcaatgatgatgatgatggtgtttac
ccggagacagggagaggc3' (R)
(20)
(SEQ ID NO: 80)
5'cgtgcgagccagagcattagctcttacctgaactggtatcagcag
aaaccg3' (F)
(21)
(SEQ ID NO: 81)
5'cggtttctgctgataccagttcaggtaagagctaatgctctggct
cgcacg3' (R)
(22)
(SEQ ID NO: 82)
5'cgaaactgctgatttatcgcaacagcccgctgcagagcggtgtgc
c3' (F)
(23)
(SEQ ID NO: 83)
5'ggcacaccgctctgcagcgggctgttgcgataaatcagcagtttc
g3' (R)
(24)
(SEQ ID NO:84)
5'cctattattgccagcagacttaccgtgttccgccgacctttggcc
agggcacc3' (F)
(25)
(SEQ ID NO: 85)
5'ggtgccctggccaaaggtcggcggaacacggtaagtctgctggca
ataatagg3' (R)
(26)
(SEQ ID NO: 86)
5'gggcggaccgaagcacacaagagtgagatcgc3' (F)
(27)
(SEQ ID NO: 87)
5'gggtttaaacgggccctctagatcatcaatgatgatgatgatggt
gggctaaggcttctttgcttctagc 3' (R)
(28)
(SEQ ID NO: 88)
5'atggattccaaaacgccgttctggttcgatacacc 3' (F)
(29)
(SEQ ID NO: 89)
5'ggtgtatcgaaccagaacggcgttttggaatccat 3' (R)
(30)
(SEQ ID NO: 90)
5'accaaattggcaacagacgtcaccaaaatcaacaagg 3' (F)
(31)
(SEQ ID NO: 91)
5'ccttgttgattttggtgacgtctgttgccaatttggt 3' (R)

### EXAMPLES

### Example 1

### CDR Grafting

Using computational modeling, the CDR loop 1 (SGFTFSDYWM—SEQ ID NO: 35) and loop 3 (RSPSGFNR—SEQ ID NO: 36) from a TNF-binding nanobody (SEQ ID NO: 10) were grafted onto the framework of the wildtype tenth domain of the human fibronectin type III module (“¹⁰Fn3” or “wildtype Fn3”). The amino acid sequences of the TNF-binding nanobody and wildtype Fn3 molecule are as follows:

### TNF-Binding Nanobody (SEQ ID NO: 10)

QVQLVESGGGLVQPGGSLRLSCAASGFTFSDYWMYWVRQAPGKGLEW
VSEINTNGLITKYPDSVKGRFTISRDNAKNTLYLQMNSLKPEDTALY
YCARSPSGFNRGQGTQVTVSS

### Wildtype Fn3 (SEQ ID NO: 1)

VSDVPRDLEVVAATPTSLLISWDAPAVTVRYYRITYGETGGNSPVQE
FTVPGSKSTATISGLKPGVDYTITVYAVTGRGDSPASSKPISINYRT

Using the same methods, the CDR loop 1 (SQAIDSY—SEQ ID NO: 38) and loop 3 (QVVWRPFT—SEQ ID NO: 39) from a TNF-binding single domain antibody (SEQ ID NO: 40) were grafted onto wildtype Fn3. The amino acid sequence of the TNF-binding single domain antibody is as follows:

### TNF-Binding Single Domain Antibody (SEQ ID NO: 40)

Asp Ile Gln Met Thr Gln Ser Pro Ser Ser Leu Ser
Ala Ser Val Gly Asp Arg Val Thr Ile Thr Cys Arg
Ala Ser Gln Ala Ile Asp Ser Tyr Leu His Trp Tyr
Gln Gln Lys Pro Gly Lys Ala Pro Lys Leu Leu Ile
Tyr Ser Ala Ser Asn Leu Glu Thr Gly Val Pro Ser
Arg Phe Ser Gly Ser Gly Ser Gly Thr Asp Phe Thr
Leu Thr Ile Ser Ser Leu Leu Pro Glu Asp Phe Ala
Thr Tyr Tyr Cys Gln Gln Val Val Trp Arg Pro Phe
Thr Phe Gly Gln Gly Thr Lys Val Glu Ile Lys Arg

The DNA sequences for the formats shown below were then optimised for expression in E. coli and prepared at Geneart AG, Germany. The resulting DNA fragments were digested with NdeI/BamHI and ligated into the corresponding sites of pET9a (appropriate flanking DNA sequences were added to the formats below).

### Formats:

1) wildtype Fn3 with CDR1 and CDR3 loops from TNF binding nanobody-His tag (pET9a)

(SEQ ID NO: 41)
VSDVPRDLEVVAATPTSLLISWDASGFTFSDYWMRITYGETGGNSPV
QEFTVPGSKSTATISGLKPGVDYTITVYRSPSGFNRISINYRTHHHH
HH

2) wildtype Fn3 with CDR1 and CDR3 loops from TNF binding nanobody-His tag (pET9a) in which the first 8 amino acids are removed from the sequence.

(SEQ ID NO: 42)
EVVAATPTSLLISWDASGFTFSDYWMRITYGETGGNSPVQEFTVPGS
KSTATISGLKPGVDYTITVYRSPSGFNRISINYRTHHHHHH

3) wildtype Fn3 with CDR1 and CDR3 loops from TNF binding single domain antibody-His tag (pET9a)

(SEQ ID NO: 43)
VSDVPRDLEVVAATPTSLLISWDASQAIDSYYRITYGETGGNSPVQE
FTVPGSKSTATISGLKPGVDYTITVYQVVWRPFTPISINYRTHHHHH
H

4) wildtype Fn3 with CDR1 and CDR3 loops from TNF binding single domain antibody-His tag (pET9a) in which the first 8 amino acids are removed from the sequence

(SEQ ID NO: 44)
EVVAATPTSLLISWDASQAIDSYYRITYGETGGNSPVQEFTVPGSKS
TATISGLKPGVDYTITVYQVVWRPFTPISINYRTHHHHHH

The ligation mix was used to transform XL1-Blue or DH5alpha competent cells. Positive clones were verified by DNA sequencing. Constructs were expressed in several E. coli strains including BL21 (DE3). After induction and expression, cell pellets were frozen at −20° C. and then resuspended in lysis buffer (20 mM NaH₂PO₄, 10 mM Imidazol, 500 mM NaCl, 1 tablet Complete without EDTA per 50 ml buffer (Roche), 2 mM MgCl₂, 10 U/ml Benzonase (Merck) [pH7.4]. Cells were sonicated on ice and centrifuged. Supernatant was filtered and loaded onto a Ni-NTA column. Column was washed with Wash buffer (as for lysis buffer but with 20 mM Imidazol) and then eluted with Elution Buffer (as for lysis buffer but up to 500 mM Imidazol). Samples were analysed on Bis-Tris Gels (Invitrogen), then concentrated in Amicon Ultra-15 tubes, loaded onto a Superdex prep grade column (Amersham) and eluted with 10 mM Tris or PBS. Samples were analysed again on Bis-Tris gels.

### Example 2

### Identification of Positions within the Fibronectin Molecule for Amino Acid Modifications

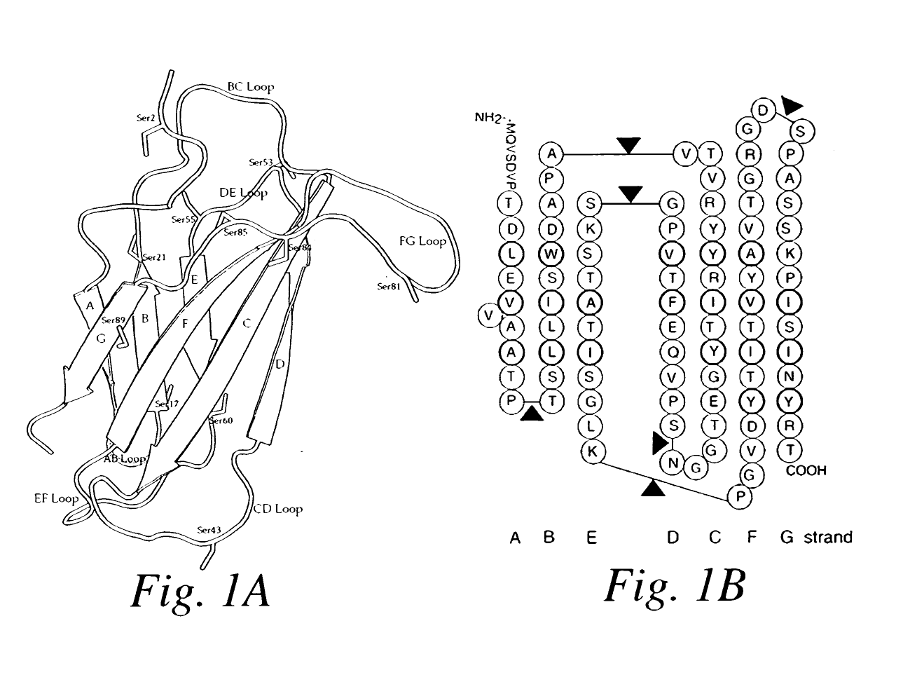

Based on a review of the wildtype Fn3 sequence, positions were identified as potential sites for amino acid modifications, e.g., for substitution with cysteine or non-naturally occurring amino acid residues to facilitate PEGylation. For example, the serine residues were analyzed as set forth below. There are 11 total Ser residues which are underlined in the sequence below; see also FIG. 1 which shows the wildtype Fn3 molecule with a stick representation of the serine residues)

### Wildtype Fn3

(SEQ ID NO: 1)
VSDVPRDLEVVAATPTSLLISWDAPAVTVRYYRITYGETGGNSPVQE
FTVPGSKSTATISGLKPGVDYTITVYAVTGRGDSPASSKPISINYRT

Serine residues which are located near the binding surface were excluded from the analysis, e.g., Ser 2 which belongs to the N-terminal region and which also contacts with the FG and BC loops (Ser residue underlined in the sequence below).

(SEQ ID NO: 1)
VSDVPRDLEVVAATPTSLLISWDAPAVTVRYYRITYGETGGNSPVQEF
TVPGSKSTATISGLKPGVDYTITVYAVTGRGDSPASSKPISINYRT

Ser 53-Ser 55—These residues belong to the DE loop (underlined below).

(SEQ ID NO: 1)
VSDVPRDLEVVAATPTSLLISWDAPAVTVRYYRITYGETGGNSPVQEF
TVPGSKSTATISGLKPGVDYTITVYAVTGRGDSPASSKPISINYRT

Ser 81-Ser 84-Ser 85—These residues belong to the FG loop (underlined below).

(SEQ ID NO: 1)
VSDVPRDLEVVAATPTSLLISWDAPAVTVRYYRITYGETGGNSPVQEF
TVPGSKSTATISGLKPGVDYTITVYAVTGRGDSPASSKPISINYRT

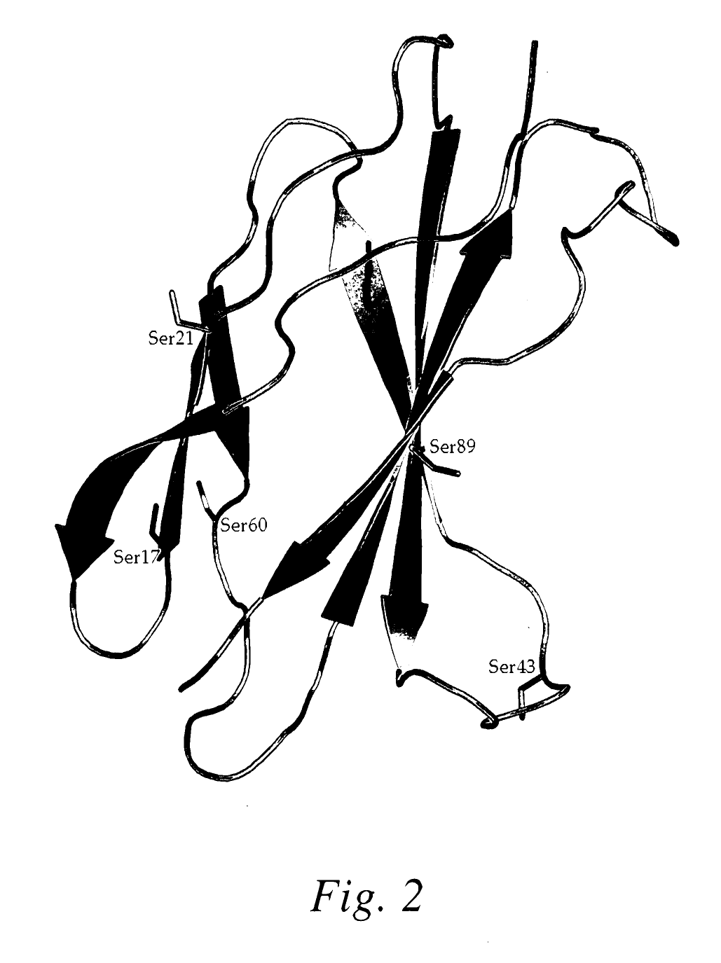

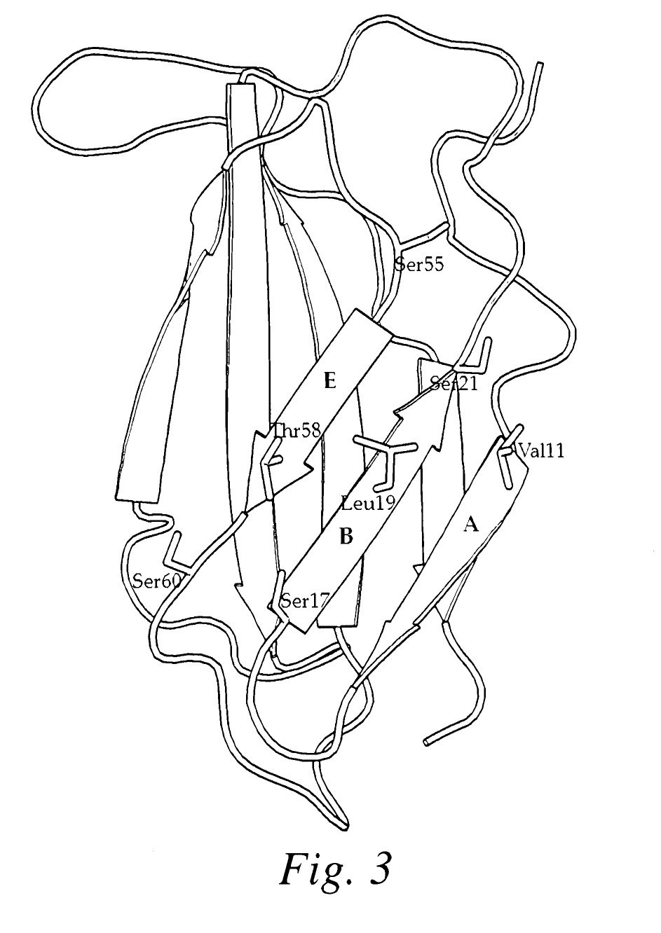

The Serine candidates for modifications include: Ser 17-Ser 21-Ser 43-Ser 60-Ser 89. These Serine residues are all exposed to solvent and they are all part of a beta-strand except Ser 43. (see FIG. 2).
Ser 17 and Ser 21 are located at the beginning and end of the B strand, respectively. Ser 60 is positioned at the end of the E strand.
Ser 21 and Ser 60 are located on the two adjacent strands which form the three-stranded sheet of fibronectin.
Ser 89 is positioned in the middle of the G strand, which is also the last strand forming the 4-stranded sheet. Accordingly, Ser 89 is also exposed to solvent and accessible to external molecules.
Ser 43 is located at the bottom of the molecule and belongs to the CD loop, at the end of the loop that is bent towards the solvent (see FIG. 2).

Other residues for potential modification sites include the following residues which are located on beta strands and exposed to solvent: V11-L19-T58-T71 (Underlined in the sequence below)

(SEQ ID NO: 1)
VSDVPRDLEVVAATPTSLLISWDAPAVTVRYYRITYGETGGNSPVQEF
TVPGSKSTATISGLKPGVDYTITVYAVTGRGDSPASSKPISINYRT

With reference to FIG. 3, the three-stranded sheet is shown (strands A-B-E). At the bottom of the sheet there are located the candidate residues Ser 17 and Ser 60. The candidate residue, Ser 21, is located at the top. Ser 55 has been excluded because it is close to the binding surface.

Val 11 which is located close to the start of strand A appears not to be conserved in the fibronectin module sequences.

Leu 19 which is located in the middle of strand B also is not a conserved position.

Thr 58 is located at the end of strand E.

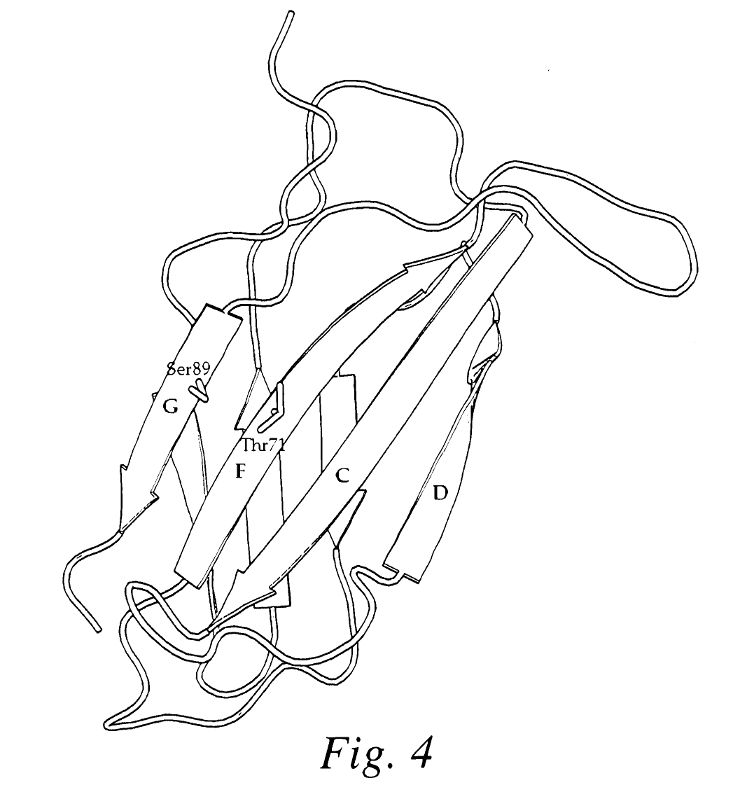

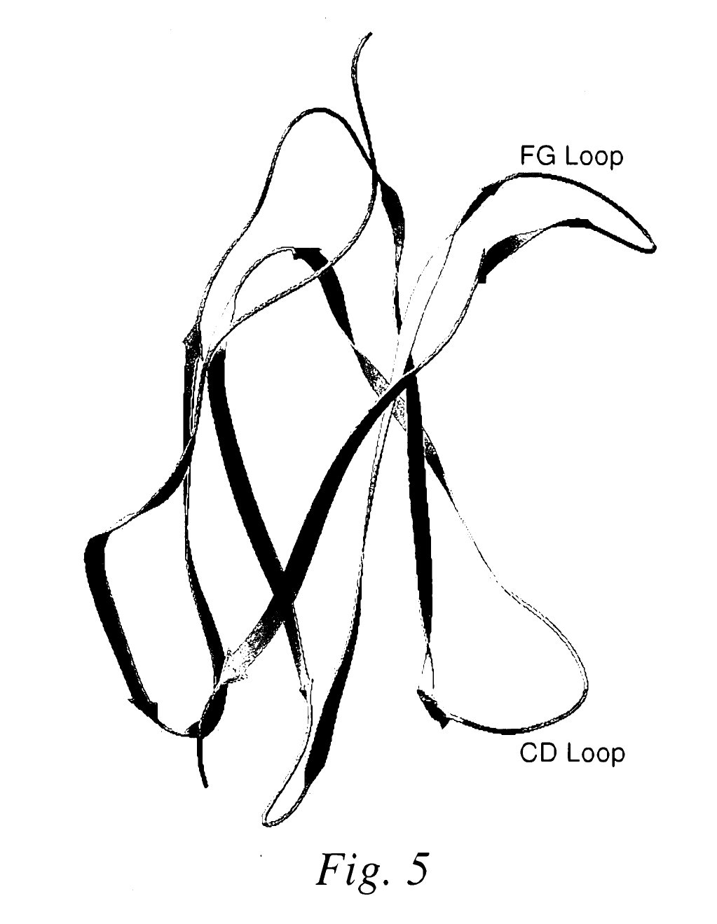

With reference to FIG. 4 (the other side of the scaffold; 4-stranded sheet), Thr 71 is located close to Ser 89. This position is also not conserved. To be noticed is that this part of the fibronectin molecule forms a kind of “C” structure. The FG loop and the CD loop are looking towards each other (see FIG. 5).

Depending on the size of PEG molecules to attach to the molecule, this side of the molecule may not be amenable to PEGylation.

### Example 3

### PEGylation of Fn3 Sequences

To increase the half-life of Fn, PEGylation of TNF-binding Fn3 (SEQ ID NO:3), TNF-binding Fn3 (R18L and I56T) (SEQ ID NO:4), wildtype Fn3 (SEQ ID NO:1) and wildtype Fn3 (RGD to RGA) (SEQ ID NO: 2) using (1) cysteine and (2) non-natural amino acids was conducted as follows.

### TNF-Binding Fn3

(SEQ ID NO: 3)
VSDVPRDLEVVAATPTSRLISWNRSGLQSRYYRITYGETGGNSPVQEF
TVPPWASIATISGLKPGVDYTITVYAVTDKSDTYKYDDPISINYRT

### TNF-Binding Fn3 (R18L and I56T)

(SEQ ID NO: 4)
VSDVPRDLEVVAATPTSLLISWNRSGLQSRYYRITYGETGGNSPVQEF
TVPPWASTATISGLKPGVDYTITVYAVTDKSDTYKYDDPISINYRT

### Wildtype Fn3

(SEQ ID NO: 1)
VSDVPRDLEVVAATPTSLLISWDAPAVTVRYYRITYGETGGNSPVQEF
TVPGSKSTATISGLKPGVDYTITVYAVTGRGDSPASSKPISINYRT

### Wildtype Fn3 Sequence (RGD to RGA)

(SEQ ID NO: 2)
VSDVPRDLEVVAATPTSLLISWDAPAVTVRYYRITYGETGGNSPVQEF
TVPGSKSTATISGLKPGVDYTITVYAVTGRGASPASSKPISINYRT

PEGylation Using Cysteine

The DNA sequences corresponding to the foregoing TNF-binding Fn3 and wildtype Fn3 sequences were optimised for expression in E. coli and prepared at Geneart AG, Germany. For insertion of a C-terminal cysteine residue, the TNF-binding sequences were amplified using primers 6 (SEQ ID NO:21) and 7 (SEQ ID NO:22), and the wild-type sequences were amplified using primers 6 (SEQ ID NO:21) and 8 (SEQ ID NO:23) (see primers described above in Materials and Methods section). PCR products were digested with NdeI/BamHI and cloned into the corresponding sites of pET9a. In addition, the TNF-binding sequences were amplified using primers 9 (SEQ ID NO: 24) and 10 (SEQ ID NO: 25) and the wild-type sequences were amplified using primers 9 (SEQ ID NO: 24) and 11 (SEQ ID NO: 26). PCR products were digested with BamHI/HindIII and cloned into the corresponding sites of pQE-80L with dsbA signal sequence.

### Formats:

1) TNF-binding Fn3 sequence-3xA linker-C-3xA linker-His tag (pET9a)

(SEQ ID NO: 48)
VSDVPRDLEVVAATPTSRLISWNRSGLQSRYYRITYGETGGNSPVQEF
TVPPWASIATISGLKPGVDYTITVYAVTDKSDTYKYDDPISINYRTAA
ACAAAHHHHHH

2) TNF-binding Fn3 (R18L and I56T) sequence-3xA linker-C-3xA linker-His tag (pET9a)

(SEQ ID NO: 49)
VSDVPRDLEVVAATPTSLLISWNRSGLQSRYYRITYGETGGNSPVQEF
TVPPWASTATISGLKPGVDYTITVYAVTDKSDTYKYDDPISINYRTAA
ACAAAHHHHHH

3) wildtype Fn3 sequence-3xA linker-C-3xA linker-His tag (pET9a)

(SEQ ID NO: 50)
VSDVPRDLEVVAATPTSLLISWDAPAVTVRYYRITYGETGGNSPVQEF
TVPGSKSTATISGLKPGVDYTITVYAVTGRGDSPASSKPISINYRTAA
ACAAAHHHHHH

4) wildtype Fn3 (RGD to RGA) sequence-3xA linker-C-3xA linker-His tag (pET9a)

(SEQ ID NO: 51)
VSDVPRDLEVVAATPTSLLISWDAPAVTVRYYRITYGETGGNSPVQEF
TVPGSKSTATISGLKPGVDYTITVYAVTGRGASPASSKPISINYRTAA
ACAAAHHHHHH

4) dsbA signal sequence-TNF-binding Fn3 sequence-3xA linker-C-3xA linker-His tag (pQE-80L)

(SEQ ID NO: 52)
MKKIWLALAGLVLAFSASAGSVSDVPRDLEVVAATPTSRLISWNRSGL
QSRYYRITYGETGGNSPVQEFTVPPWASIATISGLKPGVDYTITVYAV
TDKSDTYKYDDPISINYRTAAACAAAHHHHHH

5) dsbA signal sequence-TNF-binding Fn3 (R18L and I56T) sequence-3xA linker-C-3xA linker-His tag (pQE-80L)

(SEQ ID NO: 53)
MKKIWLALAGLVLAFSASAGSVSDVPRDLEVVAATPTSLLISWNRSGL
QSRYYRITYGETGGNSPVQEFTVPPWASTATISGLKPGVDYTITVYAV
TDKSDTYKYDDPISINYRTAAACAAAHHHHHH

6) dsbA signal sequence-wildtype Fn3 sequence-3xA linker-C-3xA linker-His tag (pQE-80L)

(SEQ ID NO: 54)
MKKIWLALAGLVLAFSASAGSVSDVPRDLEVVAATPTSLLISWDAPAV
TVRYYRITYGETGGNSPVQEFTVPGSKSTATISGLKPGVDYTITVYAV
TGRGDSPASSKPISINYRTAAACAAAHHHHHH

7) dsbA signal sequence-wildtype Fn3 (RGD to RGA) sequence-3xA linker-C-3xA linker-His tag (pQE-80L)

(SEQ ID NO: 55)
MKKIWLALAGLVLAFSASAGSVSDVPRDLEVVAATPTSLLISWDAPAVTV
RYYRITYGETGGNSPVQEFTVPGSKSTATISGLKPGVDYTITVYAVTGRG
ASPASSKPISINYRTAAACAAAHHHHHH

8) wildtype Fn3 sequence-(RGD to RGA) His tag (pET9a)

(SEQ ID NO: 37)
MVSDVPRDLEVVAATPTSLLISWDAPAVTVRYYRITYGETGGNSPVQEFT
VPGSKSTATISGLKPGVDYTITVYAVTGRGASPASSKPISINYRTHHHHH
H

The ligation mix was used to transform XL1-Blue or DH5alpha competent cells. Positive clones were verified by DNA sequencing. Constructs were expressed in several E. coli strains including KS474, TG1 (−) and BL21 (DE3). After induction and expression, cell pellets were frozen at −20° C. and then resuspended in lysis buffer (20 mM NaH₂PO₄, 10 mM Imidazol, 500 mM NaCl, 1 tablet Complete without EDTA per 50 ml buffer (Roche), 2 mM MgCl₂, 10 U/ml Benzonase (Merck) [pH7.4]. Cells were sonicated on ice and centrifuged. Supernatant was filtered and loaded onto a Ni-NTA column. The column was washed with Wash buffer (as for lysis buffer but with 20 mM Imidazol) and then eluted with Elution Buffer (as for lysis buffer but up to 500 mM Imidazol). Samples were analysed on Bis-Tris Gels (Invitrogen), then concentrated in Amicon Ultra-15 tubes, loaded onto a Superdex prep grade column (Amersham) and eluted with PBS [pH6.5 to 7.2] (a mild reduction was sometimes used before gel filtration). Samples were analysed again on Bis-Tris gels. Purified protein was supplemented with DTT (final concentration of 10 μM) and then filtered through an Amicon Ultra-4 tube, 100k to remove endotoxin. A HiTrap Desalting Column was used for DTT removal. Sample in 50 mM MES buffer at a pH of 5.5, was coupled for approximately 4 hours at room temperature with 5 to 10 molar excess PEG-maleimide, efficiency of PEGylation was analysed by SDS-PAGE and MS. Excess PEG was removed via a HiTrap-SP-FF column followed by dialysis with PBS or Tris. Binding to corresponding antigen was verified by ELISA. The site of PEGylation was determined by reduction, alkylation and trypsin digest. 100 μg of sample was dried and incubated in a final volume of 100 μl with 6.4M urea, 0.32M NH₄CO₃ and 0.01M DTT for 30 min at 50° C. under Argon, IAA was then added (0.03M) and incubated for 15 min at room temp in the dark. The sample was desalted, dried, and then incubated in a final volume of 50 μl with 0.8M urea, 0.04M NH₄CO₃, 0.02M Tris, pH10 and 1 μg trypsin and analysed by LC-MS.

The half-life of these constructs was determined in vivo. 10 mg/kg of each compound was administered intravenously into Lewis rats (n=3), samples were taken at pre-dose, 1 2, 4, 8, 24, 48, 96, 192 and 384 hrs. Biacore analysis was performed using a CM5 chip with standard amine coupling. Flow cell 1 was blank (surface activation with EDC/NHS and subsequent deactivation with Ethanolamine) for reference subtraction. Flow cell 2 was coated with THE anti-HIS mAb (GenScript Corp) for PK read-out. Flow cells 3 and 4 were coated with compounds that were administered to the animals (surface saturation) for immunogenicity read-out. Rat serum samples were diluted 1:8 with HBS-EP and NBSreducer (Biacore; final conc. 1 mg/ml). A standard curve was prepared for compound quantification, a 1:2 dilution series from 20 mg/l down to 0.078 mg/l of the corresponding compound that was administered to the animals was prepared in rat serum (GeneTex). The rat serum was diluted 1:8 with HBS-EP and 1 mg/ml NSBreducer. The standard curve data were fitted using XLfit 4.2 and used to calculate the compound concentrations in the serum samples (PK). The compound half-life was calculated using the WinNonlin software. PK data were fitted using a non-compartmental model.

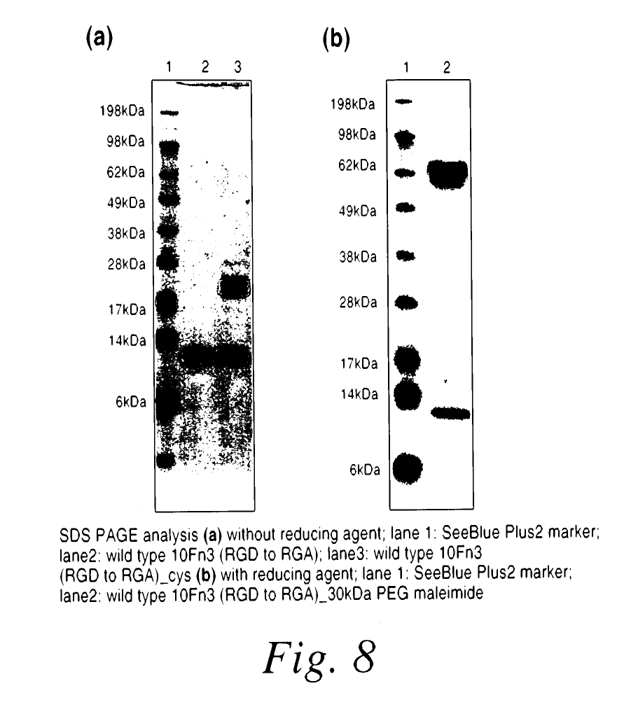

Wild type 10Fn3 (RGD to RGA) and wild type 10Fn3 (RGD to RGA)_cys were expressed in E. coli, purified and analysed by SDS PAGE (FIG. 8a). In addition to monomers, dimers were also observed for the cysteine variant. LC-MS showed a mass of 10.85 kDa for unmodified and 11.38 kDa for the cysteine variant, these molecular weights corresponded to the expected proteins (data not shown).

Wild type 10Fn3 (RGD to RGA)_cys was modified with 30 kDa PEG-maleimide. FIG. 8b showed presence of PEGylated protein by SDS-PAGE, this was further confirmed by MALDI-TOF_MS. The PEGylated sample showed a MW of 42.8 kDa, a broad peak was due to the PEG. The site of PEGylation was determined by LC-MS analytics of reduced, alkylated and trypsin digested PEGylated and non-PEGylated samples (date not shown). Comparison of the peptide maps showed that the peak at RT 10.89 min was missing in the PEGylated sample. This peptide had a monoisotropic MW of 1527.7 Da corresponding to T[95-108]H (peptide containing cysteine at position 99) of the expected protein (data not shown).

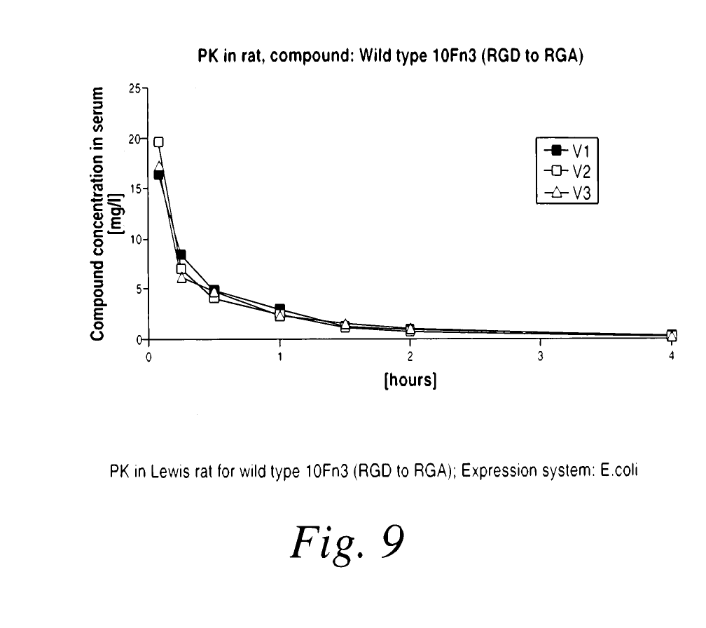

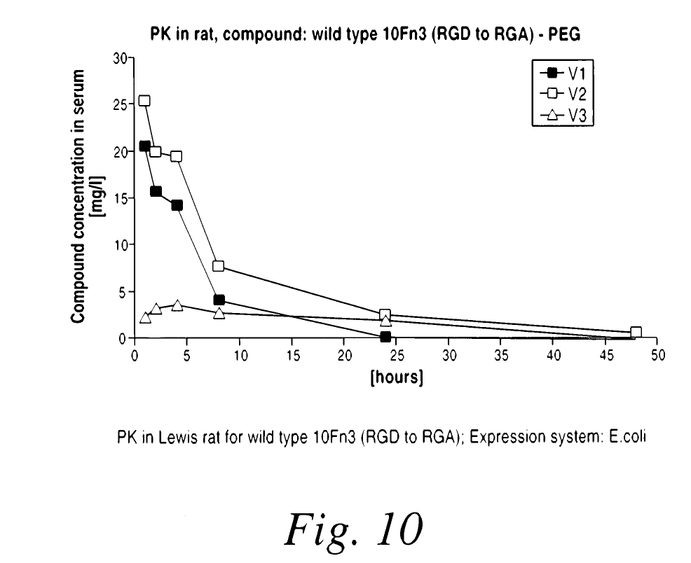

In vivo data showed a significant half-life improvement for PEGylated wild type 10Fn3 (FIG. 10) when compared with unmodified 10Fn3 (FIG. 9). The average half-life for unmodified 10Fn3 was 0.52 h, this increased to 3.6 h for PEGylated 10Fn3 (FIG. 11). No signals could be detected with animal EV3.

The results of this rat study demonstrate that the in vivo serum half-life of Fibronectin (10Fn3) can be significantly extended when prepared as a PEGylated conjugate.

To extrapolate in vivo half-life results from the rat study to humans, the following formula is used:

$${t}_{\frac{1}{2} human} \approx {( \frac{70 kg}{0.240 kg} )}^{0.25} {t}_{\frac{1}{2} rat} \approx 4.13 \times {t}_{\frac{1}{2} rat} \qquad Formula 1$$

where the exponent 0.25 is empirical and provides a good basis for extrapolation with species having similar clearance mechanisms. (See e.g., West et al. (1997) Science 276: 122-126; Bazin-Redureau et al. (1998) Toxicology and applied pharmacology 150: 295-300; and Dedrick (1973) J. Pharmacokinetics and Biopharmaceuticals 5: 435-461. Using Formula 1, the extrapolated average half-life in man is expected to be about 14.9 hours.

The average fold increase of half life with the conjugated Fn3 molecule can be calculated by dividing the average half-life of the conjugated Fn3 molecule by the average half-life of the unconjugated Fn3 molecule. For example, with average Fn3-PEG conjugate (3.6) divided by average unconjugated Fn3 (0.52), resulting in approximately 7 fold increase in half-life of the PEG-Fn3 conjugate in vivo.

PEGylation Using Non-Natural Amino Acids

The DNA sequences described above corresponding to the TNF-binding Fn3 (SEQ ID NO: 3 and SEQ ID NO: 4) and wildtype Fn3 (SEQ ID NO: 1 and SEQ ID NO: 2) sequences were optimised for expression in E. coli and prepared at Geneart AG, Germany. For insertion of a C-terminal amber codon, the TNF-binding sequences (SEQ ID NO: 3 and SEQ Id NO: 4) were amplified using primers 12 (SEQ ID NO: 27) and 13 (SEQ ID NO: 28) and the wild-type sequences (SEQ ID NO: 1 and SEQ ID NO: 2) were amplified using primers 12 (SEQ ID NO: 27) and 14 (SEQ ID NO: 29). PCR products were digested with NdeI/BamHI and cloned into the corresponding sites of pET9a. In addition, the TNF-binding sequences (SEQ ID NO: 3 and SEQ ID NO: 4) were also amplified using primers 15 (SEQ ID NO: 30) and 16 (SEQ ID NO: 31) and the wild-type sequences (SEQ ID NO: 1 and SEQ ID NO: 2) were amplified using primers 15 (SEQ ID NO: 30) and 17 (SEQ ID NO: 32). PCR products were digested with BamHI/HindIII and cloned into the corresponding sites of pQE-80L with dsbA signal sequence.

### Formats:

1) TNF-binding Fn3 sequence-3xA linker-amber codon-3xA linker-His tag (pET9a)

(SEQ ID NO: 56)
VSDVPRDLEVVAATPTSRLISWNRSGLQSRYYRITYGETGGNSPVQEFTV
PPWASIATISGLKPGVDYTITVYAVTDKSDTYKYDDPISINYRTAAA*AA
AHHHHHH

2) TNF-binding Fn3 (R18L and I56T) sequence-3xA linker-amber codon-3xA linker-His tag (pET9a)

(SEQ ID NO: 57)
VSDVPRDLEVVAATPTSLLISWNRSGLQSRYYRITYGETGGNSPVQEFTV
PPWASTATISGLKPGVDYTITVYAVTDKSDTYKYDDPISINYRTAAA*AA
AHHHHHH

3) wildtype Fn3 sequence-3xA linker-amber codon-3xA linker-His tag (pET9a)

(SEQ ID NO: 58)
VSDVPRDLEVVAATPTSLLISWDAPAVTVRYYRITYGETGGNSPVQEFTV
PGSKSTATISGLKPGVDYTITVYAVTGRGDSPASSKPISINYRTAAA*AA
AHHHHHH

4) wildtype Fn3 (RGD to RGA) sequence-3xA linker-amber codon-3xA linker-His tag (pET9a)

(SEQ ID NO: 59)
VSDVPRDLEVVAATPTSLLISWDAPAVTVRYYRITYGETGGNSPVQEFTV
PGSKSTATISGLKPGVDYTITVYAVTGRGASPASSKPISINYRTAAA*AA
AHHHHHH

5) dsbA signal sequence-TNF-binding Fn3 sequence-3xA linker-amber codon-3xA linker-His tag (pQE-80L)

(SEQ ID NO: 60)
MKKIWLALAGLVLAFSASAGSVSDVPRDLEVVAATPTSRLISWNRSGLQS
RYYRITYGETGGNSPVQEFTVPPWASIATISGLKPGVDYTITVYAVTDKS
DTYKYDDPISINYRTAAA*AAAHHHHHH

6) dsbA signal sequence-TNF-binding Fn3 (R18L and I56T) sequence-3xA linker-amber codon-3xA linker-His tag (pQE-80L)

(SEQ ID NO: 61)
MKKIWLALAGLVLAFSASAGSVSDVPRDLEVVAATPTSLLISWNRSGLQS
RYYRITYGETGGNSPVQEFTVPPWASTATISGLKPGVDYTITVYAVTDKS
DTYKYDDPISINYRTAAA*AAAHHHHHH

7) dsbA signal sequence-wildtype Fn3 sequence-3xA linker-amber codon-3xA linker-His tag (pQE-80L)

(SEQ ID NO: 62)
MKKIWLALAGLVLAFSASAGSVSDVPRDLEVVAATPTSLLISWDAPAVTV
RYYRITYGETGGNSPVQEFTVPGSKSTATISGLKPGVDYTITVYAVTGRG
DSPASSKPISINYRTAAA*AAAHHHHHH

8) dsbA signal sequence-wildtype Fn3 (RGD to RGA) sequence-3xA linker-amber codon-3xA linker-His tag (pQE-80L)

(SEQ ID NO: 63)
MKKIWLALAGLVLAFSASAGSVSDVPRDLEVVAATPTSLLISWDAPAVTV
RYYRITYGETGGNSPVQEFTVPGSKSTATISGLKPGVDYTITVYAVTGRG
ASPASSKPISINYRTAAA*AAAHHHHHH

*denotes position of non-natural amino acid

The ligation mix was used to transform XL1-Blue or DH5alpha competent cells. Positive clones were verified by DNA sequencing. Constructs above and pAmber-AcPheRS were co-transformed and expressed in several E. coli strains including KS474, TG1 (−), BL21 (DE3) and DH10B, media contained 1 mM p-acetyl-L-phenylalanine. After induction and expression, cell pellets were frozen at −20° C. and then resuspended in lysis buffer (20 mM NaH₂PO₄, 10 mM Imidazol, 500 mM NaCl, 1 tablet Complete without EDTA per 50 ml buffer (Roche), 2 mM MgCl₂, 10 U/ml Benzonase (Merck) [pH7.4]. Cells were sonicated on ice and centrifuged. Supernatant was filtered and loaded onto a Ni-NTA column. Column was washed with Wash buffer (as for lysis buffer but with 20 mM Imidazol) and then eluted with Elution Buffer (as for lysis buffer but up to 500 mM Imidazol). Samples were analysed on Bis-Tris Gels (Invitrogen), then concentrated in Amicon Ultra-15 tubes, loaded onto a Superdex prep grade column (Amersham) and eluted with 10 mM Tris. Samples were analysed again on Bis-Tris gels. Purified protein was dialysed against 100 mM sodium acetate, pH 5.5 and coupled with 5 to 10 molar excess PEG-hydrazide for approximately 2 hours at room temperature. Efficiency of PEGylation was analysed by SDS-PAGE and SEC. pH was then increased with concentrated Tris and excess PEG was removed by Ni-NTA chromatography followed by dialysis with PBS or Tris.

### Example 4

### Serum Albumin (HSA) Fusion of Fn3 Sequences

Fibronectin—serum albumin fusion molecules were made using the TNF-binding Fn3 sequence (SEQ ID NO: 3), TNF-binding Fn3 (R18L and I56T) (SEQ ID NO: 4), wildtype Fn3 sequence (SEQ ID NO: 1), wildtype Fn3 (RGD to RGA) (SEQ ID NO: 2) or VEGFR-binding FN3 (SEQ ID NO: 76) described above combined with anti-HSA (SEQ ID NO: 12), anti-MSA (SEQ ID NO: 13), anti-RSA binder molecules (SEQ ID NO: 78), RSA (SEQ ID NO: 79), or HSA (SEQ ID NO: 14).

(i) Anti-HSA, Anti-MSA or Anti-RSA Fusion Molecules

The DNA sequence for the anti-HSA binder (SEQ ID NO: 12) or the anti-MSA binder (SEQ ID NO: 13) were optimised for expression in E. coli and prepared at Geneart AG, Germany. The resulting DNA fragment was ligated into pQE-80L with dsbA signal sequence using BamHI/HindIII (appropriate flanking DNA sequences were added). The DNA sequences corresponding to the TNF-binding Fn3 sequences (SEQ ID NO: 3 and SEQ ID NO: 4) and wildtype Fn3 sequences (SEQ ID NO: 1 and SEQ ID NO: 2) were optimised for expression in E. coli and prepared at Geneart AG, Germany. The resulting DNA fragments were amplified using primers 3 (SEQ ID NO: 18) and 4 (SEQ ID NO: 19) for TNF-binding Fn3 sequences (SEQ ID NO: SEQ ID NO: 3 and SEQ ID NO: 4) or primers 3 (SEQ ID NO: 18) and 5 (SEQ ID NO: 20) for the wildtype Fn3 sequences (SEQ ID NO: 1 and SEQ ID NO: 2), digested with BglII/BamHI and ligated into the BamHI site of pQE-80L-dsbA-antiHSA or pQE-80L-dsbA-antiMSA. Wild type Fn3 (RGD to RGA)-GS linker-anti-RSA His (SEQ ID NO: 92) was prepared from wildtype Fn3 (RGD to RGA)-GS linker-anti-MSA His (SEQ ID NO: 71) in pQE-80L by site directed mutagenesis. The first mutagenesis, IKHLK to SSYLN, was performed with primers 20 (SEQ ID NO: 80) and 21 (SEQ ID NO: 81); the second mutagenesis, GASR to RNSP, was performed with primers 22 (SEQ ID NO: 82) and 23 (SEQ ID NO: 83); and the third mutagenesis, GARWPQ to TYRVPP, was performed with primers 24 (SEQ ID NO: 84) and 25 (SEQ ID NO: 85).

### Formats:

1) dsbA signal sequence-TNF-binding Fn3 sequence-GS linker-anti-HSA-His tag (pQE-80L)

(SEQ ID NO: 64)
MKKIWLALAGLVLAFSASAGSVSDVPRDLEVVAATPTSRLISWNRSGLQS
RYYRITYGETGGNSPVQEFTVPPWASIATISGLKPGVDYTITVYAVTDKS
DTYKYDDPISINYRTGGGGSGGGGSGGGGSEVQLLESGGGLVQPGGSLRL
SCAASGFTFDEYNMSWVRQAPGKGLEWVSTILPHGDRTYYADSVKGRFTI
SRDNSKNTLYLQMNSLRAEDTAVYYCAKQDPLYRFDYWGQGTLVTVSSHH
HHHH

2) dsbA signal sequence-TNF-binding Fn3 (R18L and I56T) sequence-GS linker-anti-HSA-His tag (pQE-80L)

(SEQ ID NO: 65)
MKKIWLALAGLVLAFSASAGSVSDVPRDLEVVAATPTSLLISWNRSGLQS
RYYRITYGETGGNSPVQEFTVPPWASTATISGLKPGVDYTITVYAVTDKS
DTYKYDDPISINYRTGGGGSGGGGSGGGGSEVQLLESGGGLVQPGGSLRL
SCAASGFTFDEYNMSWVRQAPGKGLEWVSTILPHGDRTYYADSVKGRFTI
SRDNSKNTLYLQMNSLRAEDTAVYYCAKQDPLYRFDYWGQGTLVTVSSHH
HHHH

3) dsbA signal sequence-wildtype Fn3 sequence-GS linker-anti HSA-His tag (pQE-80L)

(SEQ ID NO: 66)
MKKIWLALAGLVLAFSASAGSVSDVPRDLEVVAATPTSLLISWDAPAVTV
RYYRITYGETGGNSPVQEFTVPGSKSTATISGLKPGVDYTITVYAVTGRG
DSPASSKPISINYRTGGGGSGGGGSGGGGSEVQLLESGGGLVQPGGSLRL
SCAASGFTFDEYNMSWVRQAPGKGLEWVSTILPHGDRTYYADSVKGRFTI
SRDNSKNTLYLQMNSLRAEDTAVYYCAKQDPLYRFDYWGQGTLVTVSSHH
HHHH

4) dsbA signal sequence-wildtype Fn3 (RGD to RGA) sequence-GS linker-anti HSA-His tag (pQE-80L)

(SEQ ID NO: 67)
MKKIWLALAGLVLAFSASAGSVSDVPRDLEVVAATPTSLLISWDAPAVTV
RYYRITYGETGGNSPVQEFTVPGSKSTATISGLKPGVDYTITVYAVTGRG
ASPASSKPISINYRTGGGGSGGGGSGGGGSEVQLLESGGGLVQPGGSLRL
SCAASGFTFDEYNMSWVRQAPGKGLEWVSTILPHGDRTYYADSVKGRFTI
SRDNSKNTLYLQMNSLRAEDTAVYYCAKQDPLYRFDYWGQGTLVTVSSHH
HHHH

5) dsbA signal sequence-TNF-binding Fn3 sequence-GS linker-anti-MSA-His tag (pQE-80L)

(SEQ ID NO: 68)
MKKIWLALAGLVLAFSASAGSVSDVPRDLEVVAATPTSRLISWNRSGLQS
RYYRITYGETGGNSPVQEFTVPPWASIATISGLKPGVDYTITVYAVTDKS
DTYKYDDPISINYRTGGGGSGGGGSGGGGSDIQMTQSPSSLSASVGDRVT
ITCRASQSIIKHLKWYQQKPGKAPKLLIYGASRLQSGVPSRFSGSGSGTD
FTLTISSLQPEDFATYYCQQGARWPQTFGQGTKVEIKRHHHHHH

6) dsbA signal sequence-TNF-binding Fn3 (R18L and I56T) sequence-GS linker-anti-MSA-His tag (pQE-80L)

(SEQ ID NO: 69)
MKKIWLALAGLVLAFSASAGSVSDVPRDLEVVAATPTSLLISWNRSGL
QSRYYRITYGETGGNSPVQEFTVPPWASTATISGLKPGVDYTITVYAV
TDKSDTYKYDDPISINYRTGGGGSGGGGSGGGGSDIQMTQSPSSLSAS
VGDRVTITCRASQSIIKHLKWYQQKPGKAPKLLIYGASRLQSGVPSRF
SGSGSGTDFTLTISSLQPEDFATYYCQQGARWPQTFGQGTKVEIKRHH
HHHH

7) dsbA signal sequence-wildtype Fn3 sequence-GS linker-anti-MSA-His tag (pQE-80L)

(SEQ ID NO: 70)
MKKIWLALAGLVLAFSASAGSVSDVPRDLEVVAATPTSLLISWDAPAV
TVRYYRITYGETGGNSPVQEFTVPGSKSTATISGLKPGVDYTITVYAV
TGRGDSPASSKPISINYRTGGGGSGGGGSGGGGSDIQMTQSPSSLSAS
VGDRVTITCRASQSIIKHLKWYQQKPGKAPKLLIYGASRLQSGVPSRF
SGSGSGTDFTLTISSLQPEDFATYYCQQGARWPQTFGQGTKVEIKRHH
HHHH

8) dsbA signal sequence-wildtype Fn3 (RGD to RGA) sequence-GS linker-anti-MSA-His tag (pQE-80L)

(SEQ ID NO: 71)
MKKIWLALAGLVLAFSASAGSVSDVPRDLEVVAATPTSLLISWDAPAV
TVRYYRITYGETGGNSPVQEFTVPGSKSTATISGLKPGVDYTITVYAV
TGRGASPASSKPISINYRTGGGGSGGGGSGGGGSDIQMTQSPSSLSAS
VGDRVTITCRASQSIIKHLKWYQQKPGKAPKLLIYGASRLQSGVPSRF
SGSGSGTDFTLTISSLQPEDFATYYCQQGARWPQTFGQGTKVEIKRHH
HHHH

9) dsbA signal sequence-wildtype Fn3 (RGD to RGA) sequence-GS linker-anti-RSA-His tag (pQE-80L)

(SEQ ID NO: 92)
MKKIWLALAGLVLAFSASAGSVSDVPRDLEVVAATPTSLLISWDAPAV
TVRYYRITYGETGGNSPVQEFTVPGSKSTATISGLKPGVDYTITVYAV
TGRGASPASSKPISINYRTGGGGSGGGGSGGGGSDIQMTQSPSSLSAS
VGDRVTITCRASQSISSYLNWYQQKPGKAPKLLIYRNSPLQSGVPSRF
SGSGSGTDFTLTISSLQPEDFATYYCQQTYRVPPTFGQGTKVEIKRHH
HHHH

The ligation mix was used to transform XL1-Blue or DH5alpha competent cells. Positive clones were verified by DNA sequencing. Constructs were expressed in several E. coli strains including KS474 and TG1 (−). After induction and expression, cell pellets were frozen at −20° C. and then resuspended in lysis buffer (20 mM NaH₂PO₄, 10 mM Imidazol, 500 mM NaCl, 1 tablet Complete without EDTA per 50 ml buffer (Roche), 2 mM MgCl₂, 10 U/ml Benzonase (Merck) [pH7.4]. Cells were sonicated on ice and centrifuged. Supernatant was filtered and loaded onto a Ni-NTA column. The column was washed with Wash buffer (as for lysis buffer but with 20 mM Imidazol) and then eluted with Elution Buffer (as for lysis buffer but up to 500 mM Imidazol). Samples were analysed on Bis-Tris Gels (Invitrogen), then concentrated in Amicon Ultra-15 tubes, loaded onto a Superdex prep grade column (Amersham) and eluted with 10 mM Tris buffer or PBS. 100K Amicon centrifugal filters were used for endotoxin removal. Samples were analysed again on Bis-Tris gels and by LC-MS. Binding to corresponding antigen was verified by ELISA. The half-life of these constructs was determined in vivo. 10 mg/kg of each compound was administered intravenously into Lewis rats (n=3), samples were taken at pre-dose, 1 2, 4, 8, 24, 48, 96, 192 and 384 hrs. Biacore analysis was performed using a CM5 chip with standard amine coupling. Flow cell 1 was blank (surface activation with EDC/NHS and subsequent deactivation with Ethanolamine) for reference subtraction. Flow cell 2 was coated with HSA (Fluka) for PK read-out. Flow cells 3 and 4 were coated with compounds that were administered to the animals (surface saturation) for immunogenicity read-out. Rat serum samples were diluted 1:8 with HBS-EP and NBSreducer (Biacore; final conc. 1 mg/ml). A standard curve was prepared for compound quantification, a 1:2 dilution series from 20 mg/l down to 0.078 mg/l of the corresponding compound that was administered to the animals was prepared in rat serum (GeneTex). The rat serum was diluted 1:8 with HBS-EP and 1 mg/ml NSBreducer. The standard curve data were fitted using XLfit 4.2 and used to calculate the compound concentrations in the serum samples (PK). The compound half-life was calculated using the WinNonlin software. PK data were fitted using a non-compartmental model. The results of the study are described below.

(ii) Serum Albumin Fusion Molecules

The DNA sequences corresponding to the CD33 SS-TNF-binding Fn3 sequence (SEQ ID NO: 6), CD33 SS-TNF-binding Fn3 (R18L & I56T) (SEQ ID NO: 7), CD33 SS-wildtype Fn3 sequence (SEQ ID NO: 8) and CD33 SS-wildtype Fn3 (RGD to RGA) (SEQ ID NO: 9) were optimised for expression in mammalian cells and prepared at Geneart AG, Germany. The resulting DNA fragments were ligated into pRS5a using BlpI/XbaI (appropriate flanking DNA sequences such as Kozak were added to vector). HSA was amplified by PCR using primers 1 (SEQ ID NO: 16) and 2 (SEQ ID NO: 17) (primer 2 encodes a His tag) and inserted into pRS5a (CD33-TNF-binding Fn3 sequences (SEQ ID NO: 6 and SEQ ID NO: 7) or CD33-wildtype Fn3 sequences (SEQ ID NO: 8 and SEQ ID NO: 9) using RsrII/XbaI. RSA was amplified by PCR from vector IRBPp993CO328D (RZPD) using primers 26 (SEQ ID NO: 86) and 27 (SEQ ID NO: 87), and then cloned into pRS5a-CD33 signal sequence-wild type Fn3 (RGD to RGA)-HSA-His (SEQ ID NO: 99) via RsrII/XbaI. I431V was integrated by site directed mutagenesis using primers 28 (SEQ ID NO: 88) and 29 (SEQ ID NO: 89), L262V was integrated by site-directed mutagenesis using primers 30 (SEQ ID NO: 90) and 31 (SEQ ID NO: 91). The DNA sequence for the VEGFR-binding Fn3 (SEQ ID NO: 77) was optimized for expression in mammalian cells and prepared at Geneart AG, Germany. The DNA was digested with RsRII/CelII and cloned into the corresponding sites of pRS5a-CD33 signal sequence-wildtype Fn3 (RGD to RGA)-HSA-His (SEQ ID NO: 99. RSA was isolated from vector pRS5a-CD33 signal sequence-wildtype Fn3 (RGD to RGA)-RSA-His (SEQ ID NO: 100) and cloned into pRS5a-CD33 signal sequence-VEGFR binding Fn3-HSA-His (SEQ ID NO: 101) via RsrII/XbaI.

### Formats:

1) CD33 signal sequence-TNF-binding Fn3 sequence-HSA-His tag (pRS5a)

(SEQ ID NO: 96)
MPLLLLLPLLWAGALAVSDVPRDLEVVAATPTSRLISWNRSGLQSRYY
RITYGETGGNSPVQEFTVPPWASIATISGLKPGVDYTITVYAVTDKSD
TYKYDDPISINYRTDAHKSEVAHRFKDLGEENFKALVLIAFAQYLQQC
PFEDHVKLVNEVTEFAKTCVADESAENCDKSLHTLFGDKLCTVATLRE
TYGEMADCCAKQEPERNECFLQHKDDNPNLPRLVRPEVDVMCTAFHDN
EETFLKKYLYEIARRHPYFYAPELLFFAKRYKAAFTECCQAADKAACL
LPKLDELRDEGKASSAKQRLKCASLQKFGERAFKAWAVARLSQRFPKA
EFAEVSKLVTDLTKVHTECCHGDLLECADDRADLAKYICENQDSISSK
LKECCEKPLLEKSHCIAEVENDEMPADLPSLAADFVESKDVCKNYAEA
KDVFLGMFLYEYARRHPDYSVVLLLRLAKTYETTLEKCCAAADPHECY
AKVFDEFKPLVEEPQNLIKQNCELFEQLGEYKFQNALLVRYTKKVPQV
STPTLVEVSRNLGKVGSKCCKHPEAKRMPCAEDYLSVVLNQLCVLHEK
TPVSDRVTKCCTESLVNRRPCFSALEVDETYVPKEFNAETFTFHADIC
TLSEKERQIKKQTALVELVKHKPKATKEQLKAVMDDFAAFVEKCCKAD
DKETCFAEEGKKLVAASQAALGLHHHHHH

2) CD33 signal sequence-TNF-binding Fn3 (R18L & I56T) sequence-HSA-His tag (pRS5a)

(SEQ ID NO: 97)
MPLLLLLPLLWAGALAVSDVPRDLEVVAATPTSLLISWNRSGLQSRYY
RITYGETGGNSPVQEFTVPPWASTATISGLKPGVDYTITVYAVTDKSD
TYKYDDPISINYRTDAHKSEVAHRFKDLGEENFKALVLIAFAQYLQQC
PFEDHVKLVNEVTEFAKTCVADESAENCDKSLHTLFGDKLCTVATLRE
TYGEMADCCAKQEPERNECFLQHKDDNPNLPRLVRPEVDVMCTAFHDN
EETFLKKYLYEIARRHPYFYAPELLFFAKRYKAAFTECCQAADKAACL
LPKLDELRDEGKASSAKQRLKCASLQKFGERAFKAWAVARLSQRFPKA
EFAEVSKLVTDLTKVHTECCHGDLLECADDRADLAKYICENQDSISSK
LKECCEKPLLEKSHCIAEVENDEMPADLPSLAADFVESKDVCKNYAEA
KDVFLGMFLYEYARRHPDYSVVLLLRLAKTYETTLEKCCAAADPHECY
AKVFDEFKPLVEEPQNLIKQNCELFEQLGEYKFQNALLVRYTKKVPQV
STPTLVEVSRNLGKVGSKCCKHPEAKRMPCAEDYLSVVLNQLCVLHEK
TPVSDRVTKCCTESLVNRRPCFSALEVDETYVPKEFNAETFTFHADIC
TLSEKERQIKKQTALVELVKHKPKATKEQLKAVMDDFAAFVEKCCKAD
DKETCFAEEGKKLVAASQAALGLHHHHHH

3) CD33 signal sequence-wildtype Fn3 sequence-HSA-His tag (pRS5a)

(SEQ ID NO: 98)
MPLLLLLPLLWAGALAVSDVPRDLEVVAATPTSRLISWDAPAVTVRYY
RITYGETGGNSPVQEFTVPGSKSIATISGLKPGVDYTITVYAVTGRGD
SPASSKPISINYRTDAHKSEVAHRFKDLGEENFKALVLIAFAQYLQQC
PFEDHVKLVNEVTEFAKTCVADESAENCDKSLHTLFGDKLCTVATLRE
TYGEMADCCAKQEPERNECFLQHKDDNPNLPRLVRPEVDVMCTAFHDN
EETFLKKYLYEIARRHPYFYAPELLFFAKRYKAAFTECCQAADKAACL
LPKLDELRDEGKASSAKQRLKCASLQKFGERAFKAWAVARLSQRFPKA
EFAEVSKLVTDLTKVHTECCHGDLLECADDRADLAKYICENQDSISSK
LKECCEKPLLEKSHCIAEVENDEMPADLPSLAADFVESKDVCKNYAEA
KDVFLGMFLYEYARRHPDYSVVLLLRLAKTYETTLEKCCAAADPHECY
AKVFDEFKPLVEEPQNLIKQNCELFEQLGEYKFQNALLVRYTKKVPQV
STPTLVEVSRNLGKVGSKCCKHPEAKRMPCAEDYLSVVLNQLCVLHEK
TPVSDRVTKCCTESLVNRRPCFSALEVDETYVPKEFNAETFTFHADIC
TLSEKERQIKKQTALVELVKHKPKATKEQLKAVMDDFAAFVEKCCKAD
DKETCFAEEGKKLVAASQAALGLHHHHHH

4) CD33 signal sequence-wildtype Fn3 (RGD to RGA) sequence-HSA-His tag (pRS5a)

(SEQ ID NO: 99)
MPLLLLLPLLWAGALAVSDVPRDLEVVAATPTSRLISWDAPAVTVRYY
RITYGETGGNSPVQEFTVPGSKSIATISGLKPGVDYTITVYAVTGRGA
SPASSKPISINYRTDAHKSEVAHRFKDLGEENFKALVLIAFAQYLQQC
PFEDHVKLVNEVTEFAKTCVADESAENCDKSLHTLFGDKLCTVATLRE
TYGEMADCCAKQEPERNECFLQHKDDNPNLPRLVRPEVDVMCTAFHDN
EETFLKKYLYEIARRHPYFYAPELLFFAKRYKAAFTECCQAADKAACL
LPKLDELRDEGKASSAKQRLKCASLQKFGERAFKAWAVARLSQRFPKA
EFAEVSKLVTDLTKVHTECCHGDLLECADDRADLAKYICENQDSISSK
LKECCEKPLLEKSHCIAEVENDEMPADLPSLAADFVESKDVCKNYAEA
KDVFLGMFLYEYARRHPDYSVVLLLRLAKTYETTLEKCCAAADPHECY
AKVFDEFKPLVEEPQNLIKQNCELFEQLGEYKFQNALLVRYTKKVPQV
STPTLVEVSRNLGKVGSKCCKHPEAKRMPCAEDYLSVVLNQLCVLHEK
TPVSDRVTKCCTESLVNRRPCFSALEVDETYVPKEFNAETFTFHADIC
TLSEKERQIKKQTALVELVKHKPKATKEQLKAVMDDFAAFVEKCCKAD
DKETCFAEEGKKLVAASQAALGLHHHHHH

5) CD33 signal sequence-wildtype Fn3 (RGD to RGA) sequence-RSA-His tag (pRS5a)

(SEQ ID NO: 100)
MPLLLLLPLLWAGALAVSDVPRDLEVVAATPTSLLISWDAPAVTVRYY
RITYGETGGNSPVQEFTVPGSKSTATISGLKPGVDYTITVYAVTGRGA
SPASSKPISINYRTEAHKSEIAHRFKDLGEQHFKGLVLIAFSQYLQKC
PYEEHIKLVQEVTDFAKTCVADENAENCDKSIHTLFGDKLCAIPKLRD
NYGELADCCAKQEPERNECFLQHKDDNPNLPPFQRPEAEAMCTSFQEN
PTSFLGHYLHEVARRHPYFYAPELLYYAEKYNEVLTQCCTESDKAACL
TPKLDAVKEKALVAAVRQRMKCSSMQRFGERAFKAWAVARMSQRFPNA
EFAEITKLATDVTKINKECCHGDLLECADDRAELAKYMCENQATISSK
LQACCDKPVLQKSQCLAEIEHDNIPADLPSIAADFVEDKEVCKNYAEA
KDVFLGTFLYEYSRRHPDYSVSLLLRLAKKYEATLEKCCAEGDPPACY
GTVLAEFQPLVEEPKNLVKTNCELYEKLGEYGFQNAVLVRYTQKAPQV
STPTLVEAARNLGRVGTKCCTLPEAQRLPCVEDYLSAILNRLCVLHEK
TPVSEKVTKCCSGSLVERRPCFSALTVDETYVPKEFKAETFTFHSDIC
TLPDKEKQIKKQTALAELVKHKPKATEDQLKTVMGDFAQFVDKCCKAA
DKDNCFATEGPNLVARSKEALAHHHHHH

6) CD33 signal sequence-VEGFR-binding Fn3-HSA-His tag (pRS5a)

(SEQ ID NO: 101)
MPLLLLLPLLWAGALAGEVVAATPTSLLISWRHPHFPTRYYRITYGET
GGNSPVQEFTVPLQPPTATISGLKPGVDYTITVYAVTDGRNGRLLSIP
ISINYRTDAHKSEVAHRFKDLGEENFKALVLIAFAQYLQQCPFEDHVK
LVNEVTEFAKTCVADESAENCDKSLHTLFGDKLCTVATLRETYGEMAD
CCAKQEPERNECFLQHKDDNPNLPRLVRPEVDVMCTAFHDNEETFLKK
YLYEIARRHPYFYAPELLFFAKRYKAAFTECCQAADKAACLLPKLDEL
RDEGKASSAKQRLKCASLQKFGERAFKAWAVARLSQRFPKAEFAEVSK
LVTDLTKVHTECCHGDLLECADDRADLAKYICENQDSISSKLKECCEK
PLLEKSHCIAEVENDEMPADLPSLAADFVESKDVCKNYAEAKDVFLGM
FLYEYARRHPDYSVVLLLRLAKTYETTLEKCCAAADPHECYAKVFDEF
KPLVEEPQNLIKQNCELFEQLGEYKFQNALLVRYTKKVPQVSTPTLVE
VSRNLGKVGSKCCKHPEAKRMPCAEDYLSVVLNQLCVLHEKTPVSDRV
TKCCTESLVNRRPCFSALEVDETYVPKEFNAETFTFHADICTLSEKER
QIKKQTALVELVKHKPKATKEQLKAVMDDFAAFVEKCCKADDKETCFA
EEGKKLVAASQAALGLHHHHHH

7) CD33 signal sequence-VEGFR-binding Fn3-RSA-His tag (pRS5a)

(SEQ ID NO: 102)
MPLLLLLPLLWAGALAGEVVAATPTSLLISWRHPHFPTRYYRITYGET
GGNSPVQEFTVPLQPPTATISGLKPGVDYTITVYAVTDGRNGRLLSIP
ISINYRTEAHKSEIAHRFKDLGEQHFKGLVLIAFSQYLQKCPYEEHIK
LVQEVTDFAKTCVADENAENCDKSIHTLFGDKLCAIPKLRDNYGELAD
CCAKQEPERNECFLQHKDDNPNLPPFQRPEAEAMCTSFQENPTSFLGH
YLHEVARRHPYFYAPELLYYAEKYNEVLTQCCTESDKAACLTPKLDAV
KEKALVAAVRQRMKCSSMQRFGERAFKAWAVARMSQRFPNAEFAEITK
LATDVTKINKECCHGDLLECADDRAELAKYMCENQATISSKLQACCDK
PVLQKSQCLAEIEHDNIPADLPSIAADFVEDKEVCKNYAEAKDVFLGT
FLYEYSRRHPDYSVSLLLRLAKKYEATLEKCCAEGDPPACYGTVLAEF
QPLVEEPKNLVKTNCELYEKLGEYGFQNAVLVRYTQKAPQVSTPTLVE
AARNLGRVGTKCCTLPEAQRLPCVEDYLSAILNRLCVLHEKTPVSEKV
TKCCSGSLVERRPCFSALTVDETYVPKEFKAETFTFHSDICTLPDKEK
QIKKQTALAELVKHKPKATEDQLKTVMGDFAQFVDKCCKAADKDNCFA
TEGPNLVARSKEALAHHHHHH

The ligation mix was used to transform XL1-Blue or DH5alpha competent cells. Positive clones were verified by DNA sequencing. Constructs were expressed in several cell-lines including HEK293T, FreeStyle™ 293-F, HKB11 and HEKEBNA. Endotoxin ‘free’ buffers were used for all steps. Culture supernatants were filtered and loaded onto a Ni-NTA column. Column was washed with Wash buffer (20 mM NaH₂PO₄, 20 mM Imidazol, 500 mM NaCl, 1 tablet Complete without EDTA per 50 ml buffer (Roche), 2 mM MgCl₂, 10 U/ml Benzonase (Merck) [pH7.4]) and then eluted with Elution Buffer (as for Wash buffer but up to 500 mM Imidazol). Samples were analysed on Bis-Tris Gels (Invitrogen), then concentrated in Amicon Ultra-15 tubes, loaded onto a Superdex prep grade column (Amersham) and eluted with 10 mM Tris buffer or PBS. Samples were analysed again on Bis-Tris gels and by LC-MS. Binding to corresponding antigen was verified by ELISA. The half-life of these constructs was determined in vivo. 10 mg/kg of each compound was administered intravenously into Lewis rats (n=3), samples were taken at pre-dose, 1 2, 4, 8, 24, 48, 96, 192 and 384 hrs. Biacore analysis was performed using a CM5 chip with standard amine coupling. Flow cell 1 was blank (surface activation with EDC/NHS and subsequent deactivation with Ethanolamine) for reference subtraction. Flow cell 2 was coated with THE anti-HIS mAb (GenScript Corp) for PK read-out. Flow cells 3 and 4 were coated with compounds that were administered to the animals (surface saturation) for immunogenicity read-out. Rat serum samples were diluted 1:8 with HBS-EP and NBSreducer (Biacore; final conc. 1 mg/ml). A standard curve was prepared for compound quantification, a 1:2 dilution series from 20 mg/l down to 0.078 mg/l of the corresponding compound that was administered to the animals was prepared in rat serum (GeneTex). The rat serum was diluted 1:8 with HBS-EP and 1 mg/ml NSBreducer. The standard curve data were fitted using XLfit 4.2 and used to calculate the compound concentrations in the serum samples (PK). The compound half-life was calculated using the WinNonlin software. PK data were fitted using a non-compartmental model.

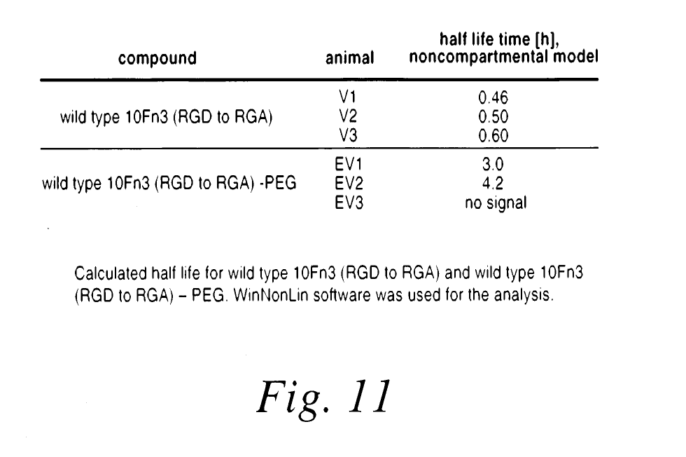

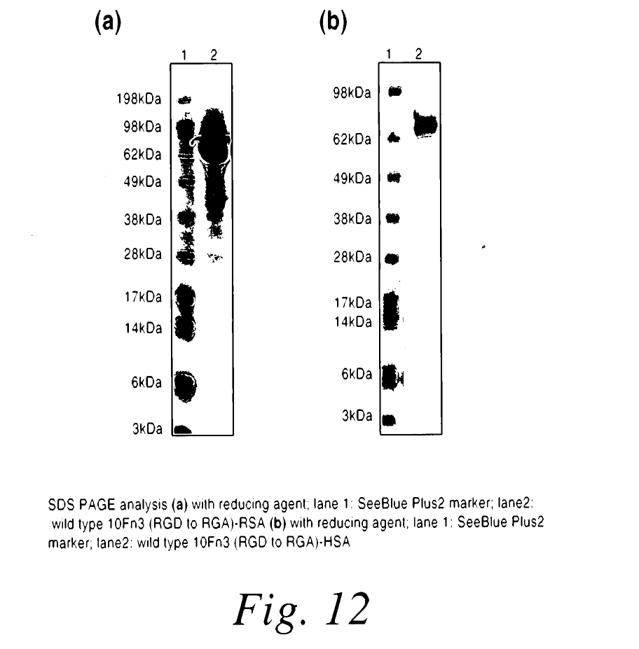

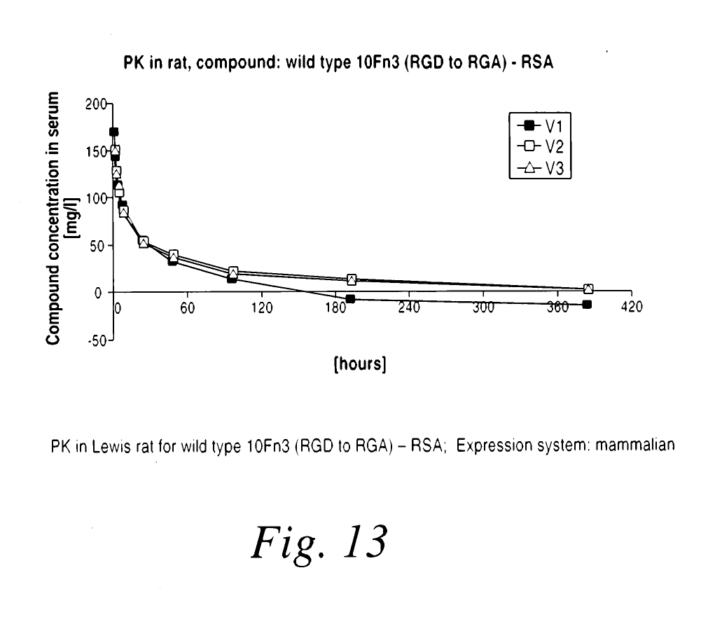

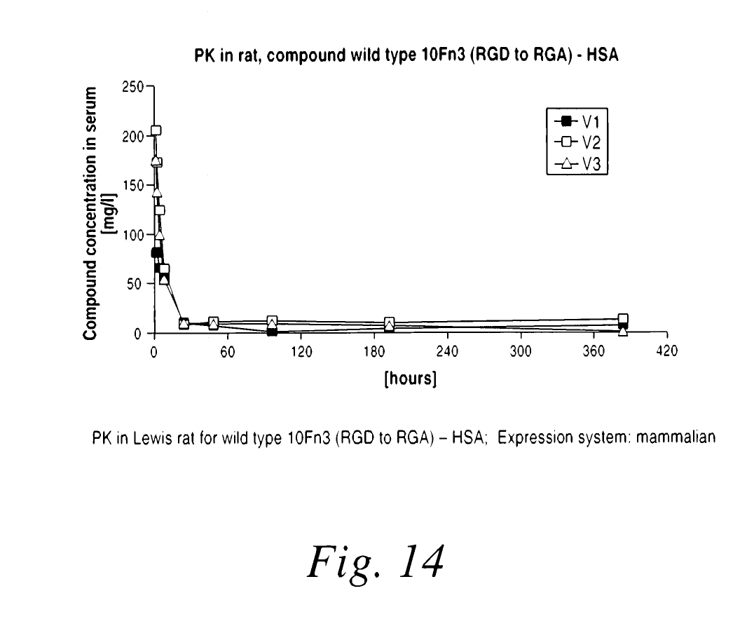

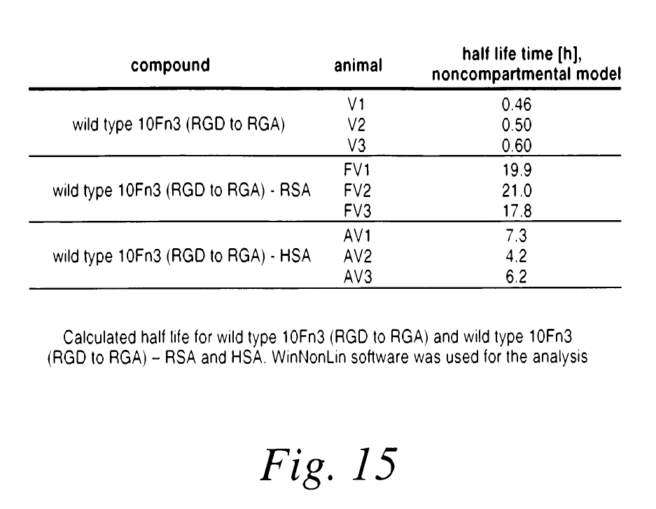

Wild type 10Fn3 (RGD to RGA)-RSA and HSA fusions were expressed in mammalian cells, purified and analysed by SDS-PAGE (FIG. 12). LC-MS showed a mass of 76.62 kDa and 77.17 kDa for wild type 10Fn3 (RGD to RGA)-RSA and wild type 10Fn3 (RGD to RGA)-HSA respectively after reduction corresponding to the correct proteins (data not shown). N-terminal analysis also showed a sequence corresponding to the expected protein. In vivo data showed a significant half-life improvement for both wild type 10Fn3 (RGD to RGA) RSA and HSA fusions (FIGS. 13 and 14) when compared with unmodified 10Fn3 (FIG. 9). The average half-life for unmodified 10Fn3 was 0.52 h, this increased to 19.6 h for 10Fn3-RSA and to 5.9 h for 10Fn3-HSA (FIG. 15). The half-life for 10Fn3-HSA was lower when compared with 10Fn3-RSA in rat. This could be due to the possibility that HSA does not efficiently bind to Lewis rat FcRn.

Using Formula 1, the extrapolated average half-life in man is expected to be about 80.9 hours.

The average fold increase of half life with the RSA conjugated Fn3 molecule is the average Fn3-RSA conjugate (19.6) divided by average unconjugated Fn3 (0.52), resulting in approximately 38 fold increase in half-life of the Fn3-RSA conjugate in vivo. This is expected to extrapolate in man using HSA.

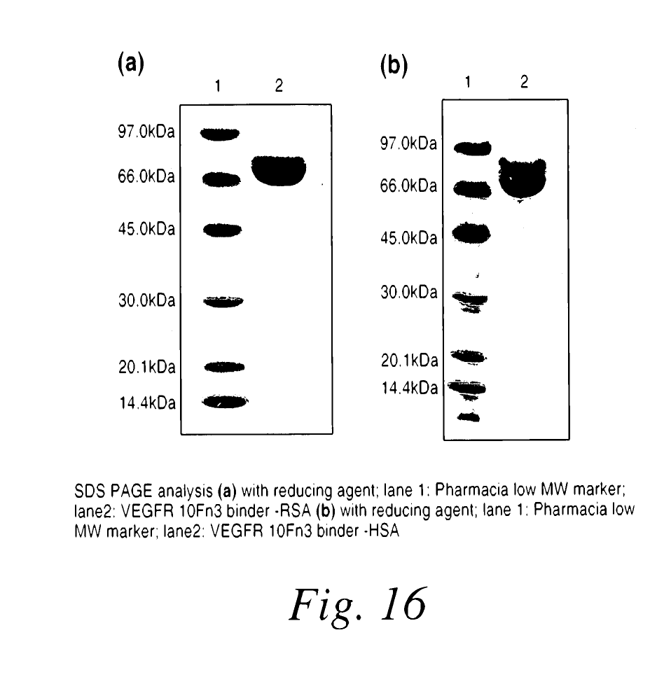

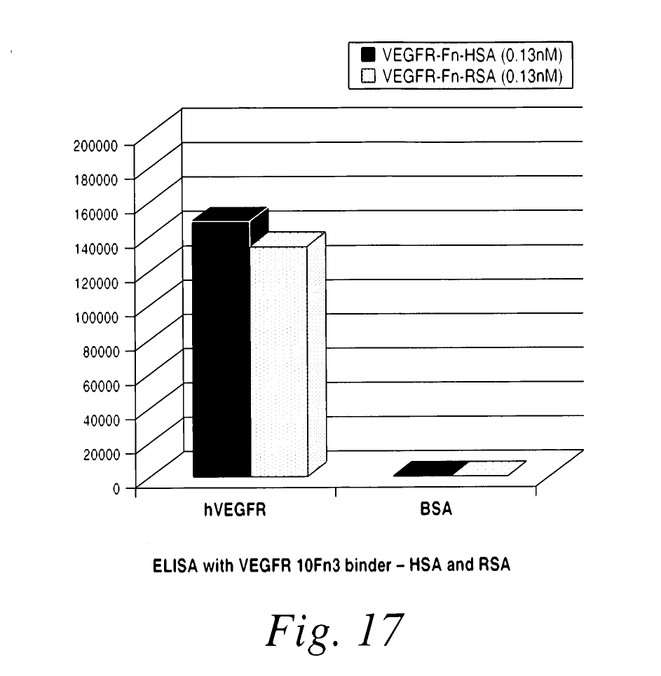

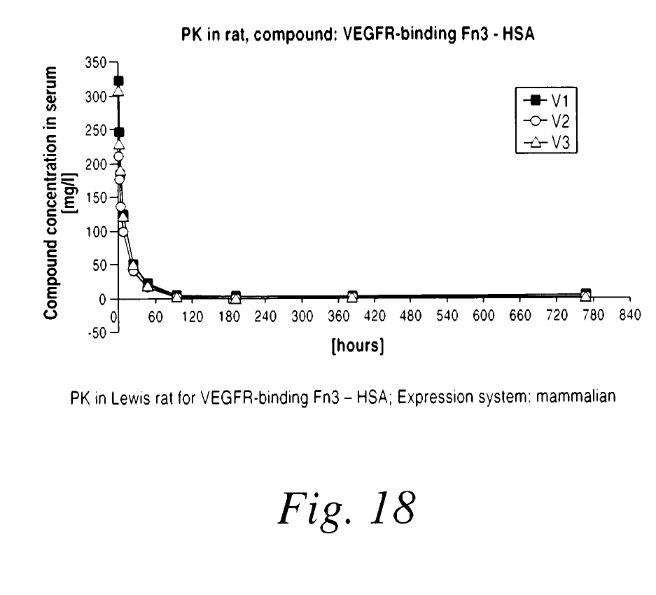

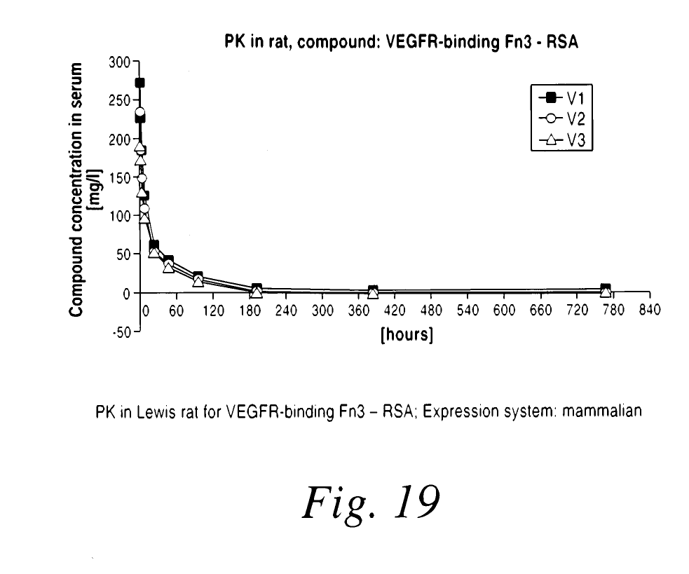

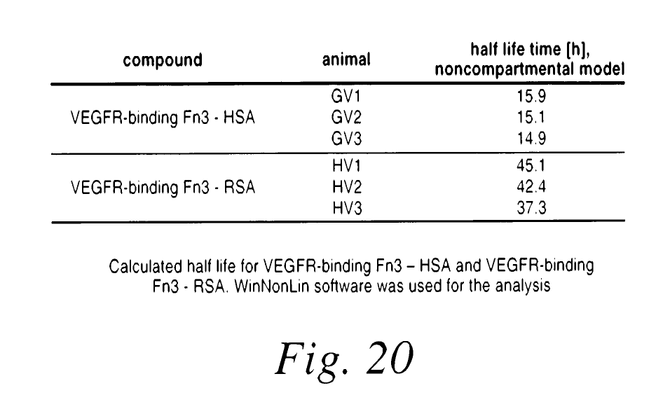

VEGFR-binding Fn3-RSA and HSA fusions were also expressed in mammalian cells, purified and analysed by SDS-PAGE (FIG. 16). LC-MS showed a mass of 76.27 kDa and 76.82 kDa for VEGFR-binding Fn3-RSA and VEGFR-binding-HSA respectively, these molecular weights corresponded to the expected proteins (data not shown). Specific binding to hVEGFR was confirmed by ELISA for both HSA and RSA fusions (FIG. 17). The average half-lives for the RSA) (FIG. 18) and HSA (FIG. 19) fusions were 41.6 h and 15.3 h respectively (FIG. 20).

With a therapeutic Fn3, e.g., VEGFR-binding Fn3-RSA, the extrapolated average half-life in man is expected to be about 172 hours.

The average fold increase of half life of this conjugated Fn3 molecule is the average VEGFR-binding Fn3-RSA conjugate (41.6) divided by average unconjugated Fn3 (0.52), resulting in approximately 80 fold increase in half-life of the Fn3-RSA conjugate in vivo. This is expected to extrapolate in man using HSA (data not shown).

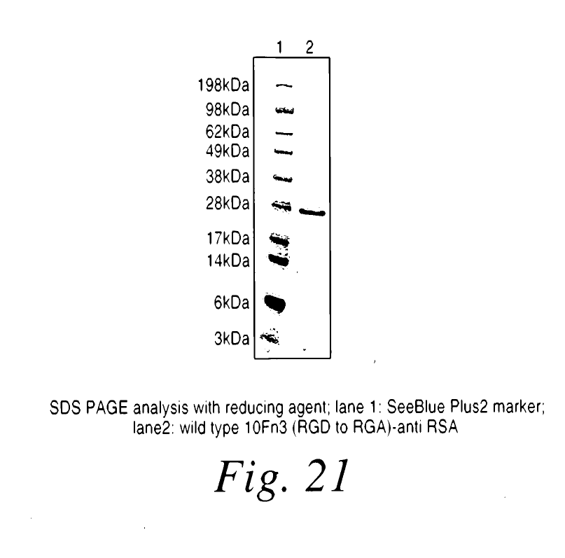

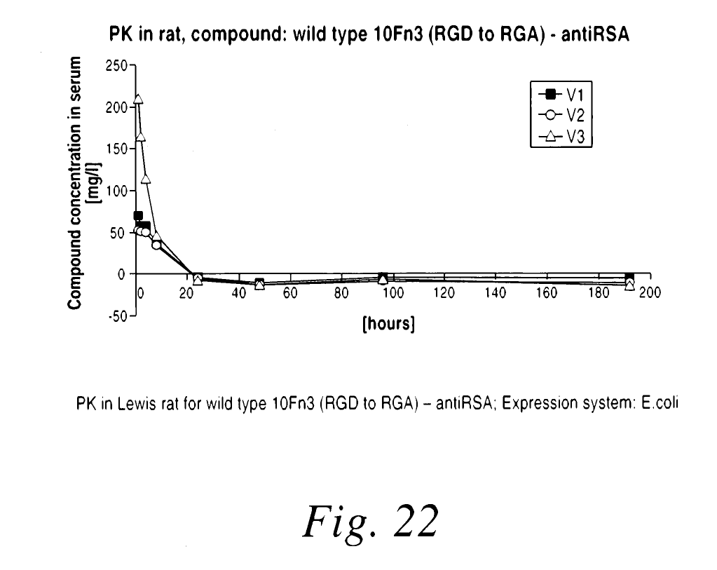

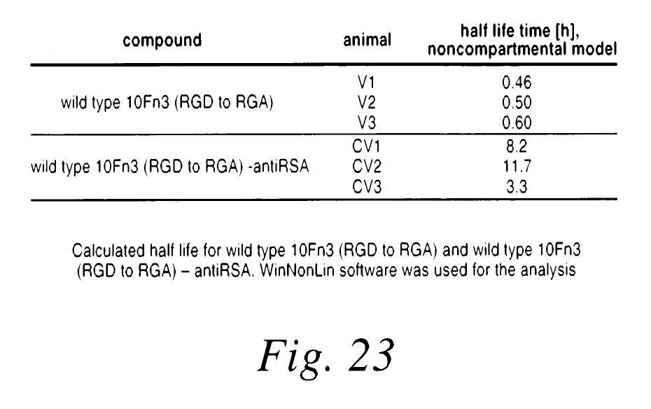

Wild type 10Fn3 (RGD to RGA) anti-RSA was expressed in E. coli, purified and analysed by SDS-PAGE (FIG. 21). LC-MS showed a mass of 23.68 kDa corresponding to the correct protein (data not shown). In vivo data showed a significant half-life improvement for the anti-RSA fusion (FIG. 22) when compared with unmodified 10Fn3 (FIG. 9). The average half-life for unmodified 10Fn3 was 0.52 h, this increased to 7.7 h for 10Fn3-antiRSA (FIG. 23).

The results of this rat study demonstrate that the in vivo serum half-life of 10Fn3 can be significantly extended when prepared as a fusion to serum albumin or to a serum albumin binder.

Using Formula 1, the extrapolated average half-life in man is expected to be about 31.8 hours.

The average fold increase of half life with the anti-HSA conjugated Fn3 molecule is the average Fn3-anti-HSA conjugate (7.7) divided by average unconjugated Fn3 (0.52), resulting in approximately 15 fold increase in half-life of the Fn3-anti-HSA conjugate in vivo.

### Example 5

### Fc—Fibronectin Fusions

The DNA sequences corresponding to the CD33 SS-TNF-binding Fn3 sequence (SEQ ID NO:6), CD33 SS-TNF-binding Fn3 (R18L and I56T) (SEQ ID NO:7), CD33 SS-wildtype Fn3 sequence (SEQ ID NO:8) and CD33 SS-wildtype Fn3 (RGD to RGA) (SEQ ID NO:9) were optimised for expression in mammalian cells and prepared at Geneart AG, Germany. The resulting DNA fragments were ligated into pRS5a using BlpI/XbaI (appropriate flanking DNA sequences such as Kozak were added to vector). hIgG1 Fc was amplified by PCR using primers 18 (SEQ ID NO: 33) and 19 (SEQ ID NO: 34) (primer 19 encodes a His tag) and inserted into pRS5a (CD33-TNF-binding Fn3 sequences (SEQ ID NO: 6 and SEQ ID NO: 7) or CD33-wildtype Fn3 sequences (SEQ ID NO: 8 and SEQ ID NO: 9) using RsrII/XbaI.

### Formats:

1) CD33 signal sequence-TNF-binding Fn3 sequence-Fc-His tag (pRS5a)

(SEQ ID NO:72)
MPLLLLLPLLWAGALAVSDVPRDLEVVAATPTSRLISWNRSGLQSRYY
RITYGETGGNSPVQEFTVPPWASIATISGLKPGVDYTITVYAVTDKSD
TYKYDDPISINYRTGKSCDKTHTCPPCPAPELLGGPSVFLFPPKPKDT
LMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREP
QVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLS
LSPGKHHHHHH

2) CD33 signal sequence-TNF-binding Fn3 (R18L and I56T) sequence-Fc-His tag (pRS5a)

(SEQ ID NO: 73)
MPLLLLLPLLWAGALAVSDVPRDLEVVAATPTSLLISWNRSGLQSRYY
RITYGETGGNSPVQEFTVPPWASTATISGLKPGVDYTITVYAVTDKSD
TYKYDDPISINYRTGKSCDKTHTCPPCPAPELLGGPSVFLFPPKPKDT
LMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREP
QVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLS
LSPGKHHHHHH

3) CD33 signal sequence-wildtype Fn3 sequence-Fc-His tag (pRS5a)

(SEQ ID NO: 74)
MPLLLLLPLLWAGALAVSDVPRDLEVVAATPTSLLISWDAPAVTVRYY
RITYGETGGNSPVQEFTVPGSKSTATISGLKPGVDYTITVYAVTGRGD
SPASSKPISINYRTGKSCDKTHTCPPCPAPELLGGPSVFLFPPKPKDT
LMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREP
QVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLS
LSPGKHHHHHH

4) CD33 signal sequence-wildtype Fn3 (RGD to RGA) sequence-Fc-His tag (pRS5a)

(SEQ ID NO: 75)
MPLLLLLPLLWAGALAVSDVPRDLEVVAATPTSLLISWDAPAVTVRYY
RITYGETGGNSPVQEFTVPGSKSTATISGLKPGVDYTITVYAVTGRGA
SPASSKPISINYRTGKSCDKTHTCPPCPAPELLGGPSVFLFPPKPKDT
LMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREP
QVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLS
LSPGKHHHHHH

The ligation mix was used to transform XL1-Blue or DH5alpha competent cells. Positive clones were verified by DNA sequencing. Constructs were expressed in several cell-lines including HEK293T, FreeStyle™ 293-F, HKB11 and HEKEBNA. Endotoxin ‘free’ buffers were used for all steps. Culture supernatants were filtered and loaded onto a Protein A Sepharose column. Column was washed with PBS and then eluted with 50 mM citrate, pH2.7, 140 mM NaCl. Samples were neutralised and analysed on Bis-Tris Gels (Invitrogen), then concentrated in Amicon Ultra-15 tubes, loaded onto a Superdex prep grade column (Amersham) and eluted with 10 mM Tris buffer or PBS. Samples were analysed again on Bis-Tris gels and by LC-MS. For reduction and N-deglycosylation, samples (34 μg) were incubated in a final volume of 50 μl with 0.8M urea, 0.04M NH₄CO₃ and 0.01M DTT for 30 mins at 50° C. 1× reaction buffer G7 and 1 μg of PNGaseF were then added and incubated for 1 h at 37° C. In addition to Protein A purification, Ni-NTA purification was also conducted as described in previous examples. Binding to corresponding antigen was verified by ELISA.

The half-life of these constructs was determined in vivo. 10 mg/kg of each compound was administered intravenously into Lewis rats (n=3), samples were taken at pre-dose, 1 2, 4, 8, 24, 48, 96, 192 and 384 hrs. Biacore analysis was performed using a CM5 chip with standard amine coupling. Flow cell 1 was blank (surface activation with EDC/NHS and subsequent deactivation with Ethanolamine) for reference subtraction. Flow cell 2 was coated with THE anti-HIS mAb (GenScript Corp) for PK read-out. Flow cells 3 and 4 were coated with compounds that were administered to the animals (surface saturation) for immunogenicity read-out. Rat serum samples were diluted 1:8 with HBS-EP and NBSreducer (Biacore; final conc. 1 mg/ml). A standard curve was prepared for compound quantification, a 1:2 dilution series from 20 mg/l down to 0.078 mg/l of the corresponding compound that was administered to the animals was prepared in rat serum (GeneTex). The rat serum was diluted 1:8 with HBS-EP and 1 mg/ml NSBreducer. The standard curve data were fitted using XLfit 4.2 and used to calculate the compound concentrations in the serum samples (PK). The compound half-life was calculated using the WinNonlin software. PK data were fitted using a non-compartmental model.

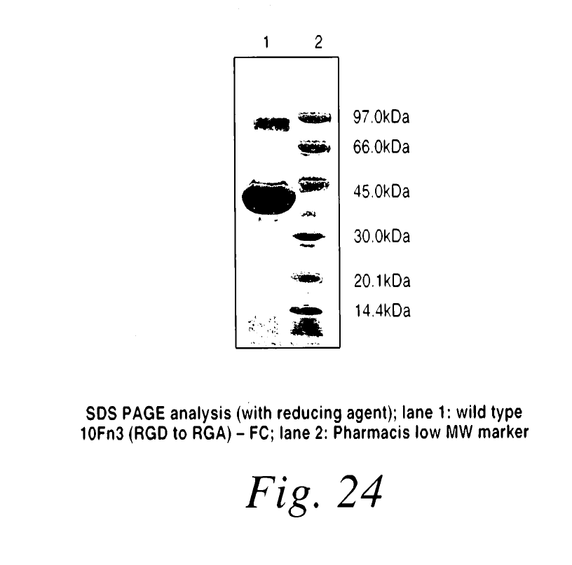

Wild type 10Fn3 (RGD to RGA)-Fc was expressed in mammalian cells, purified and analysed by SDS-PAGE (FIG. 24). LC-MS showed different forms for native wild type 10Fn3 (RGD to RGA)-Fc, the 76.12 kDa mass corresponded to a dimer, the 76.28 kDa and 76.44 kDa forms corresponded to dimer plus hexose. After reduction and N-deglycosylation, a mass of 36.63 kDa was obtained which corresponded to the expected monomeric protein (data not shown). The MW of the protein increased after deglycosylation due to the mass difference from modification of Asn to Asp during N-deglycosylation. N-terminal analysis also showed a sequence corresponding to the expected protein.

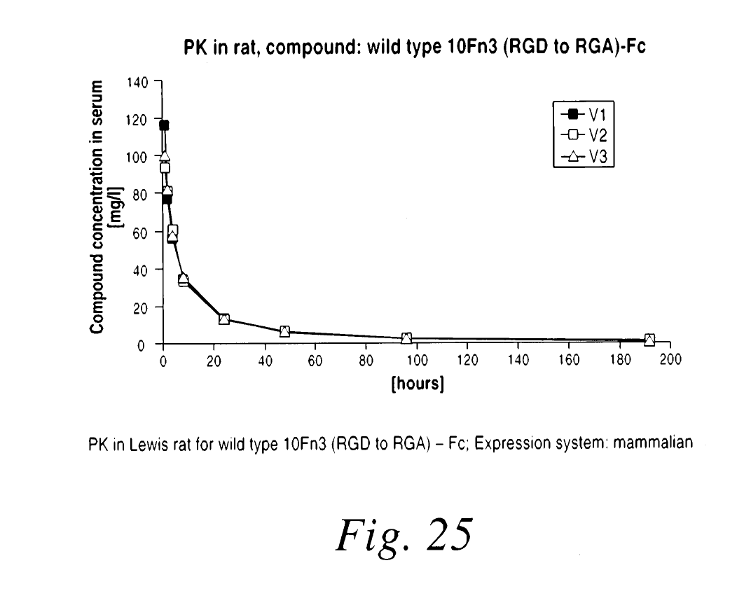

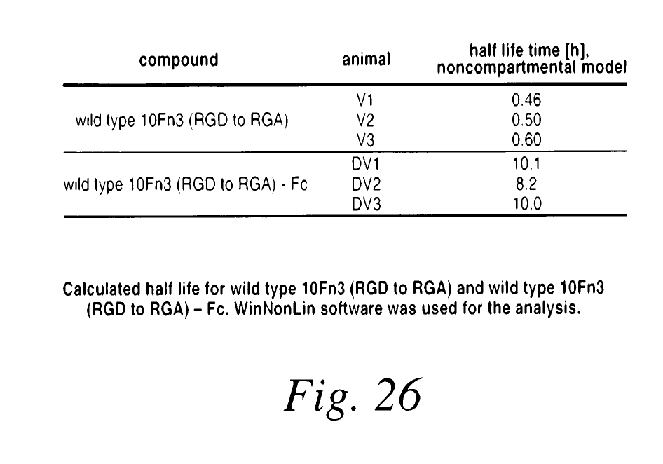

In vivo data showed a significant half-life improvement for wild type 10Fn3 (RGD to RGA)-Fc (FIG. 25) when compared with unmodified 10Fn3 (FIG. 9). The average half-life for unmodified 10Fn3 was 0.52 h, this increased to 9.4 h for 10Fn3-Fc (FIG. 26).

The results of this rat study demonstrate that the in vivo serum half-life of 10Fn3 can be significantly extended when prepared as a fusion to hIgG1 Fc.

Using Formula 1, the extrapolated average half-life in man is expected to be about 38.8 hours.

The average fold increase of half life with Fc fused to Fn3 molecule is the average Fn3-Fc fusion (9.4) divided by average unconjugated Fn3 (0.52), resulting in approximately 18 fold increase in half-life of the Fn3-Fc fusion in vivo.

Collectively, the results in Examples 3-5 show that the Fn3 molecule can be modified to increase its half-life of the molecule by a number of methods, e.g., HSA, Fc fusion. All the modified Fn3 molecules demonstrated a marked increase in half-life, Furthermore, these examples demonstrate for the first time that Fn3 and modified forms of Fn3 can be successfully expressed in vivo in mammalian cells and have a significant in vivo effect on clearance.

### Example 6

### Chimeric Fibronectin Molecules

Using the type III module of fibronectin and the sequence analysis of the beta-strands described in U.S. Pat. No. 6,673,901 B2, methods for swapping fibronectin strands to produce chimeric Fn3 molecules are described here.

First, the beta strands of domains 7, 8, 9, and 10 were identified. Residues which are involved in the hydrophobic core interactions were then identified. Similarities according to the following criteria was then determined:

(a) similarity among the strands;
(b) similarity among only the positions defined as involved in hydrophobic core interactions; and
(c) similarity among the positions which are not involved in hydrophobic interactions but solvent exposed.

With reference to the table below, the % identity and similarity between corresponding whole strands, only solvent exposed residues, only hydrophobic core residues, are shown as compared to the tenth domain of Fn3.

TABLE 1

| | | | | | | | | Only | | | |
|---|---|---|---|---|---|---|---|---|---|---|---|
| | | | | Only | | | | hydrophobic | | | |
| Whole | | | | solvent | | | | core | | | |
| strands | | | | exposed | | | | residues | | | |
| Strand A | Ident. | Sim. | Len. | Strand A | Ident. | Sim. | Len. | Strand A | Ident. | Sim. | Len. |
| fnIII_7 | 33 | 52 | 6 | fnIII_7 | 0 | 20 | 3 | fnIII_7 | 67 | 84 | 3 |
| fnIII_8 | 14 | 24 | 7 | fnIII_8 | 0 | 13 | 4 | fnIII_8 | 33 | 38 | 3 |
| fnIII_9 | 14 | 35 | 7 | fnIII_9 | 25 | 45 | 4 | fnIII_9 | 0 | 22 | 3 |
| Strand B | Ident. | Sim. | Len. | Strand B | Ident. | Sim. | Len. | Strand B | Ident. | Sim. | Len. |
| fnIII_7 | 43 | 61 | 7 | fnIII_7 | 25 | 38 | 4 | fnIII_7 | 67 | 91 | 3 |
| fnIII_8 | 14 | 42 | 7 | fnIII_8 | 0 | 8 | 4 | fnIII_8 | 33 | 87 | 3 |
| fnIII_9 | 29 | 46 | 7 | fnIII_9 | 25 | 17 | 4 | fnIII_9 | 33 | 84 | 3 |
| Strand C | Ident. | Sim. | Len. | Strand C | Ident. | Sim. | Len. | Strand C | Ident. | Sim. | Len. |
| fnIII_7 | 56 | 52 | 9 | fnIII_7 | 50 | 48 | 6 | fnIII_7 | 67 | 60 | 3 |
| fnIII_8 | 11 | 32 | 9 | fnIII_8 | 0 | 3 | 6 | fnIII_8 | 33 | 89 | 3 |
| fnIII_9 | 33 | 30 | 9 | fnIII_9 | 17 | 9 | 6 | fnIII_9 | 67 | 73 | 3 |
| Strand D | Ident. | Sim. | Len. | Strand D | Ident. | Sim. | Len. | Strand D | Ident. | Sim. | Len. |
| fnIII_7 | 33 | 26 | 6 | fnIII_7 | 25 | 25 | 4 | fnIII_7 | 50 | 27 | 2 |
| fnIII_8 | 33 | 64 | 6 | fnIII_8 | 50 | 58 | 4 | fnIII_8 | 0 | 77 | 2 |
| fnIII_9 | 33 | 27 | 6 | fnIII_9 | 25 | 32 | 4 | fnIII_9 | 50 | 17 | 2 |
| Strand E | Ident. | Sim. | Len. | Strand E | Ident. | Sim. | Len. | Strand E | Ident. | Sim. | Len. |
| fnIII_7 | 40 | 57 | 5 | fnIII_7 | 67 | 73 | 3 | fnIII_7 | 0 | 33 | 2 |
| fnIII_8 | 0 | 25 | 5 | fnIII_8 | 0 | 20 | 3 | fnIII_8 | 0 | 33 | 2 |
| fnIII_9 | 20 | 39 | 5 | fnIII_9 | 33 | 47 | 3 | fnIII_9 | 0 | 27 | 2 |
| Strand F | Ident. | Sim. | Len. | Strand F | Ident. | Sim. | Len. | Strand F | Ident. | Sim. | Len. |
| fnIII_7 | 44 | 67 | 9 | fnIII_7 | 40 | 60 | 5 | fnIII_7 | 50 | 75 | 4 |
| fnIII_8 | 33 | 53 | 9 | fnIII_8 | 20 | 35 | 5 | fnIII_8 | 50 | 75 | 4 |
| fnIII_9 | 22 | 55 | 9 | fnIII_9 | 0 | 29 | 5 | fnIII_9 | 50 | 87 | 4 |
| Strand G | Ident. | Sim. | Len. | Strand G | Ident. | Sim. | Len. | Strand G | Ident. | Sim. | Len. |
| fnIII_7 | 43 | 41 | 7 | fnIII_7 | 50 | 48 | 4 | fnIII_7 | 33 | 31 | 3 |
| fnIII_8 | 14 | 23 | 7 | fnIII_8 | 25 | 42 | 4 | fnIII_8 | 0 | −2 | 3 |
| fnIII_9 | 0 | 0 | 7 | fnIII_9 | 0 | 2 | 4 | fnIII_9 | 0 | −2 | 3 |

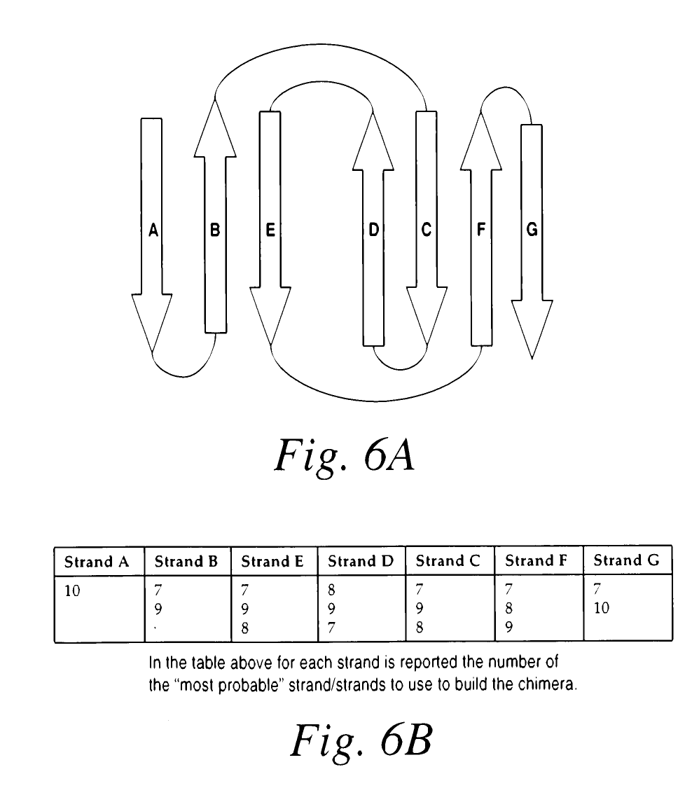

Based on the foregoing sequence identities/similarities, possible chimeras are shown in FIG. 6.

### EQUIVALENTS

Those skilled in the art will recognize or be able to ascertain, using no more than routine experimentation, many equivalents of the specific embodiments of the invention described herein. Such equivalents are intended to be encompassed by the following claims.

1. A conjugate comprising a fibronectin type III (Fn3)-based binding molecule linked to a non-Fn3 moiety, wherein the Fn3-based binding molecule comprises at least two Fn3 beta-strand domain sequences with a loop region sequence linked between each Fn3 beta-strand domain sequence, wherein the loop region sequence binds to a specific target.

2. The conjugate of claim 1, wherein the non-Fn3 moiety is capable of binding a second target.

3. The conjugate of claim 1, wherein the non-Fn3 moiety increases the half-life of the Fn3-based binding molecule when administered in vivo.

4. The conjugate of claim 1, wherein the non-Fn3 moiety comprises an antibody Fc region.

5. The conjugate of claim 4, wherein the antibody Fc region is fused to the Fn3-based binding molecule to a region selected from the group consisting of an N-terminal region and a C-terminal region.

6. The conjugate of claim 4, wherein the antibody Fc region is fused to the Fn3-based binding molecule at a region selected from the group consisting of a loop region, a beta-strand region, a beta-like strand, a C-terminal region, between the C-terminus and the most C-terminal beta strand or beta-like strand, an N-terminal region, and between the N-terminus and the most N-terminal beta strand or beta-like strand.

7. The conjugate of claim 4, wherein the half life of the conjugate is at least 5-fold, 10-fold, 15-fold, 20-fold, least 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold greater than that of the unconjugated Fn3-based binding molecule.

8. The conjugate of claim 4, wherein the half life of the conjugate is at least 5-30 fold greater than that of the unconjugated Fn3-based binding molecule.

9. The conjugate of claim 4, wherein the half life of the conjugate is at least 2-5 hours, 5-10 hours, 10-15 hours, 15-20 hours, 20-25 hours, 25-30 hours, 35-40 hours, 45-50 hours, 50-55 hours, 55-60 hours, 60-65 hours, 65-70 hours, 75-80 hours, 80-85 hours, 85-90 hours, 90-95 hours, 95-100 hours, 100-150 hours, 150-200 hours, 200-250 hours, 250-300 hours, 350-400 hours, 400-450 hours, 450-500 hours, 500-550 hours, 550-600 hours, 600-650 hours, 650-700 hours, 700-750 hours, 750-800 hours, 800-850 hours, 850-900 hours, 900-950 hours, 950-1000 hours, 1000-1050 hours, 1050-1100 hours, 1100-1150 hours, 1150-1200 hours, 1200-1250 hours, 1250-1300 hours, 1300-1350 hours, 1350-1400 hours, 1400-1450 hours, 1450-1500 hours greater than that of the unconjugated Fn3-based binding molecule.

10. The conjugate of claim 4, wherein the half life of the conjugate in vivo is at least 9.4 hours.

11. The conjugate of claim 1, wherein the non-Fn3 moiety comprises a Serum Albumin (SA), or transferrin, or portion thereof.

12. The conjugate of claim 11, wherein the Serum Albumin (SA), or portion thereof is Human Serum Albumin (HSA).

13. The conjugate of claim 12, wherein the HSA is conjugated to the Fn3-based binding molecule at a region selected from the group consisting of a loop region, a beta-strand region, a beta-like strand, a C-terminal region, between the C-terminus and the most C-terminal beta strand or beta-like strand, an N-terminal region, and between the N-terminus and the most N-terminal beta strand or beta-like strand.

14. The conjugate of claim 12, wherein the half life of the conjugate is at least 5-fold, 10-fold, 15-fold, 20-fold, least 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold greater than that of the unconjugated Fn3-based binding molecule.

15. The conjugate of claim 12, wherein the half life of the conjugate is at least 25-50 fold greater than that of the unconjugated Fn3-based binding molecule.

16. The conjugate of claim 12, wherein the half life of the conjugate is at least 2-5 hours, 5-10 hours, 10-15 hours, 15-20 hours, 20-25 hours, 25-30 hours, 35-40 hours, 45-50 hours, 50-55 hours, 55-60 hours, 60-65 hours, 65-70 hours, 75-80 hours, 80-85 hours, 85-90 hours, 90-95 hours, 95-100 hours, 100-150 hours, 150-200 hours, 200-250 hours, 250-300 hours, 350-400 hours, 400-450 hours, 450-500 hours, 500-550 hours, 550-600 hours, 600-650 hours, 650-700 hours, 700-750 hours, 750-800 hours, 800-850 hours, 850-900 hours, 900-950 hours, 950-1000 hours, 1000-1050 hours, 1050-1100 hours, 1100-1150 hours, 1150-1200 hours, 1200-1250 hours, 1250-1300 hours, 1300-1350 hours, 1350-1400 hours, 1400-1450 hours, 1450-1500 hours greater than that of the unconjugated Fn3-based binding molecule.

17. The conjugate of claim 12, wherein the half life of the conjugate in vivo is at least 19.6 hours.

18. The conjugate of claim 12, wherein polypeptide which binds Serum Albumin (SA), or transferrin, or portion thereof is an anti-Human Serum Albumin (HSA) polypeptide or an anti-transferrin polypeptide.

19. The conjugate of claim 18, wherein the anti-Human Serum Albumin (HSA) polypeptide or an anti-transferrin polypeptide is conjugated to the Fn3-based binding molecule at a region selected from the group consisting of a loop region, a beta-strand region, a beta-like strand, a C-terminal region, between the C-terminus and the most C-terminal beta strand or beta-like strand, an N-terminal region, and between the N-terminus and the most N-terminal beta strand or beta-like strand.

20. The conjugate of claim 18, wherein the half life of the conjugate is at least 5-fold, 10-fold, 15-fold, 20-fold, least 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold greater than that of the unconjugated Fn3-based binding molecule.

21. The conjugate of claim 18, wherein the half life of the conjugate is at least 10-35 fold greater than that of the unconjugated Fn3-based binding molecule.

22. The conjugate of claim 18, wherein the half life of the conjugate is at least 2-5 hours, 5-10 hours, 10-15 hours, 15-20 hours, 20-25 hours, 25-30 hours, 35-40 hours, 45-50 hours, 50-55 hours, 55-60 hours, 60-65 hours, 65-70 hours, 75-80 hours, 80-85 hours, 85-90 hours, 90-95 hours, 95-100 hours, 100-150 hours, 150-200 hours, 200-250 hours, 250-300 hours, 350-400 hours, 400-450 hours, 450-500 hours, 500-550 hours, 550-600 hours, 600-650 hours, 650-700 hours, 700-750 hours, 750-800 hours, 800-850 hours, 850-900 hours, 900-950 hours, 950-1000 hours, 1000-1050 hours, 1050-1100 hours, 1100-1150 hours, 1150-1200 hours, 1200-1250 hours, 1250-1300 hours, 1300-1350 hours, 1350-1400 hours, 1400-1450 hours, 1450-1500 hours greater than that of the unconjugated Fn3-based binding molecule.

23. The conjugate of claim 18, wherein the half life of the conjugate in vivo is at least 7.7 hours.

24. The conjugate of claim 1, wherein the non-Fn3 moiety comprises polyethylene glycol (PEG).

25. The conjugate of claim 24, wherein the PEG moiety is attached to a thiol group or an amine group.

26. The conjugate of claim 24, wherein the PEG moiety is attached to the Fn3-based binding molecule by site directed pegylation.

27. The conjugate of claim 24, wherein the PEG moiety is attached to a Cys residue.

28. The conjugate of claim 24, wherein the PEG moiety is attached to a non-natural amino acid residue.

29. The conjugate of claim 24, wherein a PEG moiety is attached on a region in the Fn3-based binding molecule selected from the group consisting of a loop region, a beta-strand region, a beta-like strand, a C-terminal region, between the C-terminus and the most C-terminal beta strand or beta-like strand, an N-terminal region, and between the N-terminus and the most N-terminal beta strand or beta-like strand.

30. The conjugate of claim 24, wherein the PEG moiety has a molecular weight of between about 2 kDa and about 100 kDa.

31. The conjugate of claim 24, wherein the half life of the conjugate is at least 5-fold, 10-fold, 15-fold, 20-fold, least 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold greater than that of the unconjugated Fn3-based binding molecule.

32. The conjugate of claim 24, wherein the half life of the conjugate is at least 5-25 fold greater than that of the unconjugated Fn3-based binding molecule.

33. The conjugate of claim 24, wherein the half life of the conjugate is at least 2-5 hours, 5-10 hours, 10-15 hours, 15-20 hours, 20-25 hours, 25-30 hours, 35-40 hours, 45-50 hours, 50-55 hours, 55-60 hours, 60-65 hours, 65-70 hours, 75-80 hours, 80-85 hours, 85-90 hours, 90-95 hours, 95-100 hours, 100-150 hours, 150-200 hours, 200-250 hours, 250-300 hours, 350-400 hours, 400-450 hours, 450-500 hours, 500-550 hours, 550-600 hours, 600-650 hours, 650-700 hours, 700-750 hours, 750-800 hours, 800-850 hours, 850-900 hours, 900-950 hours, 950-1000 hours, 1000-1050 hours, 1050-1100 hours, 1100-1150 hours, 1150-1200 hours, 1200-1250 hours, 1250-1300 hours, 1300-1350 hours, 1350-1400 hours, 1400-1450 hours, 1450-1500 hours greater than that of the unconjugated Fn3-based binding molecule.

34. The conjugate of claim 24, wherein the half life of the conjugate is at least 3.6 hours in vivo.

35. A conjugate with improved pharmacokinetic properties, the conjugate comprising: a fibronectin type III (Fn3)-based binding molecule linked to a polypeptide that binds to an antibody Fc region, wherein the Fn3-based binding molecule comprises at least two Fn3 beta-strand domain sequences with a loop region sequence linked between each Fn3 beta-strand domain sequence, wherein the conjugate binds to a specific target and has a serum half-life of at least 9.4 hours.

36. A conjugate with improved pharmacokinetic properties, the conjugate comprising: a fibronectin type III (Fn3)-based binding molecule linked to a Human Serum Albumin (HSA) moiety, wherein the Fn3-based binding molecule comprises at least two Fn3 beta-strand domain sequences with a loop region sequence linked between each Fn3 beta-strand domain sequence, wherein the conjugate binds to a specific target and has a serum half-life of at least 19.6 hours.

37. A conjugate with improved pharmacokinetic properties, the conjugate comprising: a fibronectin type III (Fn3)-based binding molecule linked to a polypeptide that binds to a Human Serum Albumin (HSA) moiety, wherein the Fn3-based binding molecule comprises at least two Fn3 beta-strand domain sequences with a loop region sequence linked between each Fn3 beta-strand domain sequence, wherein the conjugate binds to a specific target and has a serum half-life of at least 7.7 hours.

38. A conjugate with improved pharmacokinetic properties, the conjugate comprising: a fibronectin type III (Fn3)-based binding molecule linked to a PEG moiety, wherein the Fn3-based binding molecule comprises at least two Fn3 beta-strand domain sequences with a loop region sequence linked between each Fn3 beta-strand domain sequence, wherein the conjugate binds to a specific target and has a serum half-life of at least 3.6 hours.

39. A conjugate with improved pharmacokinetic properties, the conjugate comprising: a fibronectin type III (Fn3)-based binding molecule linked to an anti-FcRn moiety, wherein the Fn3-based binding molecule comprises at least two Fn3 beta-strand domain sequences with a loop region sequence linked between each Fn3 beta-strand domain sequence, and wherein the conjugate binds to neonatal FcR receptor (FcRn) with a high affinity at an acidic pH and with a low affinity at a neutral pH.

40. The conjugate of claim 39, wherein the acid pH ranges from about 1 to about 7.

41. The conjugate of claim 39, wherein the acid pH is about 6.

42. The conjugate of claim 39, wherein the neutral pH ranges from about 7 to about 8.

43. The conjugate of claim 39, wherein the neutral pH is about 7.4.

44. The Fn-3 based binding molecule or conjugate of any of the preceding claims, wherein the Fn3 domain is derived from at least two fibronectin modules.

45. The Fn-3 based binding molecule or conjugate of any of the preceding claims, wherein the Fn3 domain is derived from at least three or more fibronectin modules.

46. A nucleic acid comprising a sequence encoding a Fn-3 based binding molecule or conjugate of any of the preceding claims.

47. An expression vector comprising the nucleic acid of claim 46 operably linked with a promoter.

48. A cell comprising the nucleic acid of claim 47.

49. The cell according to claim 48, wherein the cell is a mammalian cell.

50. The cell according to claim 49, wherein the mammalian cell is a human mammalian cell.

51. The cell according to claim 49, wherein the mammalian cell is a CHO cell.

52. A method of producing a Fn-3 based binding molecule or conjugate of any of the preceding claims that binds to a target comprising: expressing a nucleic acid comprising a sequence encoding the Fn-3 based binding molecule or conjugate of any one of the preceding claims.

53. The method of claim 52 further comprising expressing the nucleic acid in a mammalian cell.

54. The method of claim 53, wherein the mammalian cell is a human mammalian cell.

55. The cell according to claim 53, wherein the mammalian cell is a CHO cell.

56. A composition comprising the Fn-3 based binding molecule or conjugate of any of the preceding claims, and a carrier.

57. A method of treating a subject for a disease selected from the group consisting of an autoimmune disease, an inflammation, a cancer, an infectious disease, a cardiovascular disease, a gastrointestinal disease, a respiratory disease, a metabolic disease, a musculoskeletal disease, a neurodegenerative disease, a psychiatric disease, an opthalmic disease and transplant rejection, the method comprising administering to the subject the binding molecule, conjugate, or composition of any preceding claims.

58. A method of detecting a protein in a sample comprising labeling the Fn-3 based binding molecule or conjugate of any of the preceding claims, contacting the labeled binding molecule or conjugate with the sample, and detecting complex formation between the binding molecule or conjugate with the protein.

59. (canceled)

60. (canceled)

61. (canceled)

62. (canceled)
